(19) United States
(12) Patent Application Publication
(10) Pub. No.: US 2022/0251191 A1
(43) Pub. Date: Aug. 11, 2022

(54) THERAPEUTIC COMPOSITIONS AND METHODS FOR TREATING CANCER IN COMBINATION WITH ANALOGS OF INTERLEUKIN PROTEINS

(71) Applicant: Arch Oncology, Inc., Brisbane, CA (US)

(72) Inventors: James BLINN, St. Louis, MO (US); John MCKEARN, St. Louis, MO (US); Joseph MONAHAN, St. Louis, MO (US); William R. STROHL, St. Louis, MO (US); Robyn PURO, St. Louis, MO (US); John RICHARDS, St. Louis, MO (US); Daniel S. PEREIRA, San Diego, CA (US); Juan C. ALMAGRO, Cambridge, MA (US)

(21) Appl. No.: 17/527,982

(22) Filed: Nov. 16, 2021

Related U.S. Application Data
(63) Continuation of application No. PCT/US2020/033317, filed on May 16, 2020.
(60) Provisional application No. 62/848,962, filed on May 16, 2019.
(60) Provisional application No. 62/848,975, filed on May 16, 2019.

(51) Int. Cl. C07K 16/28; C07K 14/54; A61K 45/06; A61P 35/00; A61K 39/395; A61K 38/20

(52) U.S. Cl. CPC C07K 16/2803; C07K 14/5418; C07K 14/5443; A61K 45/06; A61K 2039/505; A61K 39/3955; A61K 38/2046; A61K 38/2086; A61P 35/00

(57) ABSTRACT

Provided are compositions and methods for treating cancer by administering antiCD47 mAbs and anti-CD47 fusion proteins with distinct functional profiles or chimeric antigen receptor (CAR)-bearing immune effector cells in combination with analogs of interleukin proteins.

### CROSS REFERENCE TO RELATED APPLICATIONS

This application is a continuation of International Application No. PCT/US2020/033317, filed May 16, 2020, which claims priority to U.S. Provisional Application No. 62/848,962, filed May 16, 2019, and U.S. Provisional Application No. 62/848,975, filed May 16, 2019, each of which is incorporated by reference herein in its entirety for all purposes.

### FIELD

This disclosure is related generally to methods of treating cancer using anti-CD47 monoclonal antibodies (mAbs) and anti-CD47 fusion mAbs with distinct functional profiles or chimeric antigen receptor (CAR)-bearing immune effector cells in combination with analogs of interleukin proteins.

### SEQUENCE LISTING

The instant application contains a Sequence Listing which has been submitted electronically in ASCII format and is hereby incorporated by reference in its entirety. Said ASCII copy, created on Nov. 16, 2021, is named ARCO_013_02US_SeqList_ST25.txt and is 99.5 kilobytes in size.

### BACKGROUND

CD47 expression and/or activity have been implicated in a number of diseases and disorders. Many human cancers up-regulate cell surface expression of CD47 and those expressing the highest levels of CD47 appear to be the most aggressive and the most lethal for patients. Increased CD47 expression is thought to protect cancer cells from phagocytic clearance by sending a “don't eat me” signal to macrophages via SIRPα, an inhibitory receptor that prevents phagocytosis of CD47-bearing cells.

T cells can be genetically modified to express chimeric antigen receptors (CARs), which are fusion proteins comprised of an antigen recognition moiety and T cell activation domains. The CARs are designed to recognize antigens that are overexpressed on cancer cells.

CAR-Ts demonstrate exceptional clinical efficacy against B cell malignancies, and two therapies, KYMRIAH™ (tisagenlecleucel, Novartis) and YESCARTA™ (axicabtagene ciloleucel, Kite/Gilead), were recently approved by the FDA. Recent disclosures have also shown promise in expanding the use of CAR-T therapy to T-cell malignancies as well, and in enabling “off-the-shelf” use of pre-engineered cells from donors to treat malignancies without allogenic reactivity.

IL-7 is a cytokine important for B- and T-cell development. IL-7 is a hematopoietic growth factor produced by many cell types, including but not limited to being secreted by stromal cells in the bone marrow and thymus. IL-7 stimulates the differentiation of pluripotent hematopoietic stem cells into lymphoid progenitor cells. Binding of IL-7 with the IL-7 receptor results in a cascade of signals important for T-cell development within the thymus and survival in the periphery.

IL-15 induces proliferation and cytokine production in T and NK cells, as well as effector memory T-cell differentiation and sensitivity to apoptosis. IL-15Rα is widely expressed, for example by lymphoid cells, dendritic cells (DCs), fibroblasts, and epithelial, liver, intestine, and other cells and is thought to present IL-15 in trans to cells expressing IL-150 and γ chains.

IL-7, IL-15, and CD47 expression and/or activity have been implicated in a number of diseases and disorders. Accordingly, there exists a need for therapeutic compositions and methods for treating diseases and conditions associated with IL-7, IL-15, and CD47 in humans, including the prevention and treatment of various cancers. Disclosed herein are therapeutic uses for treating cancer in a subject in need thereof, comprising administering to the subject an anti-CD47 mAb and analogs of interleukin proteins, i.e., IL-7 and IL-15. Also disclosed herein are therapeutic uses for improving the expansion and persistence of immune effector cells, including chimeric antigen receptor (CAR)-bearing immune effector cells, as well as therapeutic uses for treating cancer in a subject in need thereof, comprising administering to the subject a population of immune effector cells, e.g., CAR-bearing immune effector cells and analogs of interleukin proteins, i.e., IL-7 and IL-15.

### SUMMARY

Embodiment 1. A polypeptide comprising: a first, and a second polypeptide chain, wherein the first polypeptide chain comprises (i) a first domain comprising a binding region of a light chain variable domain of an immunoglobulin (V_{L}) specific for human CD47; and (ii) a second light chain constant domain (C_{L}); and a second polypeptide chain comprising (i) a first domain comprising a binding region of a heavy chain variable region domain of an immunoglobulin (V_{H}) specific for human CD47, (ii) a second domain heavy chain constant domain (C_{H}); and (iii) a third domain comprising an IL-7 protein or variant thereof.

Embodiment 2. A polypeptide comprising: a first, and a second polypeptide chain, wherein the first polypeptide chain comprises (i) a first domain comprising a binding region of a light chain variable domain of an immunoglobulin (V_{L}) specific for human CD47, (ii) a second light chain constant domain (C_{L}); and (iii) a third domain comprising an IL-7 protein or variant thereof, and a second polypeptide chain comprising (i) a first domain comprising a binding region of a heavy chain variable region domain of an immunoglobulin (V_{H}) specific for human CD47; and (ii) a second domain heavy chain constant domain (C_{H}).

Embodiment 3. A polypeptide comprising: a first, and a second polypeptide chain, wherein the first polypeptide chain comprises (i) a first domain comprising an IL-7 protein or variant thereof, (ii) a second domain comprising a binding region of a light chain variable domain of an immunoglobulin (V_{L}) specific for human CD47; and (iii) a third domain light chain constant domain (C_{L}); and a second polypeptide chain comprising (i) a first domain comprising a binding region of a heavy chain variable region domain of an immunoglobulin (V_{H}) specific for human CD47; and (ii) a second domain heavy chain constant domain (C_{H}).

Embodiment 4. A polypeptide comprising: a first, and a second polypeptide chain, wherein the first polypeptide chain comprises (i) a first domain comprising a binding region of a light chain variable domain of an immunoglobulin (V_{L}) specific for human CD47; and (ii) a second light chain constant domain (C_{L}); and a second polypeptide chain comprising (i) a first domain comprising an IL-7 protein or variant thereof, (ii) a second domain comprising a binding region of a heavy chain variable region domain of an immunoglobulin (V_{H}) specific for human CD47; and (iii) a third domain heavy chain constant domain (C_{H}).

Embodiment 5. The peptide as recited in any of the Embodiments 1-4, wherein the IL-7 protein or variant thereof is modified.

Embodiment 6. The IL-7 protein or variant thereof as recited in any of the Embodiments 1 to 4, wherein the modified binding region for the IL-7 receptor is capable of binding to an IL-7 receptor to activate IL-7 signaling in a cell.

Embodiment 7. The IL-7 protein or variant thereof as recited in any of the Embodiments 1 to 4, wherein the modified binding region for the IL-7 receptor comprises a substitution in an amino acid.

Embodiment 8. The IL-7 protein or variant thereof as recited in any of the Embodiments 1 to 4, wherein the amino acid substitution in the modified binding region for the IL-7 receptor comprises a substitution in an amino acid position chosen from amino acid positions 10, 11, 14, 19, 81, and 85, wherein the amino acid position is relative to SEQ ID NO:2.

Embodiment 9. The IL-7 protein or variant thereof as recited in any of the Embodiments 1 to 4, wherein the amino acid substitution at amino acid position 10 is K10I, K10M, or K10V.

Embodiment 10. The IL-7 protein or variant thereof as recited in any of the Embodiments 1 to 4, wherein the amino acid substitution at amino acid position 10 is K10I.

Embodiment 11. The IL-7 protein or variant thereof as recited in any of the Embodiments 1 to 4, wherein the amino acid substitution at amino acid position 11 is Q11R.

Embodiment 12. The IL-7 protein or variant thereof as recited in any of the Embodiments 1 to 4, wherein the amino acid substitution at amino acid position 14 is S14T.

Embodiment 13. The IL-7 protein or variant thereof as recited in any of the Embodiments 1 to 4, wherein the amino acid substitution at amino acid position 19 is S19Q.

Embodiment 14. The IL-7 protein or variant thereof as recited in any of the Embodiments 1 to 4, wherein the amino acid substitution at amino acid position 81 is K81M or K81R.

Embodiment 15. The IL-7 protein or variant thereof as recited in any of the Embodiments 1 to 4, wherein the amino acid substitution at amino acid position 85 is G85M.

Embodiment 16. A method of treating cancer in a subject, comprising administering to the subject the polypeptide of any of Embodiments 1 to 15.

Embodiment 17. The method of Embodiment 16, wherein the cancer comprises a solid tumor.

Embodiment 18. The method of Embodiment 17, wherein the solid tumor is selected from the group consisting of cervical cancer, pancreatic cancer, ovarian cancer, mesothelioma, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma head and neck cancer, and any combination thereof.

Embodiment 19. The method of Embodiment 18, wherein the cancer is hematologic malignancy.

Embodiment 20. The method of Embodiment 19, wherein the hematologic malignancy is Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, or any combination thereof.

Embodiment 21. The method of Embodiment 19, wherein the hematologic malignancy is a T-cell malignancy.

Embodiment 22. The method of Embodiment 21, wherein the T-cell malignancy is T-cell acute lymphoblastic leukemia (T-ALL).

Embodiment 23. The method of Embodiment 21, wherein the T-cell malignancy is non-Hodgkins lymphoma.

Embodiment 24. The method of Embodiment 16, wherein the cancer is multiple myeloma.

Embodiment 25. The method of Embodiment 16, wherein the cancer is a B-cell malignancy.

Embodiment 26. The method of Embodiments 1 to 25, wherein the subject is further administered an anti-cancer agent.

Embodiment 27. The method of Embodiment 26, wherein the anti-cancer agent is a proteasome inhibitor.

Embodiment 28. The method of Embodiment 27, wherein the proteasome inhibitor is chosen from bortezomib, ixazomib, and carfilzomib.

Embodiment 29. The method of Embodiment 26, wherein the anti-cancer agent is an immune checkpoint inhibitor.

Embodiment 30. The method of Embodiment 29, wherein the immune checkpoint inhibitor is an inhibitor of PD-1, PD-L1, LAG-3, Tim-3, CTLA-4, or any combination thereof.

Embodiment 31. The method of Embodiment 29, wherein the immune checkpoint inhibitor is nivolumab, pembrolizumab, ipilimumab, atezolizumab, durvalumab, avelumab, tremelimumab, or any combination thereof.

Embodiment 32. The method of Embodiment 16, wherein the polypeptide is administered intravenously, intraperitoneally, intramuscularly, intraarterially, intrathecally, intralymphaticly, intralesionally, intracapsularly, intraorbitally, intracardiacly, intradermally, transtracheally, subcutaneously, subcuticularly, intraarticularly, subcapsularly, subarachnoidly, intraspinally, epidurally or intrasternally.

Embodiment 33. A pharmaceutical composition for use in treating a cancer in a subject in need thereof comprising a polypeptide of any one of Embodiments 1 to 4.

Embodiment 34. A for treating a cancer in a subject in need thereof comprising administering to the subject a therapeutically effective amount of:
a) an anti-CD47 antibody or antigen binding fragment thereof; and
b) an IL-7 protein.

Embodiment 35. The method of Embodiment 34, wherein the anti-CD47 antibody or antigen binding fragment thereof is comprising a combination of a heavy chain (HC) and a light chain (LC), wherein the combination is chosen from:
(i) a heavy chain comprising the amino acid sequence of SEQ ID NO:64 and a light chain comprising the amino acid sequence SEQ ID NO:68;
(ii) a heavy chain comprising the amino acid sequence of SEQ ID NO:65 and a light chain comprising the amino acid sequence SEQ ID NO:68;
(iii) a heavy chain comprising the amino acid sequence of SEQ ID NO:63 and a light chain comprising the amino acid sequence SEQ ID NO:67;
(iv) a heavy chain comprising the amino acid sequence of SEQ ID NO:64 and a light chain comprising the amino acid sequence SEQ ID NO:67;
(v) a heavy chain comprising the amino acid sequence of SEQ ID NO:65 and a light chain comprising the amino acid sequence SEQ ID NO:67; and
(vi) a heavy chain comprising the amino acid sequence of SEQ ID NO:66 and a light chain comprising the amino acid sequence SEQ ID NO:67.

Embodiment 36. The method of Embodiment 34, wherein the IL-7 protein has an amino acid sequence at least about 70%, at least about 80%, at least about 85%, at least about 90%, at least about 95%, at least about 96%, at least about 97%, at least about 98%, at least about 98%, at least about 99%, or about 100% identical to an amino acid sequence selected from SEQ ID NO. 1 (GenBank Accession No. P13232).

Embodiment 37. The method of Embodiments 34 or 35, wherein the IL-7 protein is modified.

Embodiment 38. The method of any one of Embodiments 34 to 36, wherein the IL-7 protein is a fusion protein.

Embodiment 39. The modified IL-7 protein of Embodiment 37, wherein the modified IL-7 protein is capable of binding to an IL-7 receptor to activate IL-7 signaling in a cell.

Embodiment 40. The modified interleukin protein of Embodiment 37, wherein the modified IL-7 protein comprises a substitution in an amino acid.

Embodiment 41. The modified interleukin protein of Embodiment 40, wherein the amino acid substitution in the modified IL-7 protein comprises a substitution in an amino acid position selected from the group consisting of amino acid positions 10, 11, 14, 19, 81, and 85, wherein the amino acid positions are relative to SEQ ID NO:2.

Embodiment 42. The modified interleukin protein of Embodiment 41, wherein the amino acid substitution at amino acid position 10 is K10I, K10M, or K10V.

Embodiment 43. The modified interleukin protein of Embodiment 42, wherein the amino acid substitution at amino acid position 10 is K10I.

Embodiment 44. The modified interleukin protein of Embodiment 41, wherein the amino acid substitution at amino acid position 11 is Q11R.

Embodiment 45. The modified interleukin protein of Embodiment 41, wherein the amino acid substitution at amino acid position 14 is S14T.

Embodiment 46. The modified interleukin protein of Embodiment 41, wherein the amino acid substitution at amino acid position 19 is S19Q.

Embodiment 47. The modified interleukin protein of Embodiment 41, wherein the amino acid substitution at amino acid position 81 is K81M or K81R.

Embodiment 48. The modified interleukin protein of Embodiment 41, wherein the amino acid substitution at amino acid position 85 is G85M.

Embodiment 49. The method of Embodiment 38, wherein the fusion protein comprises a heterologous moiety.

Embodiment 50. The method of Embodiment 49, wherein the heterologous moiety is a moiety extending a half-life of the IL-7 protein (“half-life extending moiety”).

Embodiment 51. The method of Embodiment 50, wherein the half-life extending moiety is selected from an Fc region of immunoglobulin or a part thereof, albumin, an albumin binding polypeptide, Pro/Ala/Ser (PAS), C-terminal peptide (CTP) of subunit of human chorionic gonadotropin, polyet9hylene glycol (PEG), long unstructured hydrophilic sequences of amino acids (XTEN), hydroxyethyl starch (HES), an albumin-binding small molecule, and a combination thereof.

Embodiment 52. The method of Embodiment 51, wherein the half-life extending moiety is an Fc domain.

Embodiment 53. The method of any one of Embodiments 36 to 52, wherein the IL-7 protein is to be administered at a weight-based dose between about 20 μg/kg and about 600 μg/kg or a flat dose of about 0.25 mg to about 9 mg.

Embodiment 54. The method of any one of Embodiments 36 to 53, wherein the IL-7 protein is to be administered at a weight-based dose of about 20 μg/kg, about 60 μg/kg, about 120 μg/kg, about 240 μg/kg, about 480 μg/kg, about 600 μg/kg, or about 10 mg/kg or a flat dose of about 0.25 mg, about 1 mg, about 3 mg, about 6 mg, or about 9 mg.

Embodiment 55. The method of any one of Embodiments 36 to 54, wherein the IL-7 protein is administered at a dosing interval of at least once a week, at least twice a week, at least three times a week, at least four times a week, at least once a month, or at least twice a month.

Embodiment 56. The method of any one of Embodiments 36 to 55, wherein the IL-7 protein is administered after an anti-CD47 antibody or antigen binding fragment thereof.

Embodiment 57. The method of any one of Embodiments 36 to 56, wherein the IL-7 protein is administered before an anti-CD47 antibody and antigen binding fragment thereof.

Embodiment 58. The method of any one of Embodiments 36 to 57, wherein the IL-7 protein is administered concurrently with an anti-CD47 antibody or antigen binding fragment thereof.

Embodiment 59. The method of Embodiment 34, wherein the cancer comprises a solid tumor.

Embodiment 60. The method of Embodiment 59, wherein the solid tumor is selected from the group consisting of cervical cancer, pancreatic cancer, ovarian cancer, mesothelioma, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma head and neck cancer, and any combination thereof.

Embodiment 61. The method of Embodiment 34, wherein the cancer is hematologic malignancy.

Embodiment 62. The method of Embodiment 61, wherein the hematologic malignancy is Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, or any combination thereof.

Embodiment 63. The method of Embodiment 61, wherein the hematologic malignancy is a T-cell malignancy.

Embodiment 64. The method of Embodiment 63, wherein the T-cell malignancy is T-cell acute lymphoblastic leukemia (T-ALL).

Embodiment 65. The method of Embodiment 63, wherein the T-cell malignancy is non-Hodgkins lymphoma.

Embodiment 66. The method of Embodiment 34, wherein the cancer is multiple myeloma.

Embodiment 67. The method of Embodiment 34, wherein the cancer is a B-cell malignancy.

Embodiment 68. The method of any one of Embodiments 34 to 67, wherein the subject is further administered an anti-cancer agent.

Embodiment 69. The method of Embodiment 68, wherein the anti-cancer agent is a proteasome inhibitor.

Embodiment 70. The method of Embodiment 69, wherein the proteasome inhibitor is chosen from bortezomib, ixazomib, and carfilzomib.

Embodiment 71. The method of Embodiment 68, wherein the anti-cancer agent is an immune checkpoint inhibitor.

Embodiment 72. The method of Embodiment 71, wherein the immune checkpoint inhibitor is an inhibitor of PD-1, PD-L1, LAG-3, Tim-3, CTLA-4, or any combination thereof.

Embodiment 73. The method of Embodiment 71, wherein the immune checkpoint inhibitor is nivolumab, pembrolizumab, ipilimumab, atezolizumab, durvalumab, avelumab, tremelimumab, or any combination thereof.

Embodiment 74. A modified IL-7 protein comprising at least one amino acid substitution comprising SEQ ID NOs:8-16.

Embodiment 75. The modified IL-7 protein of Embodiment 74, wherein the modified IL-7 protein is capable of binding to an IL-7 receptor to activate IL-7 signaling in a cell.

Embodiment 76. The modified IL-7 protein of any one of Embodiments 74 or 75, wherein the amino acid substitution in the modified IL-7 protein comprises a substitution in an amino acid position selected from the group consisting of amino acid positions 10, 11, 14, 19, 81, and 85, wherein the amino acid positions are relative to SEQ ID NO:2.

Embodiment 77. The modified IL-7 protein of Embodiment 76, wherein the amino acid substitution at amino acid position 10 is K10I, K10M, or K10V.

Embodiment 78. The modified IL-7 protein of Embodiment 77, wherein the amino acid substitution at amino acid position 10 is K10I.

Embodiment 79. The modified IL-7 protein of Embodiment 76, wherein the amino acid substitution at amino acid position 11 is Q11R.

Embodiment 80. The modified IL-7 protein of Embodiment 76, wherein the amino acid substitution at amino acid position 14 is S14T.

Embodiment 81. The modified IL-7 protein of Embodiment 76, wherein the amino acid substitution at amino acid position 19 is S19Q.

Embodiment 82. The modified IL-7 protein of Embodiment 76, wherein the amino acid substitution at amino acid position 81 is K81M or K81R.

Embodiment 83. The modified IL-7 protein of Embodiment 76, wherein the amino acid substitution at amino acid position 85 is G85M.

Embodiment 84. A nucleic acid construct encoding the protein of any one of Embodiments 77 to 83.

Embodiment 85. The nucleic acid construct of Embodiment 84, wherein the modified IL-7 protein further comprises a C-terminal Histidine tag.

Embodiment 86. The method of improving expansion and persistence of a chimeric antigen receptor (CAR)-bearing immune effector cell, comprising administering a CAR-bearing immune effector cell to a patient along with the protein of any one of Embodiments 77 to 83.

Embodiment 87. The method of initiating internal signaling in a CAR-bearing immune effector cell, comprising:
administering the protein of any one of Embodiments 77 to 83 to a patient in need thereof,
wherein the modified interleukin protein binds an IL-7 receptor; and
wherein binding of the modified interleukin protein initiates internal signaling in the cell.

Embodiment 88. The method of treating cancer in a subject in need thereof, comprising administering to the subject the protein of any one of Embodiments 77 to 83 and a CAR-bearing immune effector cell.

Embodiment 89. The method of any one of Embodiments 86 to 88, wherein the modified IL-7 protein is capable of binding to an IL-7 receptor to activate IL-7 signaling in a cell.

Embodiment 90. The method of any one of Embodiments 86 to 89, wherein the CAR-bearing immune effector cell is selected from a CAR-T cell, a CAR-iNKT cell, or a CAR-NK cell.

Embodiment 91. The method of any one of claims 86 to 90, wherein the modified interleukin protein and the CAR-bearing immune effector cell are administered concurrently with a drug.

Embodiment 92. A modified IL-15 protein comprising at least one amino acid substitution comprising SEQ ID NOs:31-45.

Embodiment 93. The modified interleukin protein of Embodiment 92, wherein the modified IL-15 protein is capable of binding to an IL-15 receptor to activate IL-15 signaling in a cell.

Embodiment 94. The modified interleukin protein of any one of Embodiments 92 or 93, wherein the amino acid substitution in the modified IL-15 protein comprises a substitution in an amino acid position selected from the group consisting of amino acid positions 3, 4, 11, 72, 79, and 112, wherein the amino acid positions are relative to SEQ ID NO:30.

Embodiment 95. The modified interleukin protein of Embodiment 94, wherein the amino acid substitution at amino acid position 3 is V3I, V3M, or V3R.

Embodiment 96. The modified interleukin protein of Embodiment 94, wherein the amino acid substitution at amino acid position 4 is N4H.

Embodiment 97. The modified interleukin protein of Embodiment 94, wherein the amino acid substitution at amino acid position 11 is K11L, K11M, or K11R.

Embodiment 98. The modified interleukin protein of Embodiment 94, wherein the amino acid substitution at amino acid position 72 is N72D, N72R or N72Y.

Embodiment 99. The modified interleukin protein of Embodiment 94, wherein the amino acid substitution at amino acid position 79 is N79E or N79S.

Embodiment 100. The modified interleukin protein of Embodiment 94, wherein the amino acid substitution at amino acid position 79 is N79S.

Embodiment 101. The modified interleukin protein of Embodiment 94, wherein the amino acid substitution at amino acid position 112 is N112H, N112M, or N112Y.

Embodiment 102. A nucleic acid construct encoding the modified interleukin protein of any one of Embodiments 95 to 101.

Embodiment 103. The nucleic acid construct of Embodiment 102, wherein the modified IL-15 protein further comprises an N-terminal Histidine tag.

Embodiment 104. The method of improving expansion and persistence of an immune effector cell, e.g., a chimeric antigen receptor (CAR)-bearing immune effector cell, comprising administering a CAR-bearing immune effector cell to a patient along with the modified interleukin protein of any one of Embodiments 95 to 101.

Embodiment 105. The method of initiating internal signaling in an immune effector cell, e.g., a CAR-bearing immune effector cell, comprising:
administering the modified interleukin protein of any one of Embodiments 95 to 101 to a patient in need thereof,
wherein the modified interleukin protein binds an IL-15 receptor; and
wherein binding of the modified interleukin protein initiates internal signaling in the cell.

Embodiment 106. The method of treating cancer in a subject in need thereof, comprising administering to the subject the modified interleukin protein of any one of Embodiments 95 to 101 and a CAR-bearing immune effector cell.

Embodiment 107. The method of any one of Embodiments 104 to 106, wherein the modified IL-15 protein is capable of binding to an IL-15 receptor to activate IL-15 signaling in a cell.

Embodiment 108. The method of any one of Embodiments 104 to 107, wherein the CAR-bearing immune effector cell effector cell is selected from a CAR-T cell, a CAR-iNKT cell, or a CAR-NK cell.

Embodiment 109. The method of any one of Embodiments 104 to 108, wherein the modified interleukin protein and the CAR-bearing immune effector cell are administered concurrently with a drug.

Embodiment 110. A polypeptide comprising:
an immunoglobulin variable region specific for human CD47 connected to at least one IL-7 protein, IL-7 variant, IL-15 protein, or IL-15 variant.

Embodiment 111. The polypeptide of Embodiment 110, wherein the immunoglobulin variable region specific for human CD47 is connected to an IL-7 protein, IL-7 variants, IL-15 protein, or IL-15 variant by a linker.

Embodiment 112. The polypeptide of Embodiment 123, wherein said linker is (GGGGS)n, wherein n=0, 1, 2, 3, 4, 5, 6, 7, 8, 9, 10, 11, 12, 13, 14, 15, 16, 17, 18.

Embodiment 113. The polypeptide of any one of Embodiments 110 to Embodiments 113, wherein said immunoglobulin variable region is specific is an anti-CD47 monoclonal antibody or fragment thereof.

### BRIEF DESCRIPTION OF THE DRAWINGS

The above and other aspects, features, and advantages of the present disclosure will be better understood from the following detailed descriptions taken in conjunction with the accompanying drawing(s), all of which are given by way of illustration only, and are not limitative of the present disclosure.

FIG. 1. Shows co-expression of IL-7Ra and IL-2Ry in Cos-7 Cells. Top row: IL-7Ra-positive cells (left), IL-2Rγ-positive cells (middle), and IL-7Rα/IL2Rγ double-positive cells (right) on day 1; Middle row: IL-7Rα-positive cells (left), IL-2Rγ-positive cells (middle), and IL-7Rα/IL2Rγ double-positive cells (right) on day 2; Bottom row: IL-7Rα-positive cells (left), IL-2Rγ-positive cells (middle), and IL-7Rα/IL-2Rγ double-positive cells (right) on day 3.

FIG. 2. Shows IL-7-induced pSTAT5 peripheral blood mononuclear cell (PBMC) signaling.

FIG. 3. Shows TR-FRET results of in-house prepared and commercial IL-7 binding to IL-7Rα. Top left: In-house prepared IL-7; Bottom left: Commercially obtained IL-7; Right: comparison of commercial vs in-house prepared IL-7.

FIG. 4. Shows binding of IL-7 mutant S1 to IL-7Rα compared to WT.

FIG. 5. Shows TR-FRET results of IL-7 binding to IL-7Ra with and without the γ subunit. Top left: IL-7 without γ; Bottom left: IL-7 with γ; Right: comparison of IL-7 with and without the γ subunit.

FIG. 6. Shows IL-15 pSTAT5 peripheral blood mononuclear cell (PBMC) signaling.

FIG. 7. Shows activity of WT IL-15 SEC purified fraction 6 in a pSTAT5 assay using human PBMCs.

FIG. 8. Shows example curves of confluence cytokine activity for IL-15 mutant M2 (N72R). Left side shows IL-15 M2 lot 11-20-18, N=1 in pg/mL; right side shows IL-15 M2 lot 11-20-18, N=2 in pg/mL.

FIG. 9. Shows example curves of confluence cytokine activity for IL-15 mutant M5 (N79S). Left side shows IL-15 M5 lot 11-20-18, N=1 in pg/mL; right side shows IL-15 M5 lot 11-20-18, N=2 in pg/mL.

FIG. 10. Shows TR-FRET results of in-house prepared and commercial IL-15 binding to IL-15Rβ. Top left: In-house prepared IL-15; Bottom left: Commercially obtained IL-15; Right: comparison of commercial vs in-house prepared IL-15.

FIG. 11. Shows binding of wild type IL-15 and IL-15 mutants M2 and M5 to IL-15Rβ. Top left: IL-15 mutant M2; Bottom left: WT IL-15; Right: IL-15 mutant M5.

FIG. 12. Shows TR-FRET results of in-house prepared IL-15 binding to IL-15Rβ with and without the γ subunit. Top left: IL-15 without γ; Bottom left: IL-15 with γ; Right: comparison of IL-15 with and without the γ subunit.

FIG. 13. Shows western blots of M-07e membrane extracts of the IL-15Rβ and γ receptors.

FIG. 14A. Shows a schematic of Anti-CD47-IL-7 Fusion Protein.

FIG. 14B. Shows a schematic of Anti-CD47-IL-7 Fusion Protein.

FIG. 14C. Shows a schematic of Anti-CD47-IL-7 Fusion Protein.

FIG. 14D. Shows a schematic of Anti-CD47-IL-7 Fusion Protein.

### DETAILED DESCRIPTION

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo or polynucleotide chemistry and hybridization described herein are those well-known and commonly used in the art.

Disclosed herein are modified interleukin-7 (IL-7) proteins.

In some embodiments, the modified IL-7 protein comprises an amino acid substitution set forth in Table 7 below.

In some embodiments, the amino acid substitution in the modified IL-7 protein comprises a substitution in an amino acid position selected from amino acid positions 10, 11, 14, 19, 81, and 85, wherein the amino acid positions are relative to SEQ ID NO:2.

In some embodiments, the amino acid substitution at amino acid position 10 is K10I, K10M, or K10V.

In some embodiments, the amino acid substitution at amino acid position 11 is Q11R.

In some embodiments, the amino acid substitution at amino acid position 14 is S 14T.

In some embodiments, the amino acid substitution at amino acid position 19 is S 19Q.

In some embodiments, the amino acid substitution at amino acid position 81 is K81M or K81R.

In some embodiments, the amino acid substitution at amino acid position 85 is G85M.

In some embodiments, the disclosure provides a nucleic acid construct encoding a modified IL-7 protein as described herein.

In some embodiments, the modified IL-7 protein further comprises a C-terminal histidine tag.

In some embodiments, the modified IL-7 protein is capable of binding to an IL-7 receptor to activate IL-7 signaling in a cell, wherein the IL-7 receptor is expressed on various cell types, including, but not limited to, naive and memory T cells.

In some embodiments, the binding of a modified IL-7 protein to an IL-7 receptor results in expansion or activation of the cell.

Disclosed herein are modified interleukin-15 (IL-15) proteins.

In some embodiments, the modified interleukin (IL) protein comprises an amino acid substitution set forth in Table 7 below.

In some embodiments, the amino acid substitution in the modified IL-15 subunit comprises a substitution in an amino acid position selected from the group consisting of amino acid positions 3, 4, 11, 72, 79, and 112, wherein the amino acid positions are relative to SEQ ID NO:30.

In some embodiments, the amino acid substitution at amino acid position 3 is V3I, V3M, or V3R.

In some embodiments, the amino acid substitution at amino acid position 4 is N4H.

In some embodiments, the amino acid substitution at amino acid position 11 is K11L, K11M, or K11R.

In some embodiments, the amino acid substitution at amino acid position 72 is N72D, N72R or N72Y.

In some embodiments, the amino acid substitution at amino acid position 79 is N79E or N79S.

In some embodiments, the amino acid substitution at amino acid position 112 is N112H, N112M, or N112Y.

In some embodiments, the disclosure provides a nucleic acid construct encoding a modified interleukin protein as described herein.

In some embodiments, the modified IL-15 protein further comprises an N-terminal histidine tag.

In some embodiments, the modified IL-15 protein is capable of binding to an IL-15 receptor to activate IL-15 signaling in a cell.

In some embodiments, the binding of a modified IL-15 protein to an IL-15 receptor results in expansion or activation of the cell, wherein the IL-7 receptor is expressed on dendritic cells, monocytes, and epithelial cells.

In some embodiments, the disclosure provides a method of improving expansion and persistence of a chimeric antigen receptor (CAR)-bearing immune effector cell comprising, administering a CAR-bearing immune effector cell to a patient in need thereof, with a modified IL-7 protein or modified IL-15 as described herein.

In some embodiments, the disclosure provides a method of initiating internal signaling in a CAR-bearing immune effector cell comprising, administering a modified IL-7 protein as described herein to a patient in need thereof, wherein the modified IL-7 protein binds an IL-7 receptor; and wherein binding of the modified IL-7 protein initiates internal signaling in the cell.

In some embodiments, the disclosure provides a method of initiating internal signaling in a CAR-bearing immune effector cell comprising, administering a modified IL-15 protein as described herein to a patient in need thereof, wherein the modified IL-15 protein binds an IL-15 receptor; and wherein binding of the modified IL-15 protein initiates internal signaling in the cell.

In some embodiments, the disclosure provides a method of treating cancer in a subject, comprising administering a CAR-bearing immune effector cell to a patient in need thereof, with a modified IL-7 protein or modified IL-15 protein as described herein.

In some embodiments, the CAR-bearing immune effector cell is selected from a CAR-T cell, a CAR-iNKT cell, or a CAR-NK cell.

In some embodiments, the modified IL-7 protein and the CAR-bearing immune effector cell are administered concurrently with a drug.

In some embodiments, the modified IL-7 protein and the CAR-bearing immune effector cell are administered with an antibody, i.e., an anti-CD47 antibody.

In some embodiments, the modified IL-15 protein and the CAR-bearing immune effector cell are administered concurrently with a drug.

In some embodiments, the modified IL-7 protein and the CAR-bearing immune effector cell are administered with an antibody, i.e., an anti-CD47 antibody.

Also disclosed herein are combination therapies, kits, and methods directed to a combination therapy comprising a population of immune effector cells, e.g., CAR-bearing immune effector cells, an interleukin protein, such as an IL-7 protein or an IL-15 protein, for the treatment of a disease.

In some embodiments, an IL-7 protein, IL-7 variant, or analogue thereof, may be administered in vivo to stimulate expansion of such immune effector cells, e.g., CAR-T cells or other CAR-bearing immune effector cells, in a patient receiving adoptive cell transfer therapy.

In some embodiments, an IL-15 protein, IL-15 variant, or analogue thereof, may be administered in vivo to stimulate expansion of such immune effector cells, e.g., CAR-T cells or other CAR-bearing immune effector cells, in a patient receiving adoptive cell transfer therapy.

In some embodiments, the disease can be a hyperproliferative disease or disorder, e.g., a cancer. The cancer may be a hematologic malignancy or solid tumor. The hematologic malignancy may be multiple myeloma and/or a T-cell malignancy. The T-cell malignancy may be T-cell acute lymphoblastic leukemia (T-ALL) and/or non-Hodgkin's lymphoma. The solid tumor or hematological malignancy. The solid tumor may be cervical cancer, pancreatic cancer, ovarian cancer, mesothelioma, or lung cancer.

In some embodiments, the present disclosure includes a kit comprising a population of chimeric antigen receptor (CAR)-bearing immune effector cells for use in combination with an IL-7 protein, IL-7 variant, or analogue thereof, wherein the kit further comprises instructions according to any methods disclosed herein.

### IL-7 and IL-7 Analogs

Disclosed herein are modified/mutant interleukin-7 (IL-7) proteins. Such modified IL-7 proteins may also be referred to herein as mutant IL-7 proteins (e.g., IL-7 variants, IL-7 functional fragment, IL-7 derivatives, or any combination thereof, e.g., fusion protein, chimeric protein, etc.) as long as the IL-7 protein contains one or more biological activities of IL-7, e.g., capable of binding to IL-7R, e.g., inducing early T-cell development, promoting T-cell homeostasis. See El Kassar and Gress. J Immunotoxicol. 2010 March; 7(1): 1-7.

Disclosed herein are combinations and uses in combination of anti-CD47 antibodies with native and/or modified IL-7 protein.

Also disclosed herein are combinations and uses in combination of immune effector cells, e.g., CAR-bearing immune effector cells, such as CAR-T cells, CAR-iNKT cells, or CAR-NK cells, with native and/or modified IL-7 protein. Such combinations stimulate of expansion of CAR-T cells and other CAR-bearing immune effector cells in a patient receiving adoptive cell transfer therapy.

IL-7 binds to its receptor, which is composed of the two chains IL-7Ra (CD127), shared with the thymic stromal lymphopoietin (TSLP) (Ziegler and Liu, 2006), and the common γ chain (γc, CD132) for IL-2, IL-15, IL-9, and IL-21. Whereas γc is expressed by most hematopoietic cells, IL-7Rα is nearly exclusively expressed on lymphoid cells. After binding to its receptor, IL-7 signals through two different pathways: Jak-Stat (Janus kinase-Signal transducer and activator of transcription) and PI3K/Akt responsible for differentiation and survival, respectively. The absence of IL-7 signaling is responsible for a reduced thymic cellularity as observed in mice that have received an anti-IL-7 neutralizing monoclonal antibody (MAb); Grabstein et al., 1993), in IL-7−/−(von Freeden-Jeffry et al., 1995), IL-7Rα−/−(Peschon et al., 1994; Maki et al., 1996), γc−/−(Malissen et al., 1997), and Jak3−/− mice (Park et al., 1995). In the absence of IL-7 signaling, mice lack T-, B-, and NK-T cells. IL-7α−/− mice (Peschon et al., 1994) have a similar but more severe phenotype than IL-7−/− mice (von Freeden-Jeffry et al., 1995), possibly because TSLP signaling is also abrogated in IL-7α−/− mice. IL-7 is required for the development of γδ cells (Maki et al., 1996) and NK-T cells (Boesteanu et al., 1997).

In some embodiments, the IL-7 protein includes a polypeptide comprising the amino acid sequence as set forth in any one of SEQ ID NOs:8-16. In some embodiments, the IL-7 protein comprises an amino acid sequence having a sequence identity of about 70%, about 75%, about 80%, about 85%, about 90%, about 91%, about 92%, about 93%, about 94%, about 95%, about 96%, about 97%, about 98%, or about 99% or higher, to a sequence of SEQ ID NOs:8-16.

In some embodiments, the IL-7 protein includes a modified IL-7 or a fragment thereof, wherein the modified IL-7 or the fragment retains one or more biological activities of wild-type IL-7. In some embodiments, the IL-7 protein can be derived from humans, rats, mice, monkeys, cows, or sheep.

In some embodiments, the human IL-7 may have an amino acid sequence represented by SEQ ID NO:1 (Genbank Accession No. P13232).

In some embodiments, the present disclosure provides modified interleukin (IL) proteins comprising an amino acid substitution. A “modified interleukin”, such as a “modified IL-7,” may also be referred to herein as a mutant interleukin, such as a mutant IL-7, and the modified or mutant interleukin protein may have at least one amino acid substitution. The modified or mutant interleukin protein retains the activity of the native, unmodified interleukin protein, such that the modified interleukin activates signaling pathways in a cell.

As described herein, an amino acid substitution useful in accordance with the disclosure may be any amino acid substitution. In some embodiments, a modified IL-7 may have a single amino acid substitution, or may have 2, 3, 4, or more amino acid substitutions. In some embodiments, a particular amino acid may be substituted for another amino acid with similar properties, i.e., a polar amino acid being substitute for another polar amino acid, or a nonpolar amino acid being substituted for another nonpolar amino acid. In some embodiments, an amino acid substitution useful for the present disclosure may be a substitution set forth in Table 7.

In some embodiments, an interleukin protein that may be modified or into which a mutation may be introduced may be, for example, IL-7. In some embodiments, the modified IL-7 protein may be capable of binding to the native receptor of the IL protein. For example, a modified IL-7 protein as described herein would bind to the native IL-7 receptor to activate IL-7 signaling in a cell.

In some embodiments, an amino acid substitution in a modified IL-7 as described herein may comprise a substitution in an amino acid position selected from the group consisting of amino acid positions 10, 11, 14, 19, 81, and 85, wherein the amino acid positions are relative to SEQ ID NO:2. For example, in some embodiments, an amino acid substitution at amino acid position 10 may be K10I, K10M, or K10V. In some embodiments, an amino acid substitution at amino acid position 11 may be Q11R. In some embodiments, an amino acid substitution at amino acid position 14 may be S14T. In some embodiments, an amino acid substitution at amino acid position 19 may be S19Q. In some embodiments, an amino acid substitution at amino acid position 81 may be K81M or K81R. In some embodiments, an amino acid substitution at amino acid position 85 may be G85M.

In some embodiments, a modified IL-7 as described herein retains activity or function similar or substantially similar to a native IL-7. For example, in some embodiments, a modified IL-7 as described herein is capable of initiating internal signaling in a cell. In some embodiments, internal signaling in the cell results in expansion or activation of the cell.

In accordance with the invention, a nucleic acid is also provided that encodes a modified IL-7 as described herein. Such a nucleic acid may be able to be introduced into, reproduced by, and expressed in any appropriate host, such as a bacterial (i.e., E. coli cells) or eukaryotic host cell (i.e., mammalian cells). A nucleic acid that encodes a modified IL-7 may be provided in a vector for production in a host cell. Such a vector may have any elements necessary as appropriate for expression and replication in the host cell. Such elements, as well as methods for their use, are well known and available in the art.

In some embodiments, a modified IL-7 protein may be further modified by the addition of a protein tag. Protein tagging and associated methods are well known in the art. In some embodiments, a modified IL-7 may be engineered to contain a histidine tag at either the C terminal or the N-terminal, or both. For example, a modified IL-7 protein as described herein may be modified to have a C-terminal histidine tag.

In some embodiments, an IL-7 or variant thereof may comprise a mutation in a specific amino acid position. In some embodiments, a mutant IL-7 may have an amino acid substitution in one or more of amino acid positions 10, 11, 14, 19, 81, and 85. In some embodiments, a mutant IL-7 may have an amino acid substitution in more than one of amino acid positions 10, 11, 14, 19, 81, and 85. For example, in some embodiments, an IL-7 variant or mutant may have an amino acid substitution as set forth in Table 7.

In some embodiments, the IL-7 may have a structure that includes a polypeptide having a biological activity of IL-7 and an oligopeptide consisting of 1 to 10 amino acids. In some embodiments, the IL-7 may have an amino acid sequence chosen from SEQ ID NOs:8-16. Additionally, the IL-7 protein may comprise an amino acid sequence having a homology of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, and at least about 99%, to the amino acid sequences of SEQ ID NOs:8-16.

In some embodiments, the IL-7 protein is encoded by a nucleic acid molecule encoding the IL-7 protein. The nucleic acid molecule may be one encoding the polypeptide having an amino acid sequence chosen from SEQ ID NOs:8-16, or one with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% homology to those sequences. The nucleic acid molecule may include a polynucleotide sequence chosen from SEQ ID NOs:8-16, or one with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% homology to those sequences. The nucleic acid molecule may further include a signal sequence or a leader sequence.

In some embodiments, the central hydrophobic region includes 4 to 12 hydrophobic residues, which immobilize the signal sequence through a membrane lipid bilayer during the translocation of an immature polypeptide. After the initiation, the signal sequence can be frequently cut off within the lumen of ER by a cellular enzyme known as a signal peptidase. In particular, the signal sequence may be a secretory signal sequence for tissue plasminogen activation (tPa), signal sequence of herpes simplex virus glycoprotein D (HSV gDs), or a growth hormone. Preferably, the secretory signal sequence used in higher eukaryotic cells including mammals, etc., may be used. Additionally, in some embodiments, as the secretory signal sequence, the signal sequence included in the wild type IL-7 may be used or it may be used after substituting with a codon with high expression frequency in a host cell.

The IL-7 protein useful for the present disclosure, in some embodiments, can be encoded by an expression vector comprising a nucleic acid molecule encoding the IL-7 protein. The expression vector may be RcCMV (Invitrogen, Carlsbad) or a variant thereof. The expression vector may include a human cytomegalovirus (CMV) for promoting continuous transcription of a target gene in a mammalian cell and a polyadenylation signal sequence of bovine growth hormone for increasing the stability state of RNA after transcription. In some embodiments, the expression vector is pAD15, which is a modified form of RcCMV.

The IL-7 protein useful for the present disclosure, in some embodiments, can be expressed by a host cell including the expression vector. An appropriate host cell can be used for the expression and/or secretion of a target protein, by the transduction or transfection of the DNA sequence.

Examples of the appropriate host cell to be used, in some embodiments, may include immortal hybridoma cell, NS/0 myeloma cell, 293 cell, Chinese hamster ovary (CHO)cell, HeLa cell, human amniotic fluid-derived cell (Cap T cell) or COS cell.

The IL-7 protein useful for the disclosure, in some embodiments, can be made by culturing the transformed cells by the expression vector; and harvesting the IL-7 protein from the culture or the cells obtained from the culturing process.

The IL-7 protein useful for the disclosure, in some embodiments, may be purified from the culture medium or cell extract. For example, after obtaining the supernatant of the culture medium, in which a recombinant protein was secreted, the supernatant may be concentrated a protein concentration filter available in the commercial market, e.g., an Amicon or Millipore Pellicon ultrafiltration unit. Then, the concentrate may be purified by a method known in the art. For example, the purification may be performed using a matrix coupled to protein A.

The IL-7 protein useful for the disclosure, in some embodiments, can be prepared by including a linker of an amino acid sequence having 1 to 10 amino acid residues consisting of methionine, glycine, serine or a combination thereof, to the N-terminal of a polypeptide having the activity of IL-7 or a similar activity thereof.

When the linker is a peptide linker, in some embodiments, the connection may occur in any linking region. They may be coupled using a crosslinking agent known in the art. In some embodiments, examples of the crosslinking agent may include N-hydroxy succinimide esters such as 1,1-bis(diazoacetyl)-2-phenylethane, glutaraldehyde, and 4-azidosalicylic acid; imido esters including disuccinimidyl esters such as 3,3′-dithiobis (succinimidyl propionate), and bifunctional maleimides such as bis-Nmaleimido-1,8-octane, but is not limited thereto.

Additionally, in some embodiments, the linker may be an albumin linker.

When the linker is formed by a chemical bond, the chemical bond maybe a disulfide bond, a diamine bond, a sulfide-amine bond, a carboxy-amine bond, an ester bond, and a covalent bond.

The above preparation method may further include a step of linking a polynucleotide encoding a polypeptide consisting of a heterogeneous sequence with an IL-7 protein. In particular, the polypeptide consisting of a heterogeneous sequence may be anyone selected from an Fc region of immunoglobulin or a part thereof, albumin, an albumin binding polypeptide, PAS, a CTP of the β subunit of human chorionic gonadotropin, PEG, XTEN, HES, an albumin binding small molecule, and a combination thereof.

The IL-7 protein may be administered in combination with an anti-CD47 antibody or antigen binding fragment thereof.

The IL-7 protein may be administered for promoting the expansion or survival of immune effector cells, e.g., chimeric antigen receptor (CAR)-bearing immune effector cells, in particular, engineered CAR-T cells, CAR-iNKT cells, or CAR-NK cells.

The IL-7 protein useful for the disclosure, in some embodiments, further include a pharmaceutically acceptable carrier. The pharmaceutically acceptable carrier may be any non-toxic material which is suitable for the delivery into patients. The carrier may be distilled water, alcohols, fats, waxes, or inactive solids. Additionally, any pharmaceutically acceptable adjuvants (buffering agents, dispersing agents) may also be contained therein.

Additionally, the pharmaceutical composition containing the IL-7 protein may be administered to subjects by various methods. Also, the pharmaceutical composition containing the IL-7 protein and an anti-CD47 monoclonal antibody and antigen binding fragment thereof may be administered to subjects by various methods. For example, the composition may be parenterally administered, e.g., subcutaneously, intramuscularly, or intravenously, e.g., intramuscularly. The composition may be sterilized by a conventional sterile method. The composition may contain a pharmaceutically acceptable auxiliary material and an adjuvant required for the regulation of physiological conditions such as pH adjustment, a toxicity-adjusting agent, and an analog thereof. Specific examples may include sodium acetate, potassium chloride, calcium chloride, sodium lactate, etc. The concentration of the IL-7 protein to be included in the formulations may vary widely. For example, the concentration of the IL-7 protein may be less than about 0.5%, and generally or at least about 1% to as much as 15% to 20%, depending on the weight. The concentration may be selected based on the selected particular administration methods, fluid volumes, viscosities, etc.

The present method includes administering a therapeutically effective amount of the IL-7 protein in combination with an anti-CD47 antibody, or antigen-binding fragment thereof, to a subject in need thereof, who has a health state related or unrelated to the target disease. The subject may be a mammal, and preferably a human.

Compositions may be administered by appropriate routes. Compositions may be provided by a direct administration (e.g., locally by an administration via injection, transplantation, or local administration into a tissue region) or system (e.g., parenterally or orally) via an appropriate means. In some embodiments, the IL-7 protein can be administered intravenously, subcutaneously, intraocularly, intraperitoneally, intramuscularly, orally, intrarectally, intraorbitally, intracerebrally, intracranially, intraspinally, intraventricularly, intrathecally, intracistenally, intracapsularly, intranasally, or aerosol administration. In other embodiments, the composition is formulated to contain an aqueous or physiologically applicable suspension of body fluids or a part of the solution thereof. As such, the physiologically acceptable carrier or transporter can be added into the composition and delivered to patients, and this does not cause a negative effect on the electrolyte and/or volume balance of patients. Accordingly, the physiologically acceptable carrier or transporter may be a physiological saline. Anti-CD47 antibodies, or antigen-binding fragments thereof, will be administered by injection or infusion, typically intravenously.

For reconstituting or complementing the functions of a desired protein, an expression vector capable of expressing a fusion protein in a particular cell may be administered along with any biologically effective carrier. This may be any formulation or composition that can efficiently deliver a gene encoding a desired protein or an IL-7 fusion protein into a cell in vivo.

The unit dose of the modified IL-7 or an IL-7 fusion protein may be in the range of 0.001 mg/kg to 10 mg/kg. In one embodiment, a therapeutically effective amount of the IL-7 protein to be used in combination therapy with an anti-CD47 antibody, or antigen-binding fragment thereof, can be in the range of 0.01 mg/kg to 2 mg/kg. In another embodiment, the therapeutically effective amount of the protein, for humans, maybe in the range of 0.02 mg/kg to 1 mg/kg, e.g., 20 μg/kg to 600 μg/kg, e.g., 60 μg/kg to 600 μg/kg. In some embodiments, a therapeutically effective amount of an IL-7 protein is about 10 mg/kg. In other embodiments, a therapeutically effective amount of an IL-7 protein is about 20 μg/kg, about 60 μg/kg, about 120 μg/kg, about 240 μg/kg, about 480 μg/kg, or about 600 μg/kg. In other embodiments, a therapeutically effective amount of an IL-7 protein is about a flat dose of about 0.25 mg, about 1 mg, about 3 mg, about 6 mg, or about 9 mg. In other embodiments, a therapeutically effective amount of an IL-7 protein is a flat dose. In some embodiments, a therapeutically effective amount of an IL-7 protein is about 0.25 mg to about 9 mg, e.g., about 0.25 mg, about 1 mg, about 3 mg, about 6 mg, or about 9 mg. In some embodiments, the therapeutically effective amount may vary depending on the subject diseases for treatment and the presence of adverse effects. In some embodiments, the administration of the IL-7 protein may be performed by periodic bolus injections or external reservoirs (e.g., intravenous bags) or by continuous intravenous, subcutaneous, or intraperitoneal administration from the internal (e.g., bio corrosive implants).

In certain embodiments, the IL-7 protein is administered at a dosing interval of at least a week, at least two weeks, at least three weeks, at least four weeks, at least five weeks, at least six weeks, at least seven weeks, at least eight weeks, at least nine weeks, or at least ten weeks.

In other embodiments, the IL-7 protein can be administered repeatedly. In other embodiments, the IL-7 protein is administered at least two times, at least three times, at least four times, at least five times, at least six times, at least seven times, at least eight times, at least nine times, or at least ten times.

In certain embodiments, the IL-7 protein can be formulated: for example, about 3 mg/ml to about 100 mg/ml an IL-7 protein, about 20 mM sodium citrate, about 5 w/v % sucrose, about 1 to 2w/v % sorbitol or mannitol, and about 0.05w/v % Tween 80 or poloxamer at a pH of about 5.0.

In some embodiments, the IL-7 protein and anti-CD47 antibody, or antigen-binding fragment thereof, can be administered in combination with other drug(s) or physiologically active material(s) which have a preventative or treating effect on the disease to be prevented or treated, or may be formulated into a combined preparation in combination with other drug(s), for example, may be administered in combination with an immunostimulant such as a hematopoietic growth factor, a cytokine, an antigens, and an adjuvant. The hematopoietic growth factor may be a stem cell factor (SCF), a G-CSF, a GM-CSF, or an Flt-3 ligand. The cytokine may be γ interferon, IL-2, IL-15, IL-21, IL-12, RANTES, or B7-1.

### IL-15 and IL-15 Analogs

Disclosed herein are modified/mutant interleukin-15 (IL-15) proteins. Such modified IL-15 proteins may also be referred to herein as mutant IL-15 proteins (e.g., IL-15 variants, IL-15 mutants, IL-15 functional fragments, IL-15 derivatives, or any combination thereof, e.g., fusion protein, chimeric protein, etc.) as long as the IL-15 protein contains one or more biological activities of IL-15, e.g., capable of binding to IL-15R, e.g., inducing early T-cell development, promoting T-cell homeostasis.

Disclosed herein are combinations and uses in combination of anti-CD47 antibodies with native and/or modified IL-7 protein.

Also disclosed herein are combinations and uses in combination of immune effector cells, e.g., CAR-bearing immune effector cells, such as CAR-T cells, CAR-iNKT cells, or CAR-NK cells, with native and/or modified IL-15 protein. Such combinations stimulate of expansion of CAR-T cells and other CAR-bearing immune effector cells in a patient receiving adoptive cell transfer therapy.

In some embodiments, the present disclosure provides modified interleukin (IL) proteins comprising an amino acid substitution. A “modified interleukin”, such as a “modified IL-15,” may also be referred to herein as a mutant interleukin, such as a mutant IL-15, and the modified or mutant interleukin protein may have at least one amino acid substitution. The modified or mutant interleukin protein retains the activity of the native, unmodified interleukin protein, such that the modified interleukin activates signaling pathways in a cell.

As described herein, an amino acid substitution useful in accordance with the disclosure may be any amino acid substitution. In some embodiments, a modified IL-15 may have a single amino acid substitution, or may have 2, 3, 4, or more amino acid substitutions. In some embodiments, a particular amino acid may be substituted for another amino acid with similar properties, i.e., a polar amino acid being substitute for another polar amino acid, or a nonpolar amino acid being substituted for another nonpolar amino acid. In some embodiments, an amino acid substitution useful for the present disclosure may be a substitution as set forth in Table 7.

IL-15 binds to its receptor, which is composed of the IL-15Ra chain, the IL-15Rβ chain (CD122), and the common γ chain, which is shared with IL-7R. The IL-15Rα has a particularly high affinity for IL-15 in comparison with all other cytokine receptors, due to an unusual structure of the IL-15Rα chain having an additional region called the sushi domain. The action on the sushi-domain receptor (IL-15Rα) is transduced into the cell via the Jak1 and Jak3 kinases that phosphorylate STAT-3, STAT-5, and STAT-6 nuclear factors and, with the help of some mitogen-activated kinases (MAPK), result in enhanced transcription of IL-15-dependent genes in the cell nucleus.

In some embodiments, the modified IL-15 protein may be capable of binding to the native receptor of the IL protein. For example, modified IL-15 protein as described herein would bind to the native IL-15 receptor to activate IL-15 signaling in a cell.

IL-15 induces proliferation and cytokine production in T and NK cells, as well as effector memory T-cell differentiation and sensitivity to apoptosis. IL-15Rα is widely expressed, for example by lymphoid cells, dendritic cells (DCs), fibroblasts, and epithelial, liver, intestine, and other cells and is thought to present IL-15 in trans to cells expressing IL-150 and γ chains.

In some embodiments, a modified IL-15 as described herein retains activity or function similar or substantially similar to a native IL-15. For example, in some embodiments, a modified IL-15 as described herein is capable of initiating internal signaling in a cell. In some embodiments, internal signaling in the cell results in expansion or activation of the cell.

In some embodiments, the IL-15 protein includes a polypeptide comprising the amino acid sequence as set forth in any one of SEQ ID NOs:31-45. In some embodiments, the IL-15 protein comprises an amino acid sequence having a sequence identity of about 70%, about 75%, about 80%, about 85%, about 90%, about 91%, about 92%, about 93%, about 94%, about 95%, about 96%, about 97%, about 98%, or about 99% or higher, to a sequence of SEQ ID NOs:17-31.

In some embodiments, the IL-15 protein includes a modified IL-15 or a fragment thereof, wherein the modified IL-15 or the fragment retains one or more biological activities of wild-type IL-15. In some embodiments, the IL-15 protein can be derived from humans, rats, mice, monkeys, cows, or sheep.

In some embodiments, the human IL-15 may have an amino acid sequence represented by SEQ ID NO:29 (Genbank Accession No. P40933).

In accordance with the invention, a nucleic acid is also provided that encodes a modified IL-15 as described herein. Such a nucleic acid may be able to be introduced into, reproduced by, and expressed in any appropriate host, such as a bacterial (i.e., E. coli cells) or eukaryotic host cell (i.e., mammalian cells). A nucleic acid that encodes a modified IL-15 may be provided in a vector for production in a host cell. Such a vector may have any elements necessary as appropriate for expression and replication in the host cell. Such elements, as well as methods for their use, are well known and available in the art.

In some embodiments, a modified IL-15 protein may be further modified by the addition of a protein tag. Protein tagging and associated methods are well known in the art. In some embodiments, a modified IL-15 may be engineered to contain a histidine tag at either the C terminal or the N-terminal, or both. In some embodiments, a modified IL-15 protein as described herein may be modified to have an N-terminal Histidine tag.

In some embodiments, an IL-15 or variant thereof may comprise a mutation in a specific amino acid position. In some embodiments, a mutant IL-15 may have an amino acid substitution in one or more of amino acid positions 3, 4, 11, 72, 79, and 112. In some embodiments, a mutant IL-15 may have an amino acid substitution in more than one of amino acid positions 3, 4, 11, 72, 79, and 112. For example, in some embodiments, an IL-15 variant or mutant may have an amino acid substitution as set forth in Table 7.

In some embodiments, an amino acid substitution in a modified IL-15 as described herein may comprise a substitution in an amino acid position selected from the group consisting of amino acid positions 3, 4, 11, 72, 79, and 112, wherein the amino acid positions are relative to SEQ ID NO:30 In some embodiments, an amino acid substitution at amino acid position 3 may be V3I, V3M, or V3R. In some embodiments, an amino acid substitution at amino acid position 4 may be N4H. In some embodiments, an amino acid substitution at amino acid position 11 may be K11L, K11M, or K11R. In some embodiments, an amino acid substitution at amino acid position 72 may be N72D, N72R or N72Y. In some embodiments, an amino acid substitution at amino acid position 79 may be N79E or N79S. In some embodiments, an amino acid substitution at amino acid position 112 is N112H, N112M, or N112Y. One of skill in the art would be able to identify amino acid substitutions that may be beneficial in accordance with the present disclosure.

In some embodiments, the IL-15 may have a structure that includes a polypeptide having a biological activity of IL-15 and an oligopeptide consisting of 1 to 10 amino acids. In some embodiments, the IL-15 may have an amino acid sequence chosen from SEQ ID NOs:31-45. Additionally, the IL-15 protein may comprise an amino acid sequence having a homology of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, and at least about 99%, to the amino acid sequences of SEQ ID NOs:31-45.

In some embodiments, the IL-15 protein is encoded by a nucleic acid molecule encoding the IL-15 protein. The nucleic acid molecule may be one encoding the polypeptide having an amino acid sequence chosen from SEQ ID NOs:31-45, or one with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% homology to those sequences. The nucleic acid molecule may include a polynucleotide sequence chosen from SEQ ID NOs:31-45, or one with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% homology to those sequences. The nucleic acid molecule may further include a signal sequence or a leader sequence.

In some embodiments, the central hydrophobic region includes 4 to 12 hydrophobic residues, which immobilize the signal sequence through a membrane lipid bilayer during the translocation of an immature polypeptide. After the initiation, the signal sequence can be frequently cut off within the lumen of ER by a cellular enzyme known as a signal peptidase. In particular, the signal sequence may be a secretory signal sequence for tissue plasminogen activation (tPa), signal sequence of herpes simplex virus glycoprotein D (HSV gDs), or a growth hormone. Preferably, the secretory signal sequence used in higher eukaryotic cells including mammals, etc., may be used. Additionally, in some embodiments, as the secretory signal sequence, the signal sequence included in the wild type IL-15 may be used or it may be used after substituting with a codon with high expression frequency in a host cell.

The IL-15 protein useful for the present disclosure, in some embodiments, can be encoded by an expression vector comprising a nucleic acid molecule encoding the IL-15 protein. The expression vector may be RcCMV (Invitrogen, Carlsbad) or a variant thereof. The expression vector may include a human cytomegalovirus (CMV) for promoting continuous transcription of a target gene in a mammalian cell and a polyadenylation signal sequence of bovine growth hormone for increasing the stability state of RNA after transcription. In some embodiments, the expression vector is pAD15, which is a modified form of RcCMV.

The IL-15 protein useful for the present disclosure, in some embodiments, can be expressed by a host cell including the expression vector. An appropriate host cell can be used for the expression and/or secretion of a target protein, by the transduction or transfection of the DNA sequence.

Examples of the appropriate host cell to be used, in some embodiments, may include immortal hybridoma cell, NS/0 myeloma cell, 293 cell, Chinese hamster ovary (CHO)cell, HeLa cell, human amniotic fluid-derived cell (Cap T cell) or COS cell.

The IL-15 protein useful for the disclosure, in some embodiments, can be made by culturing the transformed cells by the expression vector; and harvesting the IL-15 protein from the culture or the cells obtained from the culturing process.

The IL-15 protein useful for the disclosure, in some embodiments, may be purified from the culture medium or cell extract. For example, after obtaining the supernatant of the culture medium, in which a recombinant protein was secreted, the supernatant may be concentrated a protein concentration filter available in the commercial market, e.g., an Amicon or Millipore Pellicon ultrafiltration unit. Then, the concentrate may be purified by a method known in the art. For example, the purification may be performed using a matrix coupled to protein A.

The IL-15 protein useful for the disclosure, in some embodiments, can be prepared by including a linking oligopeptide of an amino acid sequence having 1 to 10 amino acid residues consisting of methionine, glycine, or a combination thereof, to the N-terminal of a polypeptide having the activity of IL-15 or a similar activity thereof.

When the linker is a peptide linker, in some embodiments, the connection may occur in any linking region. They may be coupled using a crosslinking agent known in the art. In some embodiments, examples of the crosslinking agent may include N-hydroxy succinimide esters such as 1,1-bis(diazoacetyl)-2-phenylethane, glutaraldehyde, and 4-azidosalicylic acid; imido esters including disuccinimidyl esters such as 3,3′-dithiobis (succinimidyl propionate), and bifunctional maleimides such as bis-Nmaleimido-1,8-octane, but is not limited thereto.

Additionally, in some embodiments, the linker may be an albumin linker.

When the linker is formed by a chemical bond, the chemical bond maybe a disulfide bond, a diamine bond, a sulfide-amine bond, a carboxy-amine bond, an ester bond, and a covalent bond.

The above preparation method may further include a step of linking a polynucleotide encoding a polypeptide consisting of a heterogeneous sequence with an IL-15 protein. In particular, the polypeptide consisting of a heterogeneous sequence may be anyone selected from the group consisting of an Fc region of immunoglobulin or a part thereof, albumin, an albumin binding polypeptide, PAS, a CTP of the β subunit of human chorionic gonadotropin, PEG, XTEN, HES, an albumin binding small molecule, and a combination thereof.

The IL-15 protein may be administered in combination with an anti-CD47 antibody or antigen binding fragment thereof.

The IL-15 protein may be administered for promoting the expansion or survival of immune effector cells, e.g., chimeric antigen receptor (CAR)-bearing immune effector cells, in particular, engineered CAR-T cells, CAR-iNKT cells, or CAR-NK cells.

The IL-15 protein useful for the disclosure, in some embodiments, further include a pharmaceutically acceptable carrier. The pharmaceutically acceptable carrier may be any non-toxic material which is suitable for the delivery into patients. The carrier may be distilled water, alcohols, fats, waxes, or inactive solids. Additionally, any pharmaceutically acceptable adjuvants (buffering agents, dispersing agents) may also be contained therein.

Additionally, the pharmaceutical composition containing the IL-15 protein may be administered to subjects by various methods. Also, the pharmaceutical composition containing the IL-15 protein and an anti-CD47 monoclonal antibody and antigen binding fragment thereof may be administered to subjects by various methods. For example, the composition may be parenterally administered, e.g., subcutaneously, intramuscularly, or intravenously, e.g., intramuscularly. The composition may be sterilized by a conventional sterile method. The composition may contain a pharmaceutically acceptable auxiliary material and an adjuvant required for the regulation of physiological conditions such as pH adjustment, a toxicity-adjusting agent, and an analog thereof. Specific examples may include sodium acetate, potassium chloride, calcium chloride, sodium lactate, etc. The concentration of the IL-15 protein to be included in the formulations may vary widely. For example, the concentration of the IL-15 protein may be less than about 0.5%, and generally or at least about 1% to as much as 15% to 20%, depending on the weight. The concentration may be selected based on the selected particular administration methods, fluid volumes, viscosities, etc.

The present method includes administering a therapeutically effective amount of the IL-15 protein in combination with an anti-CD47 antibody, or antigen-binding fragment thereof, to a subject in need thereof, who has a health state related or unrelated to the target disease. The subject may be a mammal, and preferably a human.

Compositions may be administered by appropriate routes. Compositions may be provided by a direct administration (e.g., locally by an administration via injection, transplantation, or local administration into a tissue region) or system (e.g., parenterally or orally) via an appropriate means. In some embodiments, the IL-15 protein can be administered intravenously, subcutaneously, intraocularly, intraperitoneally, intramuscularly, orally, intrarectally, intraorbitally, intracerebrally, intracranially, intraspinally, intraventricularly, intrathecally, intracistenally, intracapsularly, intranasally, or aerosol administration. In other embodiments, the composition is formulated to contain an aqueous or physiologically applicable suspension of body fluids or a part of the solution thereof. As such, the physiologically acceptable carrier or transporter can be added into the composition and delivered to patients, and this does not cause a negative effect on the electrolyte and/or volume balance of patients. Accordingly, the physiologically acceptable carrier or transporter may be a physiological saline. Anti-CD47 antibodies, or antigen-binding fragments thereof, will be administered by injection or infusion, typically intravenously.

For reconstituting or complementing the functions of a desired protein, an expression vector capable of expressing a fusion protein in a particular cell may be administered along with any biologically effective carrier. This may be any formulation or composition that can efficiently deliver a gene encoding a desired protein or an IL-15 fusion protein into a cell in vivo.

The unit dose of the modified IL-15 or an IL-15 fusion protein may be in the range of 0.001 mg/kg to 10 mg/kg. In one embodiment, a therapeutically effective amount of the IL-15 protein to be used in combination therapy with an anti-CD47 antibody, or antigen-binding fragment thereof, can be in the range of 0.01 mg/kg to 2 mg/kg. In another embodiment, the therapeutically effective amount of the protein, for humans, maybe in the range of 0.02 mg/kg to 1 mg/kg, e.g., 20 μg/kg to 600 μg/kg, e.g., 60 μg/kg to 600 μg/kg. In some embodiments, a therapeutically effective amount of an IL-15 protein is about 10 mg/kg. In other embodiments, a therapeutically effective amount of an IL-15 protein is about 20 μg/kg, about 60 μg/kg, about 120 μg/kg, about 240 μg/kg, about 480 μg/kg, or about 600 μg/kg. In other embodiments, a therapeutically effective amount of an IL-15 protein is about a flat dose of about 0.25 mg, about 1 mg, about 3 mg, about 6 mg, or about 9 mg. In other embodiments, a therapeutically effective amount of an IL-15 protein is a flat dose. In some embodiments, a therapeutically effective amount of an IL-15 protein is about 0.25 mg to about 9 mg, e.g., about 0.25 mg, about 1 mg, about 3 mg, about 6 mg, or about 9 mg. In some embodiments, the therapeutically effective amount may vary depending on the subject diseases for treatment and the presence of adverse effects. In some embodiments, the administration of the IL-15 protein may be performed by periodic bolus injections or external reservoirs (e.g., intravenous bags) or by continuous intravenous, subcutaneous, or intraperitoneal administration from the internal (e.g., biocorrosive implants).

In certain embodiments, the IL-15 protein is administered at a dosing interval of at least a week, at least two weeks, at least three weeks, at least four weeks, at least five weeks, at least six weeks, at least seven weeks, at least eight weeks, at least nine weeks, or at least ten weeks.

In other embodiments, the IL-15 protein can be administered repeatedly. In other embodiments, the IL-15 protein is administered at least two times, at least three times, at least four times, at least five times, at least six times, at least seven times, at least eight times, at least nine times, or at least ten times.

In certain embodiments, the IL-15 protein can be formulated: for example, about 3 mg/ml to about 100 mg/ml an IL-15 protein, about 20 mM sodium citrate, about 5 w/v % sucrose, about 1 to 2w/v % sorbitol or mannitol, and about 0.05w/v % Tween 80 or poloxamer at a pH of about 5.0.

In some embodiments, the IL-15 protein and anti-CD47 antibody, or antigen-binding fragment thereof, can be administered in combination with other drug(s) or physiologically active material(s) which have a preventative or treating effect on the disease to be prevented or treated, or may be formulated into a combined preparation in combination with other drug(s), for example, may be administered in combination with an immunostimulant such as a hematopoietic growth factor, a cytokine, an antigens, and an adjuvant. The hematopoietic growth factor may be a stem cell factor (SCF), a G-CSF, a GM-CSF, or an Flt-3 ligand. The cytokine may be γ interferon, IL-2, IL-15, IL-21, IL-12, RANTES, or B7-1.

### CD47 Antibodies

Many human cancers up-regulate cell surface expression of CD47 and those expressing the highest levels of CD47 appear to be the most aggressive and the most lethal for patients. Increased CD47 expression is thought to protect cancer cells from phagocytic clearance by sending a “don't eat me” signal to macrophages via SIRPα, an inhibitory receptor that prevents phagocytosis of CD47-bearing cells (Oldenborg et al. Science 288: 2051-2054, 2000; Jaiswal et al. (2009) Cell 138(2):271-851; Chao et al. (2010) Science Translational Medicine 2(63):63ra94). Thus, the increase of CD47 expression by many cancers provides them with a cloak of “selfness” that slows their phagocytic clearance by macrophages and dendritic cells.

Antibodies that block CD47 and prevent its binding to SIRPα have shown efficacy in human tumor in murine (xenograft) tumor models. Such blocking anti-CD47 mAbs exhibiting this property increase the phagocytosis of cancer cells by macrophages, which can reduce tumor burden (Majeti et al. (2009) Cell 138 (2): 286-99; U.S. Pat. No. 9,045,541; Willingham et al. (2012) Proc Natl Acad. Sci. USA 109(17):6662-6667; Xiao et al. (2015) Cancer Letters 360:302-309; Chao et al. (2012) Cell 142:699-713; Kim et al. (2012) Leukemia 26:2538-2545) and may ultimately lead to generation of an adaptive immune response to the tumor (Tseng et al. (2013) Proc Natl Acad. Sci. USA 110 (27):11103-11108; Soto-Pantoja et al. (2014) Cancer Res. 74 (23): 6771-6783; Liu et al. (2015) Nat. Med. 21 (10): 1209-1215).

However, there are mechanisms by which anti-CD47 mAbs can attack transformed cells that have not yet been exploited in the treatment of cancer. Multiple groups have shown that particular anti-human CD47 mAbs induce cell death of human tumor cells. Anti-CD47 mAb Ad22 induces cell death of multiple human tumor cells lines (Pettersen et al. J Immuno. 166: 4931-4942, 2001; Lamy et al. J. Biol. Chem. 278: 23915-23921, 2003). AD22 was shown to indice rapid mitochondrial dysfunction and rapid cell death with early phosphatidylserine exposure and a drop in mitochondrial membrane potential (Lamy et al. J Biol. Chem. 278: 23915-23921, 2003). Anti-CD47 mAb MABL-2 and fragments thereof induce cell death of human leukemia cell lines, but not normal cells in vitro and had an anti-tumor effect in in vivo xenograft models. (Uno et al. (2007) Oncol. Rep. 17 (5): 1189-94). Anti-human CD47 mAb 1F7 induces cell death of human T-cell leukemias (Manna and Frazier (2003) J. Immunol. 170: 3544-53) and several breast cancers (Manna and Frazier (2004) Cancer Research 64 (3):1026-36). 1F7 kills CD47-bearing tumor cells without the action of complement or cell mediated killing by NK cells, T-cells, or macrophages. Instead, anti-CD47 mAb 1F7 acts via a non-apoptotic mechanism that involves a direct CD47-dependent attack on mitochondria, discharging their membrane potential and destroying the ATP-generating capacity of the cell leading to rapid cell death. It is noteworthy that anti-CD47 mAb 1F7 also blocks binding of SIRPα to CD47 (Rebres et al et al. J. Cellular Physiol. 205: 182-193, 2005) and thus it can act via two mechanisms: (1) direct tumor toxicity, and (2) causing phagocytosis of cancer cells. A single mAb that can accomplish both functions may be superior to one that only blocks CD47/SIRPα binding.

The present disclosure includes anti-CD47 mAbs know in the art and anti-CD47 mAbs with distinct functional profiles, as described in U.S. Pat. No. 10,239,945 and US Patent Publication US20180142019, in combination with modified/mutant IL-7 or modified/mutant IL-15 proteins. These anti-CD47 mAbs described herein possess one or more distinct combinations of properties chosen from: 1) exhibit cross-reactivity with one or more species homologs of CD47; 2) block the interaction between CD47 and its ligand SIRPα; 3) increase phagocytosis of human tumor cells, 4) induce death of susceptible human tumor cells; 5) do not induce cell death of human tumor cells; 6) have reduced binding to human red blood cells (hRBCs); 7) have no detectable binding to hRBCs; 8) cause reduced agglutination of hRBCs; 9) cause no detectable agglutination of hRBCs; 10) reverse TSP1 inhibition of the nitric oxide (NO) pathway and/or 11) do not reverse TSP1 inhibition of the NO pathway.

In another embodiment, the anti-CD47 antibodies or antigen binding fragments thereof, are those comprising a combination of a heavy chain (HC) and a light chain (LC), wherein the combination is chosen from:

(i) a heavy chain comprising the amino acid sequence of SEQ ID NO:64 and a light chain comprising the amino acid sequence SEQ ID NO:68;

(ii) a heavy chain comprising the amino acid sequence of SEQ ID NO:65 and a light chain comprising the amino acid sequence SEQ ID NO:68;

(iii) a heavy chain comprising the amino acid sequence of SEQ ID NO:63 and a light chain comprising the amino acid sequence SEQ ID NO:67;

(iv) a heavy chain comprising the amino acid sequence of SEQ ID NO:64 and a light chain comprising the amino acid sequence SEQ ID NO:67;

(v) a heavy chain comprising the amino acid sequence of SEQ ID NO:65 and a light chain comprising the amino acid sequence SEQ ID NO:67; and

(vi) a heavy chain comprising the amino acid sequence of SEQ ID NO:66 and a light chain comprising the amino acid sequence SEQ ID NO:67.

>Vx9humH12 Full length HC
(SEQ ID NO: 63)
QVQLVQSGAEVKKPGASVKVSCKASGYTFTNYWIHWVRQAPGQGLEWMGY
TDPRTDYTEYNQKFKDRVTMTRDTSTSTVYMELSSLRSEDTAVYYCARGG
RVGLGYWGQGTLVTVSSASTKGPSVFPLAPCSRSTSESTAALGCLVKDYF
PEPVTVSWNSGALTSGVHTFPAVLQSSGLYSLSSVVTVPSSNFGTQTYTC
NVDHKPSNTKVDKTVERKCCVECPPCPAPPVAGPSVFLFPPKPKDTLMIS
RTPEVTCVVVDVSHEDPEVQFNWYVDGVEVHNAKTKPREEQFNSTFRVVS
VLTVVHQDWLNGKEYKCKVSNKGLPAPIEKTISKTKGQPREPQVYTLPPS
REEMTKNQVSLTCLVKGFYPSDIAVEWESNGQPENNYKTTPPMLDSDGSF
FLYSKLTVDKSRWQQGNVFSCSVMHEALHNHYTQKSLSLSPGK.
>Vx9humH14 Full Length Heavy Chain
(SEQ ID NO: 64)
EVQLVQSGAEVKKPGESLKISCKGSGYTFTNYWIHWVRQMPGKGLEWMGY
TDPRTDYTEYNQKFKDQVTISADKSISTAYLQWSSLKASDTAMYYCARGG
RVGLGYWGQGTLVTVSSASTKGPSVFPLAPCSRSTSESTAALGCLVKDYF
PEPVTVSWNSGALTSGVHTFPAVLQSSGLYSLSSVVTVPSSNFGTQTYTC
NVDHKPSNTKVDKTVERKCCVECPPCPAPPVAGPSVFLFPPKPKDTLMIS
RTPEVTCVVVDVSHEDPEVQFNWYVDGVEVHNAKTKPREEQFNSTFRVVS
VLTVVHQDWLNGKEYKCKVSNKGLPAPIEKTISKTKGQPREPQVYTLPPS
REEMTKNQVSLTCLVKGFYPSDIAVEWESNGQPENNYKTTPPMLDSDGSF
FLYSKLTVDKSRWQQGNVFSCSVMHEALHNHYTQKSLSLSPGK.
>Vx9humH15 Full Length Heavy Chain
(SEQ ID NO: 65)
QVQLVQSGAEVKKPGSSVKVSCKASGYTFTNYWIHWVRQAPGQGLEWMGY
TDPRTDYTEYAQKFQGRVTITADESTSTAYMELSSLRSEDTAVYYCARGG
RVGLGYWGQGTLVTVSSASTKGPSVFPLAPCSRSTSESTAALGCLVKDYF
PEPVTVSWNSGALTSGVHTFPAVLQSSGLYSLSSVVTVPSSNFGTQTYTC
NVDHKPSNTKVDKTVERKCCVECPPCPAPPVAGPSVFLFPPKPKDTLMIS
RTPEVTCVVVDVSHEDPEVQFNWYVDGVEVHNAKTKPREEQFNSTFRVVS
VLTVVHQDWLNGKEYKCKVSNKGLPAPIEKTISKTKGQPREPQVYTLPPS
REEMTKNQVSLTCLVKGFYPSDIAVEWESNGQPENNYKTTPPMLDSDGSF
FLYSKLTVDKSRWQQGNVFSCSVMHEALHNHYTQKSLSLSPGK.
>Vx9humH16 Full Length Heavy Chain
(SEQ ID NO: 66)
EVQLVQSGAEVKKPGESLKISCKGSGYTFTNYWIHWVRQMPGKGLEWMGY
TDPRTDYTEYSPSFQGQVTISADKSISTAYLQWSSLKASDTAMYYCARGG
RVGLGYWGQGTLVTVSSASTKGPSVFPLAPCSRSTSESTAALGCLVKDYF
PEPVTVSWNSGALTSGVHTFPAVLQSSGLYSLSSVVTVPSSNFGTQTYTC
NVDHKPSNTKVDKTVERKCCVECPPCPAPPVAGPSVFLFPPKPKDTLMIS
RTPEVTCVVVDVSHEDPEVQFNWYVDGVEVHNAKTKPREEQFNSTFRVVS
VLTVVHQDWLNGKEYKCKVSNKGLPAPIEKTISKTKGQPREPQVYTLPPS
REEMTKNQVSLTCLVKGFYPSDIAVEWESNGQPENNYKTTPPMLDSDGSF
FLYSKLTVDKSRWQQGNWSCSVMHEALHNHYTQKSLSLSPGK.
>Vx9humL02 Full Length Light Chain
(SEQ ID NO: 67)
DIVMTQSPDSLAVSLGERATINCRSSQNIVQSNGNTYLEWYQQKPGQPPK
LLIYKVFHRFSGVPDRFSGSGSGTDFTLTISSLQAEDVAVYYCFQGSHVP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.
>Vx9humL07 Full Length Light Chain
(SEQ ID NO: 68)
DVVMTQSPLSLPVTLGQPASISCRSSQNIVQSNGNTYLEWFQQRPGQSPR
RLIYKVFHRFSGVPDRFSGSGSGTDFTLKISRVEAEDVGVYYCFQGSHVP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKFIKVYAC
EVTHQGLSSPVTKSFNRGEC.

### Anti-CD47-IL-7 Fusion Proteins

The anti-CD47-IL-7 fusion proteins disclosed herein comprise a human heavy chain variable domain and a light chain variable domain combined with a human kappa or any human Fc Ig constant domain, respectively. A glycine-serine linker (G4S)n at either the C or N terminus of the light or heavy antibody chain was designed to link IL-7 (wildtype or mutant) to the antibody polypeptide. These fusion constructs were designed to incorporate a secretion signal and cloned into a mammalian expression system, and transfected into CHO cells to generate antibody fusion proteins. The protein variants were expressed, secreted into the medium, and purified using protein A resin.

Models of humanized mice can be utilized for determining efficacy of anti-CD47-IL-7 fusion proteins. This humanized mouse model expresses human CD47 and human SIRP so the SIRP/CD47 axis will be blocked by an anti-CD47 antibody. This humanized mouse model is a syngeneic model, wherein there is an intact immune system which allows for the assessment of IL-7 contribution to tumor efficacy.

In a double-humanized mouse model, B-hSIRPα/hCD47, human extracellular domains of SIRPα and CD47 replace their respective murine counterparts. Homozygous B-hSIRPα/hCD47 mice express humanized but not the wild type mouse SIRPα and CD47. Use of these mice allows for assessment of human specific CD47 mAbs along with assessment of the role of adaptive immune responses. An example of a tumor model could be MC38-hCD47 cell line that expresses human CD47 in MC38 colon carcinoma cells. During testing, an anti-human CD47 and anti-human SIRPα antibodies were efficacious in controlling MC38-hCD47 tumor growth in B-hSIRPα/hCD47 mice.

In another example, a mouse can be transplanted with a human immune system. This would allow the implantation of xenograft tumors and assess AO-176 and IL-7 contribution to anti-tumor efficacy.

NSG mice are humanized by adoptive transfer using human umbilical cord blood-derived CD34+ stem cells from a qualified source, following myeloablation treatment. CD34+ stem cells develop into human immune cells that engraft within the immunodeficient NSG mice. Models engrafted with cord blood-derived hematopoietic stem cells (HSC) develop multi-lineage engraftment and display robust T-cell maturation and T-cell dependent inflammatory responses.

Lastly, a syngeneic model can be developed where testing can utilize a murine-specific cd47-IL7 fusion.

### Chimeric Antigen Receptor (CAR)-Bearing Immune Effector Cells

Disclosed herein are modified/mutant IL-7 or modified/mutant IL-15 proteins with and uses in combination with CAR-bearing immune effector cells, such as a CAR-T cells, a CAR-iNKT cells, or a CAR-NK cells. The following sections describe examples of CAR-bearing immune effector cells to be used with modified/mutant IL-7 and IL-15 proteins.

A CAR-T cell is a T cell that expresses a chimeric antigen receptor. The phrase “chimeric antigen receptor (CAR),” as used herein, refers to a recombinant fusion protein that has an antigen-specific extracellular domain coupled to an intracellular domain that directs the cell to perform a specialized function upon binding of an antigen to the extracellular domain. The terms “artificial T cell receptor,” “chimeric T-cell receptor,” and “chimeric immunoreceptor” may each be used interchangeably herein with the term “chimeric antigen receptor.” Chimeric antigen receptors are distinguished from other antigen binding agents by their ability to both bind MHC-independent antigen and transduce activation signals via their intracellular domain. The extracellular and intracellular portions of a CAR are discussed in more detail below.

The antigen-specific extracellular domain of a chimeric antigen receptor recognizes and specifically binds an antigen, typically a surface-expressed antigen of a malignancy. An antigen-specific extracellular domain specifically binds an antigen when, for example, it binds the antigen with an affinity constant or affinity of interaction (KD) between about 0.1 pM to about 10 M, preferably about 0.1 pM to about 1 M, more preferably about 0.1 pM to about 100 nM. Methods for determining the affinity of interaction are known in the art. An antigen-specific extracellular domain suitable for use in a CAR of the present disclosure may be any antigen-binding polypeptide, a wide variety of which are known in the art. An antigen-specific extracellular domain suitable for use in a CAR of the present disclosure may be any antigen-binding polypeptide, a wide variety of which are known in the art. In some instances, the antigen-binding domain is a single chain Fv (scFv). Other antibody-based recognition domains (cAb VHH (camelid antibody variable domains) and humanized versions thereof, 1gNAR VH (shark antibody variable domains) and humanized versions thereof, sdAb VH (single domain antibody variable domains) and “camelized” antibody variable domains are suitable for use. In some instances, T-cell receptor (TCR) based recognition domains such as single chain TCR (scTv, single chain two-domain TCR containing V.alpha.V.beta.) are also suitable for use.

Suitable antigens may include T cell-specific antigens and/or antigens that are not specific to T cells. In one preferred embodiment, an antigen specifically bound by the chimeric antigen receptor of a CAR-T cell, and the antigen for which the CAR-T cell is deficient, is an antigen expressed on a malignant T cell, more preferably an antigen that is overexpressed on malignant T cell in comparison to a non-malignant T cell. A “malignant T cell” is a T cell derived from a T-cell malignancy. The term “T-cell malignancy” refers to a broad, highly heterogeneous grouping of malignancies derived from T-cell precursors, mature T cells, or natural killer cells. Non-limiting examples of T-cell malignancies include T-cell acute lymphoblastic leukemia/lymphoma (T-ALL), T-cell large granular lymphocyte (LGL) leukemia, human T-cell leukemia virus type 1-positive (HTLV-1+) adult T-cell leukemia/lymphoma (ATL), T-cell prolymphocytic leukemia (T-PLL), and various peripheral T-cell lymphomas (PTCLs), including but not limited to angioimmunoblastic T-cell lymphoma (AITL), ALK positive anaplastic large cell lymphoma, and ALK-negative anaplastic large cell lymphoma.

Suitable CAR antigens can also include antigens found on the surface of a multiple myeloma cell, i.e., a malignant plasma cell, such as BCMA, CS1, CD38, and CD19.

Alternatively, the CAR may be designed to express the extracellular portion of the APRIL protein, the ligand for BCMA and TACI, effectively co-targeting both BCMA and TACI for the treatment of multiple myeloma.

For instance, by way of non-limiting example, CD2, CD3c, CD4, CD5, CD7, TRAC, TCRβ, BCMA, CS1, CD38, and CD19 may be antigens expressed on a malignant T cell. In some embodiments, a CAR-T cell of the present disclosure comprises an extracellular domain of a chimeric antigen receptor that specifically binds to CD2. In some embodiments, a CAR-T cell of the present disclosure comprises an extracellular domain of a chimeric antigen receptor that specifically binds to CD3c. In some embodiments, a CAR-T cell of the present disclosure comprises an extracellular domain of a chimeric antigen receptor that specifically binds to CD4. In some embodiments, a CAR-T cell of the present disclosure comprises an extracellular domain of a chimeric antigen receptor that specifically binds to CD5. In yet some embodiments, a CAR-T cell of the present disclosure comprises an extracellular domain of a chimeric antigen receptor that specifically binds to CD7. In yet some embodiments, a CAR-T cell of the present disclosure comprises an extracellular domain of a chimeric antigen receptor that specifically binds to TRAC. In yet some embodiments, a CAR-T cell of the present disclosure comprises an extracellular domain of a chimeric antigen receptor that specifically binds to TCRβ. In still some embodiments, a CAR-T cell of the present disclosure comprises an extracellular domain of a chimeric antigen receptor that specifically binds to BCMA. In still some embodiments, a CAR-T cell of the present disclosure comprises an extracellular domain of a chimeric antigen receptor that specifically binds to CS1. In still some embodiments, a CAR-T cell of the present disclosure comprises an extracellular domain of a chimeric antigen receptor that specifically binds to CD38. In still yet some embodiments, a CAR-T cell of the present disclosure comprises an extracellular domain of a chimeric antigen receptor that specifically binds to CD19.

A chimeric antigen receptor of the present disclosure also comprises an intracellular domain that provides an intracellular signal to the T cell upon antigen binding to the antigen-specific extracellular domain. The intracellular signaling domain of a chimeric antigen receptor of the present disclosure is responsible for activation of at least one of the effector functions of the T cell in which the chimeric receptor is expressed.

The term “intracellular domain” refers to the portion of a CAR that transduces the effector function signal upon binding of an antigen to the extracellular domain and directs the T cell to perform a specialized function. Non-limiting examples of suitable intracellular domains include the zeta chain of the T-cell receptor or any of its homologs (e.g., eta, delta, gamma, or epsilon), MB 1 chain, 829, FcRIII, FcRI, and combinations of signaling molecules, such as CD3.zeta. and CD28, CD27, 4-1 BB, DAP-1 0, OX40, and combinations thereof, as well as other similar molecules and fragments. Intracellular signaling portions of other members of the families of activating proteins may be used, such as FcγRIII and FcεRI. While usually the entire intracellular domain will be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term intracellular domain is thus meant to include any truncated portion of the intracellular domain sufficient to transduce the effector function signal. Typically, the antigen-specific extracellular domain is linked to the intracellular domain of the chimeric antigen receptor by a transmembrane domain. A transmembrane domain traverses the cell membrane, anchors the CAR to the T cell surface, and connects the extracellular domain to the intracellular signaling domain, thus impacting expression of the CAR on the T cell surface. Chimeric antigen receptors may also further comprise one or more costimulatory domain and/or one or more spacer. A costimulatory domain is derived from the intracellular signaling domains of costimulatory proteins that enhance cytokine production, proliferation, cytotoxicity, and/or persistence in vivo. A “peptide hinge” connects the antigen-specific extracellular domain to the transmembrane domain. The transmembrane domain is fused to the costimulatory domain, optionally a costimulatory domain is fused to a second costimulatory domain, and the costimulatory domain is fused to a signaling domain, not limited to CD3ζ. For example, inclusion of a spacer domain between the antigen-specific extracellular domain and the transmembrane domain, and between multiple scFvs in the case of tandem CAR, may affect flexibility of the antigen-binding domain(s) and thereby CAR function. Suitable transmembrane domains, costimulatory domains, and spacers are known in the art. In a similar manner, other mono CAR-T cells may be constructed.

The CAR-T cells encompassed by the present disclosure are deficient in one or more antigens to which the chimeric antigen receptor specifically binds and are therefore fratricide-resistant. In some embodiments, the one or more antigens of the T cell is modified such the chimeric antigen receptor no longer specifically binds the one or more modified antigens. For example, the epitope of the one or more antigens recognized by the chimeric antigen receptor may be modified by one or more amino acid changes (e.g., substitutions or deletions) or the epitope may be deleted from the antigen. In some embodiments, expression of the one or more antigens is reduced in the T cell by at least 50%, at least 60%, at least 70%, at least 80%, at least 90% or more. Methods for decreasing the expression of a protein are known in the art and include, but are not limited to, modifying or replacing the promoter operably linked to the nucleic acid sequence encoding the protein. In still some embodiments, the T cell is modified such that the one or more antigens is not expressed, e.g., by deletion or disruption of the gene encoding the one or more antigens. In each of the above embodiments, the CAR-T cell may be deficient in one or preferably all the antigens to which the chimeric antigen receptor specifically binds. Methods for genetically modifying a T cell to be deficient in one or more antigens are well known in art. In an exemplary embodiment, CRISPR/cas9 gene editing can be used to modify a T cell to be deficient in one or more antigens.

CAR-T cells encompassed by the present disclosure may further be deficient in endogenous T cell receptor (TCR) signaling as a result of deleting a part of the T Cell Receptor (TCR)-CD3 complex. In various embodiments it may be desirable to eliminate or suppress endogenous TCR signaling in CAR-T cells disclosed herein. For example, decreasing or eliminating endogenous TCR signaling in CAR-T cells may prevent or reduce graft versus host disease (GvHD) when allogenic T cells are used to produce the CAR-T cells. Methods for eliminating or suppressing endogenous TCR signaling are known in the art and include, but are not limited to, deleting a part of the TCR-CD3 receptor complex, e.g., the TCR receptor alpha chain (TRAC), the TCR receptor beta chain (TRBC), CD3.epsilon, CD3.gamma, CD3.delta, and/or CD3.gamma. Deleting a part of the TCR receptor complex may block TCR mediated signaling and may thus permit the safe use of allogeneic T cells as the source of CAR-T cells without inducing life-threatening GvHD.

Alternatively, or in addition, CAR-T cells encompassed by the present disclosure may further comprise one or more suicide genes. As used herein, “suicide gene” refers to a nucleic acid sequence introduced to a CAR-T cell by standard methods known in the art that, when activated, results in the death of the CAR-T cell. Suicide genes may facilitate effective tracking and elimination of the CAR-T cells in vivo if required. Facilitated killing by activating the suicide gene may occur by methods known in the art. Suitable suicide gene therapy systems known in the art include, but are not limited to, various the herpes simplex virus thymidine kinase (HSVtk)/ganciclovir (GCV) suicide gene therapy systems or inducible caspase 9 protein. In an exemplary embodiment, a suicide gene is a CD34/thymidine kinase chimeric suicide gene.

A genome-edited, dual CAR-T cell, i.e., CD2*CD3e-dCARTΔCD2ΔCD3c, may be generated by cloning a commercially synthesized anti-CD2 single chain variable fragment into a lentiviral vector containing a 3rd generation CAR backbone with CD28 and 4-1BB internal signaling domains and cloning a commercially synthesized anti-CD3e single chain variable into the same lentiviral vector containing an additional 3rd generation CAR backbone with CD28 and 4-1BB internal signaling domains resulting in a plasmid from which the two CAR constructs are expressed from the same vector.

In some embodiments, the disclosure provides an engineered T cell comprising a dual Chimeric Antigen Receptor (dCAR), i.e., two CARs expressed from a single lentivirus construct, that specifically binds CD5 and TCR receptor alpha chain (TRAC), wherein the T cell is deficient in CD5 and TRAC (e.g., CD5*TRAC-dCARTΔCD5ΔTRAC cell). In non-limiting examples the deficiency in CD5 and the TCR receptor alpha chain (TRAC) resulted from (a) modification of CD5 and the TCR receptor alpha chain (TRAC) expressed by the T cell such that the chimeric antigen receptor no longer specifically binds the modified CD5 and the TCR receptor alpha chain (TRAC), (b) modification of the T cell such that expression of the CD5 and the TCR receptor alpha chain (TRAC) is reduced in the T cell by at least 50%, at least 60%, at least 70%, at least 80%, at least 90% or more, or (c) modification of the T cell such that CD5 and the TCR receptor alpha chain (TRAC) is not expressed (e.g., by deletion or disruption of the gene encoding CD5 and/or the TCR receptor alpha chain (TRAC). In further embodiments, the T cell comprises a suicide gene. In non-limiting examples the suicide gene expressed in the CD5*TRAC-CARTΔCD5ΔTRAC cells encodes a modified Human-Herpes Simplex Virus-1-thymidine kinase (TK) gene fused in-frame to the extracellular and transmembrane domains of the human CD34 eDNA.

In a second embodiment, the disclosure provides an engineered T cell compromising a dCAR that specifically binds CD7 and TCR receptor alpha chain (TRAC), wherein the T cell is deficient in CD7 and TRAC (e.g., CD7*TRAC-dCARTΔCD7ΔTRAC cell). In non-limiting examples the deficiency in CD7 and the TCR receptor alpha chain (TRAC) resulted from (a) modification of CD5 and the TCR receptor alpha chain (TRAC) expressed by the T cell such that the chimeric antigen receptor no longer specifically binds the modified CD7 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD7 and the TCR receptor alpha chain (TRAC) is not expressed (e.g., by deletion or disruption of the gene encoding CD7 and/or the TCR receptor alpha chain (TRAC). In further embodiments, the T cell comprises a suicide gene. In non-limiting examples the suicide gene expressed in the CD7*TRAC-dCARTΔCD7ΔTRAC cells encodes a modified Human-Herpes Simplex Virus-1-thymidine kinase (TK) gene fused in-frame to the extracellular and transmembrane domains of the human CD34 eDNA.

In a third embodiment, the disclosure provides an engineered T cell compromising a dCAR that specifically binds CD2 and TCR receptor alpha chain (TRAC), wherein the T cell is deficient in CD2 and TRAC (e.g., CD2*TRAC-dCARTΔCD2ΔTRAC cell). In non-limiting examples the deficiency in CD2 and the TCR receptor alpha chain (TRAC) resulted from (a) modification of CD2 and the TCR receptor alpha chain (TRAC) expressed by the T cell such that the chimeric antigen receptor no longer specifically binds the modified CD2 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD2 and the TCR receptor alpha chain (TRAC) is not expressed (e.g., by deletion or disruption of the gene encoding CD2 and/or the TCR receptor alpha chain (TRAC). In further embodiments, the T cell comprises a suicide gene. In non-limiting examples the suicide gene expressed in the CD2*TRAC-dCARTΔCD2ΔTRAC cells encodes a modified Human-Herpes Simplex Virus-1-thymidine kinase (TK) gene fused in-frame to the extracellular and transmembrane domains of the human CD34 eDNA.

In a similar manner, other dual CAR-T cells may be constructed.

A tandem CAR-T cell (equivalently, tCAR-T), is a T cell with a single chimeric antigen polypeptide containing two distinct antigen recognition domains with affinity to different targets wherein the antigen recognition domains are linked through a peptide linker and share common costimulatory domain (s), wherein binding of either antigen recognition domain will signal though a common costimulatory domains(s) and signaling domain.

In some embodiments, the disclosure provides an engineered T cell comprising a tandem Chimeric Antigen Receptor (tCAR), i.e., two scFv sharing a single intracellular domain, that specifically binds CD5 and TCR receptor alpha chain (TRAC), wherein the T cell is deficient in CD5 and TRAC (e.g., CD5*TRAC-tCARTΔCD5ΔTRAC cell). In non-limiting examples the deficiency in CD5 and the TCR receptor alpha chain (TRAC) resulted from (a) modification of CD5 and the TCR receptor alpha chain (TRAC) expressed by the T cell such that the chimeric antigen receptor no longer specifically binds the modified CD5 and the TCR receptor alpha chain (TRAC), (b) modification of the T cell such that expression of the CD5 and the TCR receptor alpha chain (TRAC) is reduced in the T cell by at least 50%, at least 60%, at least 70%, at least 80%, at least 90% or more, or (c) modification of the T cell such that CD5 and the TCR receptor alpha chain (TRAC) is not expressed (e.g., by deletion or disruption of the gene encoding CD5 and/or the TCR receptor alpha chain (TRAC). In further embodiments, the T cell comprises a suicide gene. In non-limiting examples the suicide gene expressed in the CD5*TRAC-tCARTΔCD5ΔTRAC cells encodes a modified Human-Herpes Simplex Virus-1-thymidine kinase (TK) gene fused in-frame to the extracellular and transmembrane domains of the human CD34 eDNA.

In a second embodiment, the disclosure provides an engineered T cell compromising a tCAR that specifically binds CD7 and TCR receptor alpha chain (TRAC), wherein the T cell is deficient in CD7 and TRAC (e.g., CD7*TRAC-tCARTΔCD7ΔTRAC cell). In non-limiting examples the deficiency in CD7 and the TCR receptor alpha chain (TRAC) resulted from (a) modification of CD5 and the TCR receptor alpha chain (TRAC) expressed by the T cell such that the chimeric antigen receptor no longer specifically binds the modified CD7 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD7 and the TCR receptor alpha chain (TRAC) is not expressed (e.g., by deletion or disruption of the gene encoding CD7 and/or the TCR receptor alpha chain (TRAC). In further embodiments, the T cell comprises a suicide gene. In non-limiting examples the suicide gene expressed in the CD7*TRAC-tCARTΔCD7ΔTRAC cells encodes a modified Human-Herpes Simplex Virus-1-thymidine kinase (TK) gene fused in-frame to the extracellular and transmembrane domains of the human CD34 eDNA.

In a third embodiment, the disclosure provides an engineered T cell compromising a tCAR that specifically binds CD2 and TCR receptor alpha chain (TRAC), wherein the T cell is deficient in CD2 and TRAC (e.g., CD2*TRAC-tCARTΔCD2ΔTRAC cell). In non-limiting examples the deficiency in CD2 and the TCR receptor alpha chain (TRAC) resulted from (a) modification of CD2 and the TCR receptor alpha chain (TRAC) expressed by the T cell such that the chimeric antigen receptor no longer specifically binds the modified CD2 and the TCR receptor alpha chain (TRAC), (b) modification of the T cell such that expression of the CD7 and the TCR receptor alpha chain (TRAC) is reduced in the T cell by at least 50%, at least 60%, at least 70%, at least 80%, at least 90% or more, or (c) modification of the T cell such that CD2 and the TCR receptor alpha chain (TRAC) is not expressed (e.g., by deletion or disruption of the gene encoding CD2 and/or the TCR receptor alpha chain (TRAC). In further embodiments, the T cell comprises a suicide gene. In non-limiting examples the suicide gene expressed in the CD2*TRAC-tCARTΔCD2ΔTRAC cells encodes a modified Human-Herpes Simplex Virus-1-thymidine kinase (TK) gene fused in-frame to the extracellular and transmembrane domains of the human CD34 eDNA.

In a similar manner, other tandem CAR-T cells may be constructed.

In certain embodiments, the disclosure provides an engineered iNKT cell comprising a single CAR, that specifically binds CD7, wherein the iNKT cell is deficient in CD7 (e.g., CD7-iNKT-CARΔCD7 cell). In non-limiting examples, the deficiency in CD7 resulted from (a) modification of CD7 expressed by the iNKT cell such that the chimeric antigen receptors no longer specifically binds the modified CD7, (b) modification of the iNKT cell such that expression of CD7 is reduced in the iNKT cell by at least 50%, at least 60%, at least 70%, at least 80%, at least 90% or more, or (c) modification of the iNKT cell such that CD7 is not expressed (e.g., by deletion or disruption of the gene encoding CD7. In further embodiments, the iNKT cell comprises a suicide gene. In non-limiting examples the suicide gene expressed in the CD7-iNKT-CARΔCD7 cells encodes a modified Human-Herpes Simplex Virus-1-thymidine kinase (TK) gene fused in-frame to the extracellular and transmembrane domains of the human CD34 cDNA.

The CAR for a CD7 specific iNKT-CAR cell may be generated by cloning a commercially synthesized anti-CD7 single chain variable fragment (scFv) into a 3rd generation CAR backbone with CD28 and 4-1BB internal signaling domains. An extracellular hCD34 domain may be added after a P2A peptide to enable both detection of CAR following viral transduction and purification using anti-hCD34 magnetic beads. A similar method may be followed for making CARs specific for other malignant T cell antigens.

In a similar manner, other mono CAR-iNKT cells may be constructed.

In certain embodiments, the disclosure provides an engineered iNKT cell comprising a dual CAR (dCAR), i.e., two CARs expressed from a single lentivirus construct, that specifically binds CD7 and CD2, wherein the iNKT cell is deficient in CD7 and CD2 (e.g., CD7×CD2-iNKT-dCARΔCD7ΔCD2 cell). In non-limiting examples, the deficiency in CD7 and CD2 resulted from (a) modification of CD7 and CD2 expressed by the iNKT cell such that the chimeric antigen receptors no longer specifically binds the modified CD7 or CD2, (b) modification of the iNKT cell such that expression of CD7 and CD2 is reduced in the iNKT cell by at least 50%, at least 60%, at least 70%, at least 80%, at least 90% or more, or (c) modification of the iNKT cell such that CD7 and CD2 is not expressed (e.g., by deletion or disruption of the gene encoding CD7 and/or CD2. In further embodiments, the iNKT cell comprises a suicide gene. In non-limiting examples the suicide gene expressed in the CD7*CD2-iNKT-dCARΔCD7ΔCD2 cells encodes a modified Human-Herpes Simplex Virus-1-thymidine kinase (TK) gene fused in-frame to the extracellular and transmembrane domains of the human CD34 cDNA. In a similar manner, other dual CAR-iNKT cells may be constructed.

In certain embodiments, the disclosure provides an engineered iNKT cell comprising a tandem CAR (tCAR), i.e., two scFv sharing a single intracellular domain, that specifically binds CD7 and CD2, wherein the iNKT cell is deficient in CD7 and CD2 (e.g., CD7×CD2-iNKT-tCARΔCD7ΔCD2 cell). In non-limiting examples, the deficiency in CD7 and CD2 resulted from (a) modification of CD7 and CD2 expressed by the iNKT cell such that the chimeric antigen receptors no longer specifically binds the modified CD7 or CD2, (b) modification of the iNKT cell such that expression of CD7 and CD2 is reduced in the iNKT cell by at least 50%, at least 60%, at least 70%, at least 80%, at least 90% or more, or (c) modification of the iNKT cell such that CD7 and CD2 is not expressed (e.g., by deletion or disruption of the gene encoding CD7 and/or CD2. In further embodiments, the iNKT cell comprises a suicide gene. In non-limiting examples the suicide gene expressed in the CD7*CD2-iNKT-tCARΔCD7ΔCD2 cells encodes a modified Human-Herpes Simplex Virus-1-thymidine kinase (TK) gene fused in-frame to the extracellular and transmembrane domains of the human CD34 cDNA.

A tCAR for a genome-edited, tandem iNKT-CAR cell, i.e., CD7*CD2-iNKT-tCARΔCD7ΔCD2, may be generated by cloning a commercially synthesized anti-CD7 single chain variable fragment (scFv) and an anti-CD2 single chain variable fragment (scFv) into a 3rd generation CAR backbone with CD28 and 4-1BB internal signaling domains. An extracellular hCD34 domain may be added after a P2A peptide to enable both detection of CAR following viral transduction and purification using anti-hCD34 magnetic beads. A similar method may be followed for making tCARs specific for other malignant T cell antigens.

In a similar manner, other tandem iNKT-CARs may be constructed.

CARs may designed as disclosed in WO2018027036A1, optionally employing variations which will be known to those of skill in the art. Lentiviral vectors and cell lines can be obtained, and guide RNAs designed, validated, and synthesized, as disclosed therein as well as by methods known in the art and from commercial sources.

Engineered CARs may be introduced into T cells, iNKT cells, or NK cells using retroviruses, which efficiently and stably integrate a nucleic acid sequence encoding the chimeric antigen receptor into the target cell genome. Other methods known in the art include, but are not limited to, lentiviral transduction, transposon-based systems, direct RNA transfection, and CRISPR/Cas systems (e.g., type I, type II, or type Ill systems using a suitable Cas protein such Cas3, Cas4, Cas5, Cas5e (or CasD), Cash, Cas6e, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3,Csx17, Csx14, Csx10, Csx16, CsaX, Csx3, Csz1, Csx15, Csf1, Csf2, Csf3, Csf4, and Cu1966, etc.). Zinc finger nucleases (ZFNs) and transcription activator-like effector nucleases (TALENs) may also be used. See, e.g., Shearer RF and Saunders DN, “Experimental design for stable genetic manipulation in mammalian cell lines: lentivirus and alternatives,” Genes Cells 2015 Jan.;20(1):1-10.

### Definitions

As used herein, the terms below have the meanings indicated. Other definitions may occur throughout the specification.

When ranges of values are disclosed, and the notation “from n1 . . . to n2” or “between n1 . . . and n2” is used, where n1 and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

As used herein, the term “CD47”, “integrin-associated protein (IAP)”, “ovarian cancer antigen OA3”, “Rh-related antigen” and “MERG” are synonymous and may be used interchangeably.

The term “anti-CD47 antibody” refer to an antibody of the disclosure which is intended for use as a therapeutic agent and will possess the binding affinity required to be useful as a therapeutic agent.

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts” with or directed against is meant that the antibody reacts with one or more antigenic determinants of the desired antigen and does not react with other polypeptides or binds at a much lower affinity (Kd >10⁻⁶). Antibodies include but are not limited to, polyclonal, monoclonal, chimeric, Fab fragments, Fab′ fragments, F(ab′)2 fragments, single chain Fv fragments, and one-armed antibodies.

As used herein, the term “monoclonal antibody” (mAb) as applied to the present antibody compounds refers to an antibody that is derived from a single copy or clone including, for example, any eukaryotic, prokaryotic, or phage clone, and not the method by which it is produced. mAbs of the present disclosure preferably exist in a homogeneous or substantially homogeneous population. Complete mAbs contain 2 heavy chains and 2 light chains.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv); and multi-specific antibodies formed from antibody fragments.

As disclosed herein, “antibody compounds” refers to mAbs and antigen-binding fragments thereof. Additional antibody compounds exhibiting similar functional properties according to the present disclosure can be generated by conventional methods. For example, mice can be immunized with human CD47 or fragments thereof, resulting antibodies can be recovered and purified, and a determination of whether they possess similar/identical binding and functional properties to the antibody compounds disclosed herein can be assessed by methods known in the art. Antigen binding fragments can also be prepared by conventional methods. The methods for producing and purifying antibodies and antigen binding fragments are well known in the art and can be found, for example, in Harlow and Lane (1988) Antibodies, A Laboratory Manual, Cold Spring Harbor Laboratory Press, Cold Spring Harbor, N.Y., Chapters 5-8 and 15.

As used herein, the terms “Fc region”, “Fc fragment”, or “Fc” refers to a protein which includes the heavy chain constant region 2 (CH₂) and the heavy chain constant region 3 (CH₃) of immunoglobulin but does not include its variable regions of the heavy chain and the light chain and the light chain constant region (CL₁), and it may further include a hinge region of the heavy chain constant region. A hybrid Fc or a hybrid Fc fragment thereof may be called “hFc” or “hyFc.” concept.

As used herein, the terms “humanized” and/or “humanization”, refer to grafting of the murine monoclonal antibody CDRs disclosed herein to human frameworks (FRs) and constant regions. Also encompassed by these terms are possible further modifications to the murine CDRs, and human FRs, by the methods disclosed in Kashmiri et al. (2005) Methods 36(1):25-34 and Hou et al. (2008) J Biochem. 144(1):115-120 to improve various antibody properties.

As used herein, the term “humanized antibodies” refers to mAbs and antigen binding fragments thereof, including the anti-CD47 antibody compounds disclosed herein, that have binding and functional properties according to the disclosure and that have FRs and constant regions that are substantially human or fully human surrounding CDRs derived from a non-human antibody.

As used herein, the term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). Examples of cancer include, but are not limited to, carcinomas, lymphomas, blastomas, sarcomas, myelomas, and leukemias.

More particular examples of these cancers or malignancies are noted below and include: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease located in an organ system listed above.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “composition” as used herein refers to an immunotherapeutic cell population combination with one or more therapeutically acceptable carriers.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “effector function” refers to a specialized function of a differentiated cell. An effector function of a T cell, for example, may be cytolytic activity or helper activity including the secretion of cytokines. An effector function in a naive, memory, or memory-type T cell may also include antigen-dependent proliferation.

The term “fratricide” as used herein means a process which occurs when a CAR-T cell, an iNKT-CAR cell, or an NK-CAR cell becomes the target of, and is killed by, another CAR-T cell, iNKT-CAR cell, or NK-CAR cell comprising the same chimeric antigen receptor as the target of a CAR-T cell, an iNKT-CAR cell, or an NK-CAR cell because the targeted cell expresses the antigen specifically recognized by the chimeric antigen receptor on both cells. CAR-T cells, iNKT-CAR cells, or NK-CAR cells comprising a chimeric antigen receptor which are deficient in an antigen to which the chimeric antigen receptor specifically binds will be “fratricide-resistant.”

As used herein, the term “gene expression” or “expression” of an IL-15 protein is understood to refer to transcription of a DNA sequence, translation of an mRNA transcript, and secretion of a protein product, or an antibody, or an antibody fragment thereof.

As used herein, the term “gene expression” or “expression” of an IL-15 protein is understood to refer to transcription of a DNA sequence, translation of an mRNA transcript, and secretion of a protein product, or an antibody, or an antibody fragment thereof.

The term “genome-edited” as used herein means having a gene added, deleted, or modified to be non-functional. Thus, in certain embodiments, a “gene-edited T cell” or a “gene-edited T cell, NK cell, or iNKT cell” is a T cell, NK cell, or iNKT cell that has had a gene such as a CAR recognizing at least one antigen added; and/or has had a gene such as the gene(s) to the antigen(s) that are recognized by the CAR deleted.

A “healthy donor,” as used herein, is one who does not have a hematologic malignancy (e.g., a T-cell malignancy).

As used herein, the term “host cell” refers to a prokaryotic cell and/or a eukaryotic cell into which a recombinant expression vector can be introduced.

As used herein, the terms “hyperproliferative disease” and “hyperproliferative disorder” refer to all neoplastic cell growth and proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and “cancer.” Additional examples of hyperproliferative diseases, disorders, and/or conditions include, but are not limited to neoplasms, whether benign or malignant, located in any tissue, system, or organ of the body.

The term “immune checkpoint inhibitor” refers to a type of drug that blocks certain proteins made by some types of immune system cells, such as T cells, and some cancer cells.

The term “immune effector cell,” as used herein, are cells that are actively involved in the destruction of tumor cells, e.g., possess anti-tumor activity. These cells may include, but are not limited to, natural killer (NK) cells, cytotoxic T cells, and memory T cells.

The term “chimeric antigen receptor (CAR)-bearing immune effector cells are immune effector cells that express a chimeric antigen receptor. These cells may include, but are not limited to, CAR-T cells, CAR-bearing iNKT cells (iNKT-CAR), or CAR-bearing NK cells (NK-CAR).

The term “CAR-T cell” means a CAR-T cell that expresses a chimeric antigen receptor.

A “dual CAR-T cell” (equivalently, dCAR-T) is a CAR-T cell that expresses two distinct chimeric antigen receptor polypeptides with affinity to different target antigens expressed within the same effector cell, wherein each CAR functions independently. The CAR may be expressed from a single polynucleotide sequence or multiple polynucleotide sequences.

A tandem “CAR-T cell” (equivalently, tCAR-T) is a CAR-T cell with a single chimeric antigen polypeptide containing two distinct antigen recognition domains with affinity to different targets, wherein the antigen recognition domains are linked through a peptide linker and share common costimulatory domain(s), and wherein binding of either antigen recognition domain will signal though a common costimulatory domains(s) and signaling domain.

The term “CAR-iNKT cell” (equivalently, iNKT-CAR) means an iNKT cell that expresses a chimeric antigen receptor.

A “dual iNKT-CAR cell” (equivalently, iNKT-dCAR) is an iNKT-CAR cell that expresses two distinct chimeric antigen receptor polypeptides with affinity to different target antigens expressed within the same effector cell, wherein each CAR functions independently. The CAR may be expressed from a single polynucleotide sequence or multiple polynucleotide sequences.

A “tandem iNKT-CAR cell” (equivalently, iNKT-tCAR) is an iNKT-CAR cell with a single chimeric antigen polypeptide containing two distinct antigen recognition domains with affinity to different targets, wherein the antigen recognition domains are linked through a peptide linker and share common costimulatory domain(s), and wherein binding of either antigen recognition domain will signal though a common costimulatory domains(s) and signaling domain.

The term “CAR-NK cell” (equivalently, NK-CAR) means an NK cell that expresses a chimeric antigen receptor.

A “dual NK-CAR cell” (equivalently, NK-dCAR) is an NK-CAR cell that expresses two distinct chimeric antigen receptor polypeptides with affinity to different target antigens expressed within the same effector cell, wherein each CAR functions independently. The CAR may be expressed from a single polynucleotide sequence or multiple polynucleotide sequences.

A “tandem NK-CAR cell” (equivalently, NK-tCAR) is an NK-CAR cell with a single chimeric antigen polypeptide containing two distinct antigen recognition domains with affinity to different targets, wherein the antigen recognition domains are linked through a peptide linker and share common costimulatory domain(s), and wherein binding of either antigen recognition domain will signal though a common costimulatory domains(s) and signaling domain.

As used herein, the term “malignancy” refers to a non-benign tumor or a cancer.

The term “malignant T cell” refers to a T cell derived from a T-cell malignancy. The term “T-cell malignancy” refers to a broad, heterogeneous grouping of malignancies derived from T-cell precursors, mature T cells, or natural killer cells. Non-limiting examples of T-cell malignancies include T-cell acute lymphoblastic leukemia/lymphoma (T-ALL), T-cell large granular lymphocyte (LGL) leukemia, human T-cell leukemia virus type 1-positive (HTLV-1+) adult T-cell leukemia/lymphoma (ATL), T-cell prolymphocytic leukemia (T-PLL), and various peripheral T-cell lymphomas (PTCLs), including but not limited to angioimmunoblastic T-cell lymphoma (AITL), ALK positive anaplastic large cell lymphoma, and ALK-negative anaplastic large cell lymphoma.

As used herein, the term “modified” refers to a polypeptide or protein having the same or similar sequence and activity to IL-7 or IL-15. As used herein, a “modified IL-7” may also be used interchangeably with a “mutant IL-7”. As used herein, a “modified IL-15” may also be used interchangeably with a “mutant IL-15.”

The term “subject,” as used herein, describes an organism, including mammals such as primates, to which treatment with the compositions according to the present invention can be provided. Mammalian species that can benefit from the disclosed methods of treatment include, but are not limited to, humans; apes; chimpanzees; orangutans; monkeys; domesticated animals such as dogs and cats; livestock such as horses, cattle, pigs, sheep.

The term “patient” is generally synonymous with the term “subject” and includes all mammals including humans.

Unless otherwise specified, the terms “protein,” “polypeptide,” and “peptide” may be used as an interchangeable concept.

As used herein, the term “signal sequence,” or equivalently, “signal peptide,” refers to a fragment directing the secretion of a biologically active molecule drug and a fusion protein, and it is cut off after being translated in a host cell. The signal sequence as used herein is a polynucleotide encoding an amino acid sequence initiating the movement of the protein penetrating the endoplasmic reticulum (ER) membrane. Useful signal sequences include an antibody light chain signal sequence, e.g., antibody 14.18 (Gillies et al., J. Immunol. Meth. 1989. 125:191-202), an antibody heavy chain signal sequence, e.g., MOPC141 an antibody heavy chain signal sequence (Sakano et al., Nature, 1980.286: 676-683), and other signal sequences know in the art (e.g., see Watson et al., Nucleic Acid Research, 1984.12:5145-5164). The characteristics of signal peptides are well known in the art, and the signal peptides conventionally having 16 to 30 amino acids, but they may include more or a fewer number of amino acid residues. Conventional signal peptides consist of three regions of the basic N-terminal region, a central hydrophobic region, and a more polar C-terminal region.

The term “therapeutically acceptable” refers to substances which are suitable for use in contact with the tissues of patients without undue toxicity, irritation, and allergic response, are commensurate with a reasonable benefit/risk ratio, and/or are effective for their intended use.

The term “effective amount,” as used herein, refers to an amount that is capable of treating or ameliorating a disease or condition or otherwise capable of producing an intended therapeutic effect.

The term “therapeutically effective” is intended to qualify the amount of active ingredients used in the treatment of a disease or disorder or on the effecting of a clinical endpoint.

As described herein, administering therapeutically-effective amounts of the disclosed compositions may be achieved by a single administration, such as for example, a single injection of sufficient amount of a disclosed interleukin, anti-CD47 antibody or fragment thereof, CD47-IL-7 fusion antibody, and/or a CAR-T cell, to provide a therapeutic benefit to the patient undergoing such treatment. Alternatively, in some circumstances, it may be desirable to provide multiple, or successive administrations of the compositions, either over a relatively short, or a relatively prolonged period of time, as may be determined by the medical practitioner overseeing administering such compositions.

As used herein, the term “moiety” refers to each of two parts into which a thing is or can be divided, or a part or portion, especially a lesser share, or a distinct part of a large molecule.

As used herein, the terms “transduced,” “transformed,” and “transfected” refer to the introduction of a nucleic acid (e.g., a vector) into a cell using a technology known in the art.

As used herein, the terms “tumor” or “tumor tissue” refer to an abnormal mass of tissue that results from excessive cell division. A tumor or tumor tissue comprises “tumor cells,” which are neoplastic cells with abnormal growth properties and no useful bodily function. Tumors, tumor tissue and tumor cells may be benign or malignant. A tumor or tumor tissue may also comprise “tumor-associated non-tumor cells,” e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “vector” is understood as a nucleic acid means which includes a nucleotide sequence that can be introduced into a host cell to be recombined and inserted into the genome of the host cell, or spontaneously replicated as an episome. The vector may include linear nucleic acids, plasmids, phagemids, cosmids, RNA vectors, virus vectors, and analogs thereof. Examples of the virus vectors may include retroviruses, adenoviruses, and adeno-associated viruses, but are not limited thereto.

### Examples

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1—Design of IL-7 Constructs

The aim of the following Examples was to generate and structurally/functionally characterize point mutants of human IL-7.

As set forth below, nine IL-7 mutants (Table 1) were generated, and their activity was tested and compared to wild-type (WT) IL-7, both commercially-sourced and generated in-house.

The IL-7 constructs were designed against the IL-7/IL-7Rα crystal structure and designed to include a C-terminal tag. The gene sequence of IL-7 wild type or mutants was custom synthesized by GenScript with a Ncol site at the N-terminus prior to the start codon. At the C-terminus, a TEV (Tobacco Etch Virus) protease cleavage site ENLYFQG and 6×His sequence was added prior to the stop codon and XhoI site. The sequence was then cloned into expression vector pET-28a(+)-TEV using standard molecular biology procedures by GenScript. Transformation and small-scale expression in Rosetta2(DE3) Singles Competent cells (Millipore Sigma, 71400) was carried out using manufacturer-supplied protocols. This expression system may be used to produce IL-7 and IL-7 mutants disclosed herein.

IL-7 mutants 51 through S5 were expressed, and purified protein from mutants 51 and S5 were obtained. For IL-7 mutants S6 through S9, expression was confirmed in small-scale induction, which can be scaled up to generate material for protein purification. IL-7 commercially sourced (GenScript) protein was produced in CHO cells.

The table below summarizes the mutants expressed and their activity in assays set forth below.

TABLE 1
IL-7 Point Mutant Summary

| | | hPBMC | Proliferation | Receptor | SEQ |
|---|---|---|---|---|---|
| | | pSTAT5 | 2E8 Cells | Binding kD | ID |
| Mutant | Mutation | EC50 (pM) | EC50 (pM) | (nM)¹ | NO: |
| IL-7 WT | — | 1.4-6.3 | 13-20 | 21.7 | 27 |
| (commercial- | | | | | |
| sourced) | | | | | |
| IL-7 WT (in- | — | 3.2 +/− 2.99 | 49.6 +/− 20.7 | 17 | 28 |
| house with | | | | | |
| His tag) | | | | | |
| IL-7 S1 with | K10I | 2.8 +/− 0.93 | 72.4 +/− 21.3 | 55 | 17 |
| C-terminal | | | | | |
| His-tag | | | | | |
| IL-7 S2 with | K10M | — | — | — | 18 |
| C-terminal | | | | | |
| His-tag | | | | | |
| IL-7 S3 with | Q11R | — | — | — | 19 |
| C-terminal | | | | | |
| His-tag | | | | | |
| IL-7 S4 with | S14T | — | — | — | 20 |
| C-terminal | | | | | |
| His-tag | | | | | |
| IL-7 S5 with | S19Q | 12.2 +/− 3.61 | 237.3 +/− 48.6 | — | 21 |
| C-terminal | | | | | |
| His-tag | | | | | |
| IL-7 S6 with | K10V | — | — | — | 22 |
| C-terminal | | | | | |
| His-tag | | | | | |
| IL-7 S7 with | K81M | — | — | — | 23 |
| C-terminal | | | | | |
| His-tag | | | | | |
| IL-7 S8 with | K81R | — | — | — | 24 |
| C-terminal | | | | | |
| His-tag | | | | | |
| IL-7 S9 with | G85M | — | — | — | 25 |
| C-terminal | | | | | |
| His-tag | | | | | |

¹Receptor binding data is from the TR-FRET assay.

### Example 2—Production of IL-7 Mutants in E. coli

Nine IL-7 mutants were identified and selected for synthesis.

IL-7 expression in E. coli was demonstrated first in small-scale culture using 2 mL of IPTG-induced culture. The culture was pelleted and lysed using 200 μl of BugBuster master mix buffer. The insoluble part after lysis was combined with 50 μl of LDS sample buffer and 10 μl reducing agent. Expression of the IL-7 mutants was then tested in scale-up culture using a 1-mL culture from a 1-L IPTG-induced culture using 200 μL of BugBuster master mix buffer for lysis and 50 μl of LDS sample buffer and 10 μl reducing agent for the insoluble part after lysis. The cell pellet was then ready for protein purification.

### Example 3—Pre-Optimized Scale Up Expression of IL-7

From a fresh transformation plate, a single E. coli colony was inoculated into 100 ml TB (Terrific broth) with appropriate antibiotics, and the culture was placed in a shaking incubator at 250 rpm and 37° C. overnight. The next morning, the culture was added to 900 ml fresh TB in a 4 L flask and grown at 37° C. until the absorbance at 600 nm (A600) reaches 0.6-0.8. IPTG (Isopropyl β-D-1-thiogalactopyranoside) was then added to a final concentration of 0.5 mM and continued to incubate for 3-5 hr. The cells were harvested by centrifuging at 9,000×g for 10 min at 4° C. The supernatant was discarded and the pellet was kept at −80° C. before purification. This expression system may be used to produce other IL-7 mutants disclosed herein.

### Example 4—ExpiCHO Expression of Human IL-7

The ExpiCHO expression system kit (ThermoFisher #A29133) was used to express human IL-7 protein. One vial of ExpiCHO-S cells was thawed and added to 125 ml vent-cap shaker flask containing 30 ml pre-warmed ExpiCHO expression medium. The cells were incubated in a 37° C. incubator with >80% relative humidity and 8% CO₂ on an orbital shaker platform with shake speed at 130 rpm (19-mm shaking diameter) for 3 days. Cell density and viability were monitored, and cells were subcultured when they reached 4×10⁶-6×10⁶ viable cells/ml at day 3 or day 4. After 3 of subcultures, the cells were split into 3×10⁶-4×10⁶ cells/ml before transfection (Day −1). On the day of transfection (Day 0), the density of cells reached 7×10⁶-1×10⁷ viable cells/ml, and viability was 95-99%. Cells were diluted to a final density of 6×10⁶ viable cells/mL with pre-warmed fresh ExpiCHO™ expression medium, and flasks were swirled gently to mix the cells. To prepare ExpiFectamine™ CHO/plasmid DNA complexes, 80 μl of 1 mg/ml IL-7 expression plasmid was added to 4 ml of OptiPRO medium in a 1.5 ml sterile microfuge tube and mixed by inversion. 640 μl of ExpiFectamine CHO reagent was added to 7.36 ml OptiPRO medium in another sterile microfuge tube and mixed by inversion. The 4 ml of diluted ExpiFectamine CHO reagent then was added to diluted IL-7 and mixed by inversion. The ExpiFectamine CHO reagent/plasmid DNA complexes were incubated for 1-5 minutes at room temperature, and then 2 ml complexes were slowly transferred to the shake flasks, and flasks were gently swirled during the transfer. The flasks were returned to 37° C. incubator with >80% relative humidity and 8% CO₂ on an orbital shaker platform with shake speed at 130 rpm. On the day after transfection (Day 1), ExpiFectamine™ CHO Enhancer and ExpiCHO™ Feed was premixed together immediately and added to the flasks for standard protocol. The flasks were gently swirled during addition and then returned to 37° C. incubator with >80% relative humidity and 8% CO₂ on an orbital shaker platform with shake speed at 130 rpm. The cells were cultured for 8-10 days, and the cell viability was monitored, and it was greater than 75% at the protein harvest. For harvest, cell culture supernatant was centrifuged at 4000-5000×g for 30 minutes in a refrigerated centrifuge, and then supernatant was filtered through a 0.22-μm filter. The supernatant was sent for purification, and expression was determined by Coomassie Blue Staining and western blot. This expression system may be used to produce other IL-7 mutants disclosed herein. Expression of IL-7 was demonstrated in the ExpiCHO expression system, but the IL-7 protein was not purified and tested. All of the IL-7 mutant constructs disclosed herein were produced and purified in the E. Coli system and tested e.g., in PSTAT, proliferation, and binding assays.

### Example 5—COS7 Cell Transient Transfections

1.0×10⁴ COS7 monkey kidney cells were plated in a volume of 100 μl RPMI 1640 medium containing 10% fetal calf serum, 100 units/ml penicillin, 100 mg/ml streptomycin, 30 mg/ml glutamine, and 50 mM 2-mercaptoethanol (complete medium) per well of a 96-well plate 18-24 hours before transfection. Cells were approximately 75% confluent on the day of transfection. For transient co-transfections of IL-7 receptor a subunit and γ subunit, 90 mL of serum-free-MEM medium was added to a well of 96-well v-bottom plate. The 500 ng of IL-7 receptor α expression plasmid (Genscript) and 500 ng of IL-7 receptor γ expression plasmid (Genscript) were added to the medium and mixed. For a 6:1 ViaFect transfection reagent (Promega #E4981): DNA ration, 6.0 μL of ViaFect was added to make a total of 100 μL volume and immediately mixed. After 5-20 minutes incubation of the ViaFect: DNA mixture at room temperature, 10 μl of ViaFect:DNA mixture per well was added to the 96-well plate containing 100 μL of cells in growth medium. The plate was mixed gently by pipetting and returned to the incubator for 24-72 hours. Co-transfection efficiency was monitor by Flow Cytometry and western blot.

### Example 6—Over-Expression of IL-7Rα & IL-2Ry in Cos-7 Cells for Binding Assay

Transfection optimization was performed, and the transfection efficiency was verified by fluorescence-activated cell sorting (FACS). Optimization parameters included the amount of plasmid to use, the ratio of plasmid vs transfection reagent, and protein expression time course.

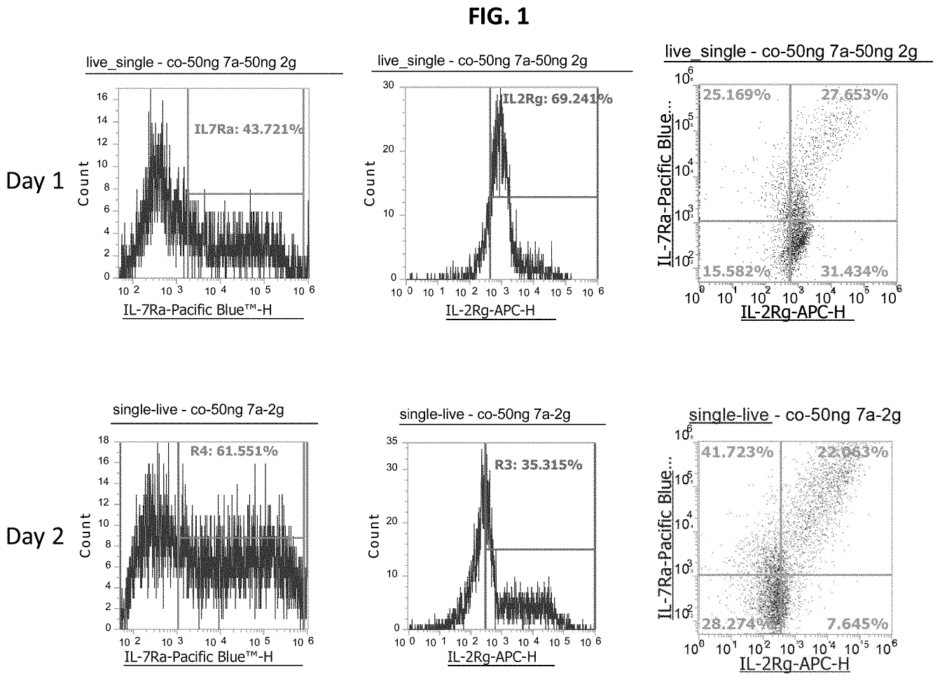

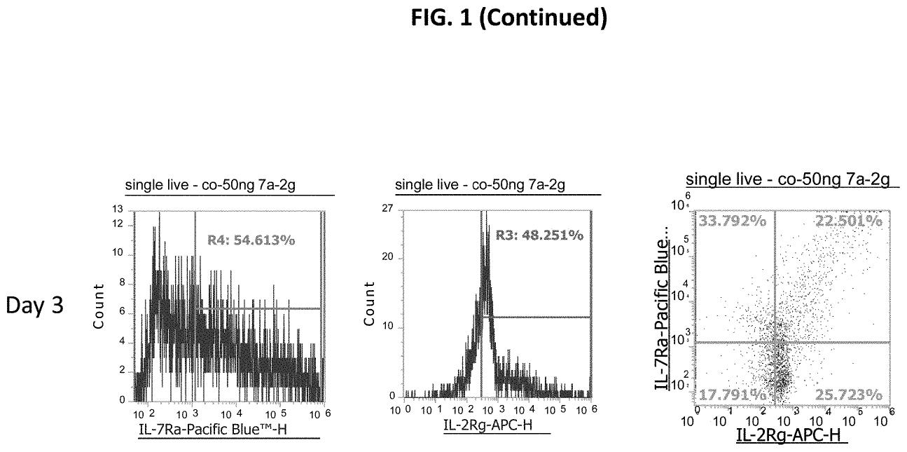

Optimization was carried out using the ViaFect transfection reagent (Promega #4981). The ratio of transfection ViaFect reagent to DNA was 6:1. Fifty (50) ng of DNA per well of a 96-well plate were used for single IL-7Rα, IL-2Rγ, or IL-2Rβ transfection, while (1)12.5 ng+12.5 ng; (2) 25 ng+25 ng; and (3) 50 ng+50 ng DNA were used for co-transfection experiments of IL-7Rα/IL-2Ry or IL-2Rβ/IL-2Rγ. FACS monitoring was performed at 1, 2, and 3 days post transfection. As shown in FIG. 1, Co-expression of IL-7Rα and IL-2Ry in Cos-7 cells produced 22% to 27% double-positive cells.

### Example 7—Refolding and Purification of IL-7 Variants

Frozen E. coli cell paste containing His6-tagged IL-7 from ˜3 L shake flask culture was thawed and suspended in lysis buffer (20 mM Tris-HCl, pH 8.0) at a ratio of 15-20 mL/g cell pellet using a tissue homogenizer. After cell suspension, 1 mM MgCl₂ was added from a 1 M MgCl₂ stock and 500 units Benzonase™ (Sigma E1014) was added. Cell lysis was carried out using and Avestin C3 instrument in two passes at 15-20,000 psi. The lysate was centrifuged at 12,000×g for 30-50 min at room temperature. The supernatant was decanted and discarded. Cell debris and the inclusion body pellet was washed with water to remove the top layer from the denser inclusion body pellet.

The pellet was suspended in 150-200 mL of 6 M guanidine-HCl, 20 mM Tris, pH 8.0, 5 mM DTT using a tissue homogenizer. To this solubilized protein solution, agarose (GoldBio H-350) was added sufficient to produce a 1-mL settled bed volume. The protein was placed on a rocking platform overnight for batch binding to agarose. The next morning, agarose beads were pelleted at 1000×g for 15 min. The supernatant was discarded and beads poured into a 1.5-cm diameter drip column (Pierce #89898). The beads were washed with 8 M urea, 20 mM Tris, pH 8.0, 1 mM DTT using 15-20 mL buffer. IL-7 protein was eluted from the column using 3.5 mL of 8 M urea buffer+200 mM imidazole (GoldBio 1-902)). Protein concentration in the eluent was calculated by A₂₈₀ from a UV absorbance spectrum using E1% of 4.30. Using this value for total protein, the volume of refolding buffer (0.1 M Tris-HCl, 0.5 M arginine-HCl, 2 mM EDTA, 0.09 mM oxidized glutathione, pH 9.0, 4° C.) was calculated to result in IL-7 protein concentration of 0.1 mg/mL. The agarose pool was added slowly to rapidly stirring refolding buffer. The solution was covered with a paper towel and placed at 4° C. for 65-90 hours. The completed refold reaction was concentrated to 4-9 mL using a 5 kD or 10 kD cutoff spin concentrator (Vivacell 100 or Amicon Ultracel). The concentrated protein was applied to a Superdex 75 size exclusion column (1.6 cm×90 cm) equilibrated and run in Dulbecco's PBS, no Ca⁺⁺, Mg⁺⁺ at 1 mL/min. Fractions of 2 mL were collected. The peak of IL-7 was identified by UV absorbance detector and typically was centered at 105 ml elution volume. This pool was concentrated to 1-2 mg/mL as determined by A₂₈₀ absorbance and stored at 4° C. These methods may be used to purify other IL-7 mutants disclosed herein.

### Example 8—Cellular Functional Analysis

JAK1,3/STAT5 signaling for IL-7 mutants was evaluated. In particular, pSTAT5 phosphorylation was evaluated in the cell lines studied, including human peripheral blood mononuclear cells (PBMCs). In addition, proliferation studies were performed for IL-7 in 2E8 cells (described below).

The protocol for evaluating JAK1,3/STAT5 signaling was as follows and as described above:

Quickly thaw frozen PBMC's and dilute cells to 10 mL in Assay Media and pellet @ 125×g for 5 minutes.

Resuspend cells in Assay Media and plate at 135 μl/well in 96-well plate (˜200,000 cells per well), v-bottom plate.

Place cell plate(s) at 37° C. while preparing cytokines.

On ice, dilute IL-7 constructs. A final dilution of 50,000 pg/mL for IL-7 was used. An 11-point curve was used, with 1:3 dilutions, plus no cytokine control.

Add 15 μL cytokine dilution to cells. Place on shaker in 37° C. incubator for 10 mins.

At 10 mins, pellet cells by centrifugation for 5 mins, for a total of 15 min. Aspirate media and lyse cells in 75 μL cold MSD lysis buffer and place on shaker at 4° C. for 30 minutes.

After completion of lysis, use undiluted lysate for analysis as per the MDS protocol for pSTAT5.

Assay Readout: MSD Multi-Spot Assay System/Phospho-STAT5a,b (Tyr694)

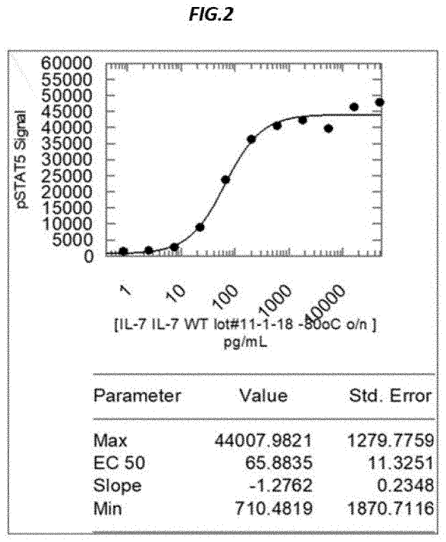

Controls/plate, IL-7:
rhIL-7 from R&D Systems, run twice
rhIL-7 WT in-house, run twice
Summaries for the data generated using the above protocol are provided in FIG. 2 and Table 2. Wild-type IL-7 obtained from commercial sources and wild-type IL-7 generated in-house in E. coli, all induced STAT5 phosphorylation as a readout of pathway activation in a concentration manner with EC50 values ranging from 24-110 pg/mL.

TABLE 2
IL-7 Signaling Data

| | EC50 | | EC50 | | Number of |
|---|---|---|---|---|---|
| | (pg/mL) | St. | (pM) | St. | Samples |
| | Average | Dev. | Average | Dev. | (n) |
| IL-7* R&D | 24 | 15 | 1.44 | 0.85 | 18 |
| IL-7* Stem Cell | 41 | 15 | 2.36 | 0.86 | 6 |
| IL-7* Miltenyi | 110 | 6 | 6.33 | 0.35 | 2 |
| IL-7 WT (in-house - | 62 | 57 | 3.22 | 2.99 | 19 |
| 110118) | | | | | |
| IL-7 WT (in-house - | 53 | 18 | 2.78 | 0.93 | 6 |
| 121818) | | | | | |
| IL-7* WT (in-house - | 323 | 70 | 19.02 | 4.12 | 4 |
| 122018) | | | | | |
| IL-7 CHO - expressed¹ | 26 | 6 | N/A | N/A | 2 |
| IL-7 S5 (in-house - | 160 | 55 | 12.22 | 3.61 | 4 |
| 01092019) | | | | | |

*Not His tagged;
¹Commercially available from Genscript.

### Example 9—Cell Proliferation Assay

Two different reagents (CCK-8 and Alamar Blue) were tested to determine the optimal conditions to assay proliferation of murine 2E8 cells in response to IL-7 stimulation. Based on the sensitivity of the two assays, Alamar Blue reagent was chosen to develop the assay further and was used for the IL-7 assay.

The cell proliferation protocol for the Alamar Blue proliferation assay in 2E8 cells for a 12-point IL-7 dose response comparing murine IL-7 (mIL-7) vs human IL-7 (hIL-7) was as follows and described further below:
1. Wash 2E8 cells 3X (DPBS) and seed at 1×10⁵ cells/mL in 96-well plate in 100 μL of medium containing no IL-7. Only the innermost 60 wells were used; the outer wells were filled with 200 μL DPBS to minimize edge effects.
2. Add 100 μL of mIL-7 or hIL-7 to give a range of concentrations from 0.0006 to 100 ng/mL, in duplicate.
3. After 72-hour incubation, add 25 μL/well Alamar Blue solution and incubate the plate at 37° C.
4. Read fluorescence (530 ex/590 em) after 20 hours.

An Alamar Blue proliferation assay was done to evaluate proliferation of 2E8 cells in response to the following preps of IL-7: (1) R&D Systems Recombinant Human IL-7 (E. coli derived); (2) Stemcell Technologies Recombinant Human IL-7 (E. coli derived); (3) Miltenyi Biotec Recombinant Human IL-7 (E. coli derived); (4) GenScript Recombinant Human IL-7, His (CHO-expressed); and (5) In-House Recombinant Human IL-7, WT, His (E. coli derived). The R&D Systems Recombinant Human IL-7 (E. coli derived) preparation had an EC50 of 0.270 pg/mL; the Stemcell Technologies Recombinant Human IL-7 (E. coli derived) preparation had an EC50 of 0.220 pg/mL; the Miltenyi Biotec Recombinant Human IL-7 (E. coli derived) preparation had an EC50 of 0.343 pg/mL; the GenScript Recombinant Human IL-7, His (CHO-expressed) preparation had an EC50 of 0.486 pg/mL; and the In-House Recombinant Human IL-7, WT, His (E. coli derived) preparation had an EC₅₀ of 0.976 pg/mL.

### Example 10—IL-7 2E8 Cell Proliferation Assay

This assay is based on the ability of IL-7 to stimulate the proliferation of IL-7-dependent immature B lymphocyte mouse cell line 2E8. 2E8 cells (ATCC TIB-239) were washed three times with DPBS (Corning #21-031-CV) and plated in white flat-bottom 96-well plates (Costar #3917) at 100,000 cells/well in 100 μL assay medium (Iscove's modified Dulbecco's medium with 4 mM L-glutamine adjusted to contain 1.5 g/L sodium bicarbonate supplemented with 0.05 mM 2-mercaptoethanol, and 20% fetal bovine serum) using only the innermost 60 wells of the plate and filling remaining wells with 200 μL DBPS to reduce issues with evaporation-related edge effects. IL-7 was then added as 100 μL of a 2X working stock (prepared as a three-fold dilution series in assay medium to 12 points, including a no IL-7 control, to give a range of final concentrations from 0.0006 to 100 ng/ml). Following a 72-hour incubation at 37° C., 25 μL of Alamar Blue reagent (ThermoFisher #DAL1025) was added per well and allowed to incubate at 37° C. for an additional 20 hours. Fluorescence (530 ex/590 em) was then measured using an LJL Analyst, and EC₅₀ was determined using Grafit software.

Binding of IL-7 produced in-house was compared to binding of commercially obtained IL-7. Initial analyses did not produce comparable conversion factors when the samples were independently analyzed. Data from one analysis was combined by having parameters for two different Kd values. The “Kd vendor” parameter was about 4-fold higher, suggesting that the rIL-7 was actually 4 times as potent as the activity of the vendor rIL-7.

### Example 11—TR-FRET Ligand-Receptor Binding Assay

IL-7 binding affinity was determined by measuring the TR-FRET fluorescence signal at a range of concentrations of recombinant IL-7 and IL-7 Receptor-alpha (IL-7Rα). In a buffer of PBS (pH 7.4)+0.05% BSA and 0.005% Tween-20, make 4×stocks of 160 nM TR-FRET acceptor linked α6-his IgG (Perkin Elmer #TRF0105-M), 80 nM TR-FRET donor linked streptavidin (Perkin Elmer #AD0062), recombinant 6-his tagged IL-7 wildtype or mutant variant (at concentrations up to 160 nM), and recombinant biotin tagged IL-7Rα (Acrobiosystems #IL-7-H82F8) (at concentrations up to 80 nM). For each sample to be tested, 5 μL of the 4× stock of a6-his IgG linked acceptor is mixed with 5 μL of a 4×working stock of IL-7. Separately, for each sample, 5 μL of 4×TR-FRET donor linked to streptavidin is mixed with 5 μL of the 4×working stock of recombinant IL-7Rα linked to biotin. Mixtures were incubated at room temperature for 30 minutes during which the 10 μL of a6-his linked acceptor/IL-7 mix was distributed into indicated wells in 384-well shallow black ProxiPlate (Perkin Elmer #6008260) and kept covered from light. After the 30 min incubation, 10 μL of streptavidin linked donor/IL-7Ra mixture was placed in designated wells containing designated concentration of IL-7 plus acceptor. Plates were immediately spun at 750 rpm for 30 sec and then immediately placed in PHERAstar plate reader (BMG Labtech) to read TR-FRET signal. Continuously over the next 30 minutes, fluorescence for donor signal (337 nM excitation/620 nM emission) and acceptor signal (337 nm excitation/655 nm emission) are read. The TR-FRET binding signal is determined by taking the ratio of acceptor signal over donor signal and multiplying by 10,000. Equilibrium is defined at the time point where the readings plateau before fluorescence activity begins to decay. Equilibrium values are then fit to a standard equilibrium equation assuming single site binding using the graphing program GraFit (Erithacus Software).

$$FRET Signal = signal constant [ Bound ] [ Bound ] = \frac{[ {Ligand}_{free} ] [ {Rec}_{free} ]}{{K}_{d}}$$

$$FRET Signal = \frac{\begin{matrix}( {K}_{d} + [ ligand ] + [ r e c ] ) - \\ \sqrt{{b ( {K}_{d} + [ ligand ] + [ r e c ] )}^{2} - 4 [ ligand ] [ r e c ]}\end{matrix}}{2 * signal constant}$$

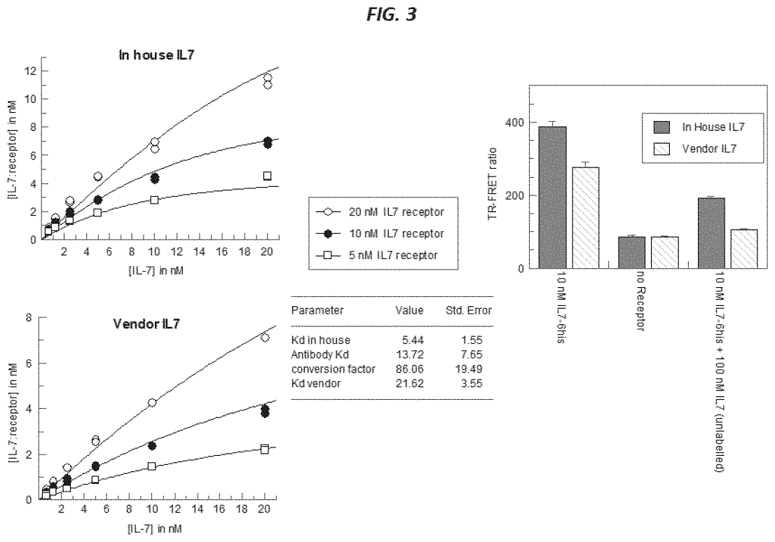

As shown in FIG. 3, the K_{d} of “in-house” IL-7 in the analysis was 5.44 nM, which is comparable to what was seen in earlier analyses (6.6 nM). The in-house IL-7 activity is only partially competed away by a 10×excess of untagged IL-7 (from R&D biosystems). The vendor activity, however, is completely competed away. It is possible that, like the Biolegends IL-7 used to measure binding, the IL-7 produced in-house is more potent than the unlabeled IL-7.

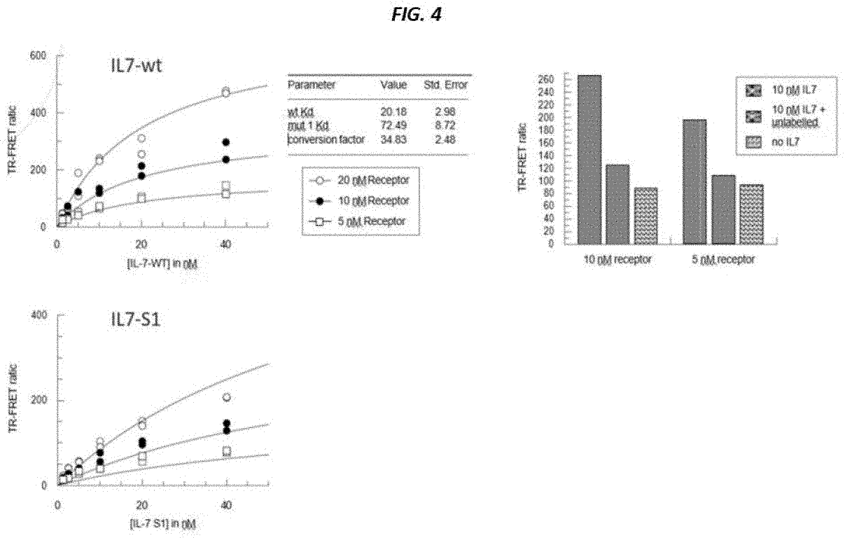

In addition, IL-7 mutant S1 was tested for binding to IL-7Rα and compared to WT. The Kd values for WT and IL-7 mutant S1 were 20.2 nM and 34.8 nM, respectively (FIG. 4). The S1 mutant appears to have a lower affinity for the receptor, and the fit does not look as clean. Of note, this analysis was done with only the α receptor present. In order to see if there are effects on receptor γ, analysis will need to be performed with this receptor chain. No complete competition was observed by a 20-fold excess of unlabeled IL-7.

### Example 12—IL-7 Binding to IL-7Rα with and without Receptor γ

Binding of in-house prepared rIL-7 was compared to binding of commercially obtained IL-7. Two sets of conditions were compared: (1) with (+) 20 nM receptor γ, and (2) without (-) receptor γ. As shown in FIG. 5, a 5-fold increase in apparent affinity was seen in the presence of the γ receptor. The measured K_{d} without the γ receptor was 12.13 nM, which was higher than previous analyses (6 nM). The increased affinity in the presence of γ receptor was expected. The model used to fit the data assumed a more simplified binding model where γ and β act as one combined unit.

Table 3 and FIG. 5 provide a summary of IL-7 receptor binding efficiencies.

TABLE 3
IL-7 Receptor Binding Efficiencies

TR-FRET (Kd in nM)

| Cytokine | Expt. 1 | Expt. 2 |
|---|---|---|
| IL-7 wt | 14 nM | 20 nM |
| IL-7 S1 | 37 nM | 72 nM |

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

### Example 13—Design of IL-15 Constructs

The aim of the following Examples was to generate and structurally/functionally characterize point mutants of human IL-15.

As set forth below, fifteen IL-15 mutants (Table 4) were generated, and their activity was tested and compared to wild-type (WT) IL-15, both commercially-sourced and generated in-house.

The IL-15 constructs were designed against the IL-15/IL-15Rα/IL-15β/γ_{c} crystal structure, and to have an N-terminal tag. The gene sequence of IL-15 wild type or mutants was custom synthesized by GenScript with NdeI site at the N-terminus and XhoI site on the C-terminus. The sequence was then cloned into expression vector pET-28a(+)-TEV using standard molecular biology procedures by GenScript so the IL-15 had a 6×His tag and N-terminal TEV protease cleavage site. Transformation and small-scale expression in One Shot™ BL21 Star™ (DE3) Chemically Competent E. coli (ThermoFisher Scientific, C601003) was carried out using manufacturer-supplied protocols. This expression system may be used to produce IL-15 and IL-15 mutants disclosed herein.

Mutants were at the IL-15Rβ and/or the γ_{c} interfaces, as the IL-15Rα affinity was already very high. Low to no recovery was found for mutations M3 and M4, respectively, but other mutants were purified and recovered in the 0.65-3.1 mg range.

The table below summarizes the mutants expressed and their activity in assays set forth below.

TABLE 4
IL-15 Point Mutant Summary

| | | hPBMC | Proliferation | Receptor | SEQ |
|---|---|---|---|---|---|
| | | pSTAT5 | MO-7E Cells | Binding Kd | ID |
| Mutant | Mutation | EC50 (pM) | EC50 (pM) | (nM)¹ | NO: |
| IL-15 WT | — | 6-16 | 20-108 | — | 61 |
| (commercial) | | | | | |
| IL-15 WT | — | 24 | — | — | — |
| (Sino Bio) | | | | | |
| IL-15 WT (in- | — | 53.7 +/− 24.3 | 876 +/− 69.9 | 0.95 +/− 0.46 | 62 |
| house His₆) | | | | | |
| IL-15 M1 | N72D | 32.4 +/− 3.6 | 232.5 +/− 55.4 | 0.62 +/− 0.25 | 31 |
| IL-15 M2 | N72R | 51(IC), | 5534 +/− 1083 | 31.36 +/− 2.71 | 32 |
| | | 31.0 +/− 3.9 | | | |
| IL-15 M3 | N72Y | 166.2 +/− 22.3 | >6494 (IC), | | 33 |
| | | | 11261.6 | | |
| IL-15 M4 | N79E | — | — | — | 34 |
| IL-15 M5 | N79S | 54.6 +/− 14.2 | 610.88 +/− 99.5 | 2.63 +/− 0.56 | 35 |
| IL-15 M6 | K11L | 205 +/− 11 | 8284.37 | 22.28 +/− 2.92 | 36 |
| IL-15 M7 | K11M | 134.4 +/− 46.8 | 3765.69 | 40.89 +/− 8.05 | 37 |
| IL-15 M8 | N112H | 123.2 +/− 12.2 | 796.38 | 19.51 +/− 2.35 | 38 |
| IL-15 M9 | N112M | 95.4 +/− 37.4 | 1982.44 | 44.24 +/− 8.15 | 39 |
| IL-15 M10 | V3I | 96.0 +/− 60.1 | 843.79 | 33.22 +/− 6.4 | 40 |
| IL-15 M11 | V3M | 177.1 +/− 54.7 | 380.9 | 12.22 +/− 1.75 | 41 |
| IL-15 M12 | V3R | 431.0 +/− 213.7 | 3098.0 +/− 2821.6 | 27.70 +/− 6.32 | 42 |
| IL-15 M13 | N4H | 206.5 +/− 43.5 | 3491.7 | 71.06 +/− 15.71 | 43 |
| IL-15 M14 | K11R | 99.7 +/− 53.9 | 7252.4 | 14.39 +/− 2.73 | 44 |
| IL-15 M15 | N112Y | 107.0 +/− 23.0 | 7344.8 | 47.04 +/− 15.93 | 45 |

¹Receptor binding data is from the TR-FRET assay.

### Example 14—Production of IL-15 Mutants in E. coli

Fifteen IL-15 mutants were identified and selected for synthesis.

For IL-15 mutants M6 through M15, IL-15 expression in E. coli was demonstrated in small-scale culture using a pellet from 1 mL of IPTG-induced culture was used with 200 μL of BugBuster master mix buffer and 35 μl of LDS sample buffer and 5 μl reducing agent for the insoluble part after lysis. Expression of the IL-15 mutants was then tested in scale-up culture using a pellet from 1 mL of IPTG-induced culture using 200 μl of BugBuster master mix buffer for lysis and 45 μl of LDS sample buffer and 5 μl reducing agent for the insoluble part after lysis.

### Example 15—Pre-Optimized Scale Up Expression of IL-15

From a fresh transformation plate, a single E. coli colony was inoculated into 100 ml TB (Terrific broth) with appropriate antibiotics, and the culture was placed in a shaking incubator at 250 rpm and 37° C. overnight. The next morning, the culture was added to 900 ml fresh TB in a 4 L flask and grown at 37° C. until the absorbance at 600 nm (A600) reaches 0.6-0.8. IPTG (Isopropyl β-D-1-thiogalactopyranoside) was then added to a final concentration of 0.5 mM and continued to incubate for 3-5 hr. The cells were harvested by centrifuging at 9,000×g for 10 min at 4° C. The supernatant was discarded and the pellet was kept at −80° C. before purification. This expression system may be used to produce other IL-15 mutants disclosed herein.

### Example 16—ExpiCHO Expression of Human IL-15

The ExpiCHO expression system kit (ThermoFisher #A29133) was used to express human IL-15 protein (IL-15 WT (in-house His₆)). One vial of ExpiCHO-S cells was thawed and added to 125 ml vent-cap shaker flask containing 30 ml pre-warmed ExpiCHO expression medium. The cells were incubated in a 37° C. incubator with >80% relative humidity and 8% CO₂ on an orbital shaker platform with shake speed at 130 rpm (19-mm shaking diameter) for 3 days. Cell density and viability were monitored, and cells were subcultured when they reached 4×10⁶-6×10⁶ viable cells/ml at day 3 or day 4. After 3 of subcultures, the cells were split into 3×10⁶-4×10⁶ cells/ml before transfection (Day −1). On the day of transfection (Day 0), the density of cells reached 7×10⁶-1×10⁷ viable cells/ml, and viability was 95-99%. Cells were diluted to a final density of 6×10⁶ viable cells/mL with pre-warmed fresh ExpiCHO™ expression medium, and flasks were swirled gently to mix the cells. To prepare ExpiFectamine™ CHO/plasmid DNA complexes, 80 μl of 1 mg/ml IL-15 expression plasmid (Genscript, pcDNA3.4 TOPO vector) was added to 4 ml of OptiPRO medium in a 1.5 ml sterile microfuge tube and mixed by inversion. 640 μl of ExpiFectamine CHO reagent was added to 7.36 ml OptiPRO medium in another sterile microfuge tube and mixed by inversion. The 4 ml of diluted ExpiFectamine CHO reagent then was added to diluted IL-15 and mixed by inversion. The ExpiFectamine CHO reagent/plasmid DNA complexes were incubated for 1-5 minutes at room temperature, and then 2 ml complexes were slowly transferred to the shake flasks, and flasks were gently swirled during the transfer. The flasks were returned to 37° C. incubator with >80% relative humidity and 8% CO₂ on an orbital shaker platform with shake speed at 130 rpm. On the day after transfection (Day 1), ExpiFectamine™ CHO Enhancer and ExpiCHO™ Feed was premixed together immediately and added to the flasks for standard protocol. The flasks were gently swirled during addition and then returned to 37° C. incubator with >80% relative humidity and 8% CO₂ on an orbital shaker platform with shake speed at 130 rpm. The cells were cultured for 8-10 days, and the cell viability was monitored, and it was greater than 75% at the protein harvest. For harvest, cell culture supernatant was centrifuged at 4000-5000×g for 30 minutes in a refrigerated centrifuge, and then supernatant was filtered through a 0.22-μm filter. The supernatant was sent for purification, and expression was determined by Coomassie Blue Staining and western blot. Expression of IL-15 was demonstrated in the ExpiCHO expression system, but the IL-15 protein was not purified and tested. All of the IL-15 mutant constructs disclosed herein were produced and purified in the E. Coli system and tested e.g., in PSTAT, proliferation, and binding assays.

### Example 17—Production of IL-15 in Chinese Hamster Ovary (CHO) Cells

When synthesizing certain proteins, glycosylation and correct disulfide bond formation for the partner construct can be important, which requires also expressing the identified mutants in a mammalian expression system (CHO). Evaluation of the mutant proteins in CHO was performed using the ExpiCHO™ Expression System (ThermoFisher). Samples transfected included WT IL-15 (GenScript) and the control supplied with the ExpiCHO™ Expression System (Antibody Expressing Positive Control Vector).

The protocol selected for this was a standard protocol for use with the selected Expression System, which required each cytokine to be cultured in three separate flasks. An additional antibody control was also included, for a total of 7 samples. The assay was conducted for 10 days in a volume of approximately 35 mL in a 250-mL flask, with harvest on days 8, 9, and 10.

To harvest the protein for analysis, samples were centrifuged at 4000 rpm for 30 minutes at 4° C. The supernatant was filtered and stored at 4° C., and the pellet was discarded.

The samples selected for synthesis included WT IL-15 (GenScript) harvested on Day 8; WT IL-15 (GenScript) harvested on Day 9; and WT IL-15 (GenScript) harvested on Day 10. Western blot analysis was also conducted on the samples from Day 8, along with +Antibody Control. Good expression of IL-7 was confirmed for samples in ExpiCHO™ medium; cultures harvested on day 8 exhibited a brighter band and therefore were expressed more in CHO cells. Expression of IL-15 was also confirmed, although the IL-15 band intensity was low, and the size was larger than expected. This expression system and analytical protocol may be used to produce other IL-15 mutants disclosed herein.

### Example 18—COS-7 Cell Transient Transfections

1.0×10⁴ COST monkey kidney cells were plated in a volume of 100 μl RPMI 1640 medium containing 10% fetal calf serum, 100 units/ml penicillin, 100 mg/ml streptomycin, 30 mg/ml glutamine, and 50 mM 2-mercaptoethanol (complete medium) per well of a 96-well plate 18-24 hours before transfection. Cells were approximately 75% confluent on the day of transfection. For transient co-transfections of IL-15 receptor β subunit and γ subunit, 90 mL of serum-free-MEM medium was added to a well of 96-well v-bottom plate. The 500 ng of IL-15 receptor β expression plasmid (Genscript) and 500 ng of IL-15 receptor γ expression plasmid (Genscript) were added to the medium and mixed. For a 6:1 ViaFect transfection reagent (Promega #E4981): DNA ration, 6.0 μL of ViaFect was added to make a total of 100 μL volume and immediately mixed. After 5-20 minutes incubation of the ViaFect: DNA mixture at room temperature, 10 μl of ViaFect:DNA mixture per well was added to the 96-well plate containing 100 μL of cells in growth medium. The plate was mixed gently by pipetting and returned to the incubator for 24-72 hours. Co-transfection efficiency was monitor by Flow Cytometry and Western-Blot.

### Example 19—Refolding and Purification of IL-15 Variants

Frozen E. coli cell paste containing His6-tagged IL-15 from ˜3 L shake flask culture was thawed and suspended in lysis buffer (20 mM Tris-HCl, pH 8.0) at a ratio of 15-20 mL/g cell pellet using a tissue homogenizer. After cell suspension, 1 mM MgCl₂ was added from a 1 M MgCl₂ stock and 500 units Benzonase™ (Sigma E1014) was added. Cell lysis was carried out using and Avestin C3 instrument in two passes at 15-20,000 psi. The lysate was centrifuged at 12,000×g for 30-50 min at room temperature. The supernatant was decanted and discarded. Cell debris and the inclusion body pellet was washed with water to remove the top layer from the denser inclusion body pellet.

The pellet was suspended in 150-200 mL of 6 M guanidine-HCl, 20 mM Tris, pH 8.0, 5 mM DTT using a tissue homogenizer. To this solubilized protein solution, agarose (GoldBio H-350) was added sufficient to produce a 1-mL settled bed volume. The protein was placed on a rocking platform overnight for batch binding to agarose. The next morning, agarose beads were pelleted at 1000× g for 15 min. The supernatant was discarded and beads poured into a 1.5-cm diameter drip column (Pierce #89898). The beads were washed with 8 M urea, 20 mM Tris, pH 8.0, 1 mM DTT using 15-20 mL buffer. IL-15 protein was eluted from the column using 3.5 mL of 8 M urea buffer+200 mM imidazole (GoldBio 1-902)). Protein concentration in the eluent was calculated by A₂₈₀ from a UV absorbance spectrum using E1% of 5.77. Using this value for total protein, the volume of refolding buffer (0.1 M Tris-HCl, 0.5 M glycine, 1 mM oxidized glutathione, 10 mM reduced glutathione, pH 8.0, 4° C.) was calculated to result in IL-15 protein concentration of 0.1 mg/mL. The Ni⁺⁺ agarose pool was added slowly to rapidly stirring refolding buffer. The solution was covered with a paper towel and placed at 4° C. for 65-90 hours. The completed refold reaction was concentrated to 4-9 mL using a 5 kD or 10 kD cutoff spin concentrator (Vivacell 100 or Amicon Ultracel). The concentrated protein was applied to a Superdex 75 size exclusion column (1.6 cm×90 cm) equilibrated and run in Dulbecco's PBS, no Ca⁺⁺, Mg⁺⁺ at 1 mL/min. Fractions of 2 mL were collected. The peak of IL-15 was identified by UV absorbance detector and typically was centered at 105 ml elution volume. This pool was concentrated to 1-2 mg/mL as determined by A₂₈₀ absorbance and stored at 4° C. These methods may be used to purify other IL-15 mutants disclosed herein.

### Example 20—Activity of IL-15 and their Mutants in Human Peripheral Blood Mononuclear Cells (PBMC) pSTAT5 (Tyr694) Assay

Human Peripheral Blood Mononuclear Cells (generated in-house) were plated in V-bottom 96 well plate at 175,000 cells/well in 135 μL medium (RPMI1640 with 10% heat inactivated serum and 47 μM 2-mercaptoethanol and 10 μM HEPES). The cell plate was placed in a 37° C. incubator while the cytokine curve was prepared. The cytokines were serial diluted in a 96 well plate using cold RPMI media previously described. IL-15 (or mutant) samples were serial diluted starting at 100,000 pg/mL, diluting 1:3, 11 pt. plus zero cytokine (unstimulated). Stimulation of hPBMCs was carried out using diluted cytokines at 15 μL per well for 10 mins shaking at 37° C. Wells designated ‘unstimulated’ received 15 μL of media only. The plate was immediately sealed with a plate seal and the cells were pelleted at 400×g for 5 minutes. The media from the wells was removed using a multichannel aspirator. 75 μL of cold MSD prepared lysis buffer was immediately added to the wells. The plate was placed at 4° C. shaking for at least 30 mins. MSD kit directions (Cat #K150IGD) were followed for pSTAT5 detection. Data are shown in Table 4.

### Example 21—Cellular Functional Analysis

JAK1,3/STAT5 signaling for IL-15 mutants was evaluated. In particular, pSTAT5 phosphorylation was evaluated in the cell lines studied, including human peripheral blood mononuclear cells (PBMCs).

The protocol for evaluating JAK1,3/STAT5 signaling was as follows and as described above:
1. Quickly thaw frozen PBMC's and dilute cells to 10 mL in Assay Media and pellet @ 125×g for 5 minutes.
2. Resuspend cells in Assay Media and plate at 135 μl/well in 96-well plate (200,000 cells per well), v-bottom plate.
3. Place cell plate(s) at 37° C. while preparing cytokines.
4. On ice, dilute IL-15 constructs. A final dilution of 100,000 pg/mL for IL-15 was used. An 11-point curve was used, with 1:3 dilutions, plus no cytokine control.
5. Add 15 μL cytokine dilution to cells. Place on shaker in 37° C. incubator for 10 mins.
6. At 10 mins, pellet cells by centrifugation for 5 mins, for a total of 15 min. Aspirate media and lyse cells in 75 μL cold MSD lysis buffer and place on shaker at 4° C. for 30 minutes.
7. After completion of lysis, use undiluted lysate for analysis as per the MDS protocol for pSTAT5.
8. Assay Readout: MSD Multi-Spot Assay System/Phospho-STAT5a,b (Tyr694)
9. Controls/plate, IL-15:
   rhIL-15 from R&D Systems, run twice
   rhIL-15 WT in-house, run twice

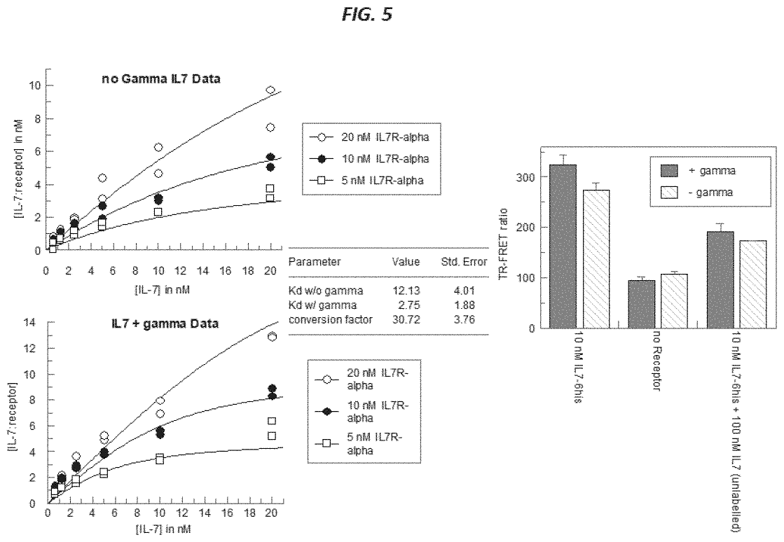

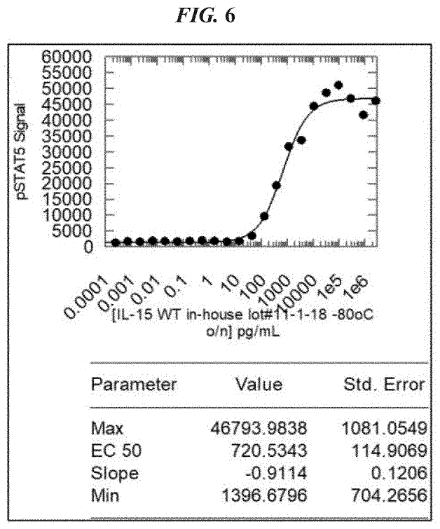

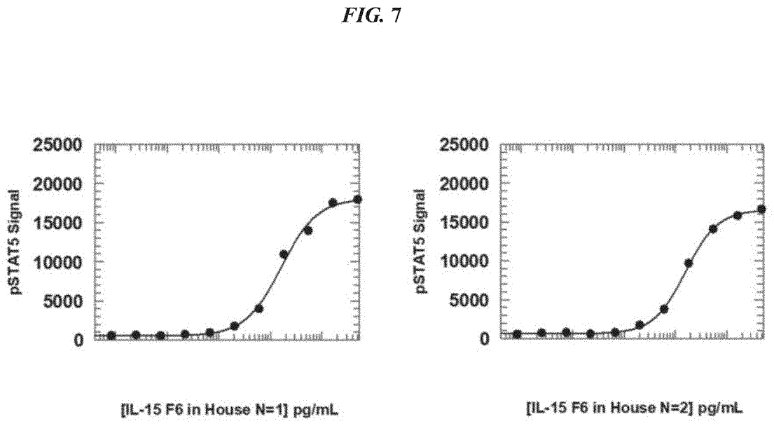

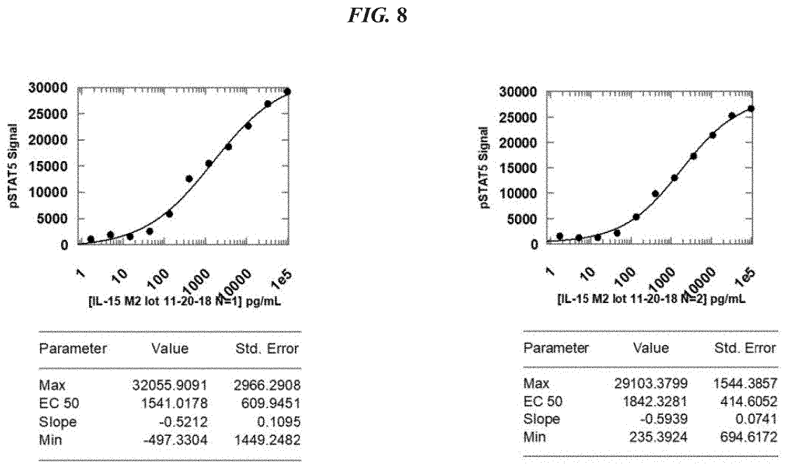

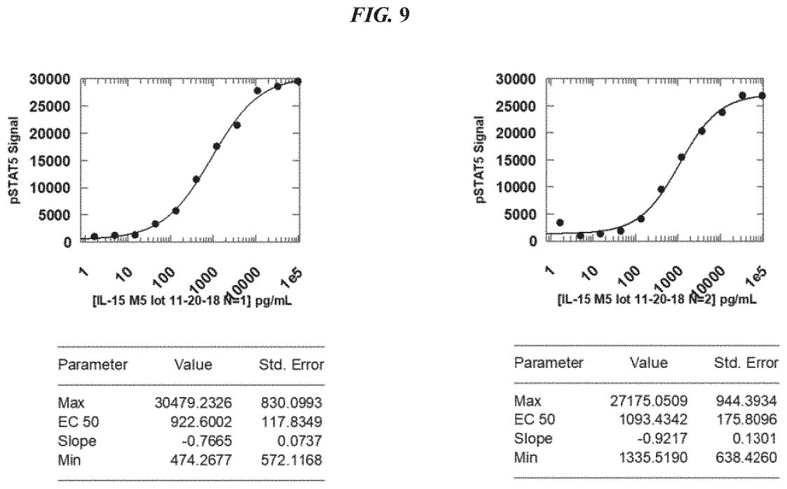

Summaries for the data generated using the above protocol are provided in Table 4 and FIG. 6. WT IL-15 SEC purified fraction 6 showed activity in a pSTAT5 assay in human PBMCs, shown in FIG. 7. In addition, example curves of cytokine activity for M2 (N72R) and M5 (N79S) are provided in FIG. 8 and FIG. 9, respectively, and Table 5 below. Wild-type IL-15 obtained from commercial sources and both wild-type and various point mutants of IL-15 generated in-house in E. coli, all induced STAT5 phosphorylation as a readout of pathway activation in a concentration manner with EC₅₀ values ranging from 76-836 pg/ml.

TABLE 5
IL-15 Signaling Data

| | EC50 (pg/mL) | St. | Number of |
|---|---|---|---|
| | Average | Dev. | Samples |
| IL-15* R&D | 199.8 | 111.0 | 16 |
| IL-15* Stem Cell | 76.2 | 10.5 | 6 |
| IL-15* Miltenyi | 177.3 | 21.3 | 2 |
| IL-15 WT in-house | 757.2 | 420.0 | 17 |
| IL-15 M2 (incomplete curves) | 780.0 | 716.5 | 7 |
| IL-15 M5 | 836.1 | 217.5 | 4 |

*Not His tagged

### Example 22—Cell Proliferation Assay

Two different reagents (CCK-8 and Alamar Blue) were tested to determine the optimal conditions to assay proliferation of murine 2E8 cells in response to IL-15 stimulation. Based on the sensitivity of the two assays, Alamar Blue reagent was chosen to develop the assay further in M-07e cells. Proliferation of M-07e cells in response to recombinant human IL-15 is described below.

The cell proliferation protocol for the Alamar Blue proliferation assay in M-07e cells for a 12-point IL-15 dose response comparing murine IL-15 (mIL-15) vs human IL-15 (hIL-15) is as follows and described further below:
5. Wash M-07e cells 3X with appropriate medium (e.g., DPBS) and seed at 1×10⁵ cells/mL in 96-well plate in 100 μL of medium containing no IL-15. Only the innermost 60 wells are used; the outer wells are filled with 200 μL DPBS to minimize edge effects.
6. Add 100 μL of mIL-15 or hIL-15 to give a range of concentrations from 0.0006 to 100 ng/mL, in duplicate.
7. After 72-hour incubation, add 25 μL/well Alamar Blue solution and incubate the plate at 37° C.
8. Read fluorescence (530 ex/590 em) after 20 hours.

### Example 23—Proliferation of M-07e Cells in Response to Recombinant Human IL-15

Activity of recombinant human IL-15 was measured in a proliferation assay using M-07e human acute megakaryoblastic leukemia cells. M-07e cells (DSMZ catalog #ACC104) were grown and sub-cultured in RPMI 1640 plus 10% heat inactivated FBS, supplemented with penicillin/streptomycin/glutamine (P/S/G) and recombinant human GM-CSF (R&D Systems catalog #215-GM-010) at 10 ng/ml. For the proliferation assay, cells were centrifuged at 150×g for 5 minutes to remove growth media containing GM-CSF. Cells were washed three times in media containing RPMI 1640 plus 10% heat inactivated FBS and P/S/G (assay media) and were then cytokine starved for 4 hours in assay media. At the end of four hours, cells were plated in a 96 well flat bottom plate at 45,000 cells/well in 150 μL assay media and recombinant human IL-15 (wild-type or various mutants) was added as 50 μL of 4×working stock solution in assay media. Cells were then placed in a 37° C., 5% CO₂ incubator for 72 hours. At the end of 72 hours, cells were centrifuged at 400×g for 5 minutes and 100 μL media was removed. Cells were lysed for 5 minutes in 100 μL CellTiter-Glow® reagent (Promega catalog #G7570). The plate was then allowed to incubate for 5-10 minutes at room temperature to stabilize luminescence signal. 50 μL lysate was transferred to an opaque-walled 96 well plate to record luminescence using a plate reader. Data are shown in Table 4.

### Example 24—IL-15 TR-FRET Ligand-Receptor Binding Assay

IL-15 binding affinity is determined by measuring the TR-FRET fluorescence signal at a range of concentrations of recombinant IL-15 and IL-2 Receptor-beta (IL-2Rβ). In a buffer of PBS (pH 7.4)+0.05% BSA & 0.005% Tween-20, make 5×stocks of 200 nM TR-FRET acceptor linked α6-his IgG (Perkin Elmer #TRF0105-M), 100 nM TR-FRET donor linked α-human IgG (Perkin Elmer #AD0074), recombinant 6-his tagged IL-15 wildtype or mutant variant (at concentrations up to 200 nM), and recombinant Fc tagged IL-2Rβ (Sino Biologicals #10696-H02H) (at concentrations up to 100 nM). For each sample to be tested, mix 4 μL of the 5× stock of a6-his IgG linked acceptor with 4 μL of a 5×working stock of IL-15. Separately, for each sample, 4 μL of 5×TR-FRET donor linked to α-human IgG is mixed with 4 μL of the 5×working stock of recombinant IL-2Rβ linked to Fc domain. Mixtures are incubated at room temperature for 30 minutes. During incubation, 5× stock of 3 μg/ml Fc block (BD Pharmingen #564220) is prepared. After 30-minute incubation, 4 μL of 5×Fc block is added to each sample of IL-2Rβ/α-human IgG and incubated at RT for another 15 minutes. During incubation, the 8 μL of a6-his linked acceptor/IL-15 mix is distributed into indicated wells in 384 well shallow black ProxiPlate (Perkin Elmer #6008260). After 15 min incubation, 12 μL of α-human IgG linked donor/IL-2Rβ/Fc block mixture is placed in designated well containing appropriate concentration of IL-15 plus acceptor. Plates are immediately spun at 750 rpm for 30 sec and then immediately placed in PHERAstar plate reader (BMG Labtech) to read TR-FRET signal. Continuously over the next 30 minutes, fluorescence for donor signal (337 nM excitation/620 nM emission) and acceptor signal (337 nm excitation/655 nm emission) are read. The TR-FRET binding signal is determined by taking the ratio of acceptor signal over donor signal and multiplying by 10,000. Equilibrium is defined at the time point where the readings plateau before fluorescence active begins to decay. Equilibrium values are then fit to a standard equilibrium equation assuming single site binding using the graphing program GraFit (Erithacus Software).

$$FRET Signal = \frac{\begin{matrix}( {K}_{d} + [ ligand ] + [ r e c ] ) - \\ \sqrt{{b ( {K}_{d} + [ ligand ] + [ r e c ] )}^{2} - 4 [ ligand ] [ r e c ]}\end{matrix}}{2 * signal constant}$$

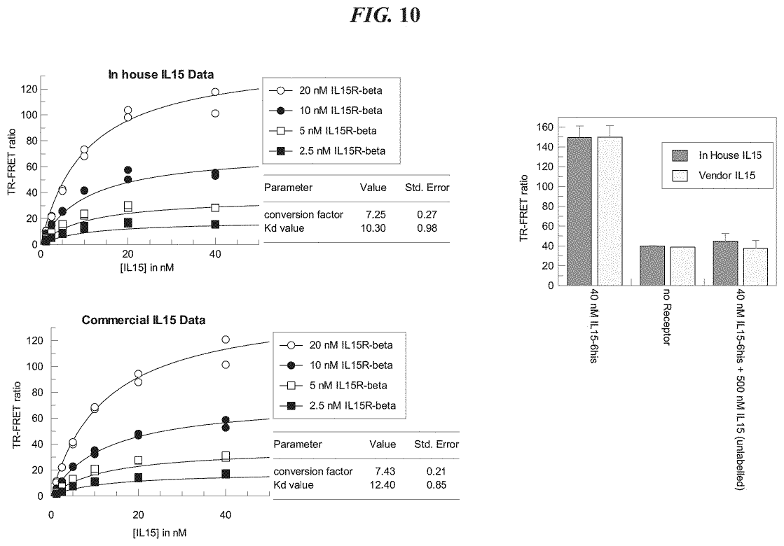

Binding of mutant IL-15 proteins to IL-15Rβ was evaluated using TR-FRET (described above). A direct comparison of commercially obtained IL-15 and in-house IL-15 in the same assay showed that recombinant IL-15 obtained from a commercial source (Sino Biologicals; Vendor IL-15) had the same activity as the recombinant IL-15 produced in-house. As shown in FIG. 10, the “conversion factor” shows the linear relationship between what the assay measures (TR-FRET ratio) and the concentration of ligand:receptor complex. In this experiment, the two sample sets had near equivalent conversion factors when independently analyzed. The most direct measure of binding activity is the Kd, and the two samples showed Kd values within 20% of one another (10.3 nM vs 12.4 nM). It was also noted that the activity was competed away by a 12.5X excess of untagged IL-15.

IL-15 mutants M2 (N72R) and M5 (N72S) were tested for binding to IL-2Rβ and compared to WT. The WT Kd was determined to be 0.95 nM, while the Kd values for mutants M2 and M5 were 31.36 nM and 2.63 nM, respectively (FIG. 11). These data suggest that mutant M5 has the same affinity as WT IL-15, while mutant M2 is about 3-fold weaker than WT. Of note, it is not able to tell the difference between a weaker binding affinity or a less stable protein. Of note, this was done with only the β receptor present.

The M5 mutant (N79S) was intended to be a thoroughly innocuous positive control. The N79S mutation removes a glycosylation site so E. coli is able to produce an identical protein as that produced in CHO, since cytokine glycosylation does not affect activity of the resultant protein. M5 appears to be roughly equipotent with the WT construct, supporting the design hypothesis. Glycosylation may be useful for increasing the molecular weight (MW) to reduce excretion, but additional research is needed in this area.

The M2 (N72R) mutant was reported as ˜1% WT activity, although predictions indicated that this mutant should be comparable to WT. The N72R mutation appears to be near equipotent with the WT construct. However, runs to test the mutant did not show a full curve, particularly at the top. The N72R mutant does appear to give a full response at the top dose (less active, as opposed to partial agonist). The reported mutant was a fusion construct, unlike here.

The other three mutants tested (N72Y, N79E) exhibited poor binding to a Ni column and poor recovery, although expression looked fine. The N72D mutant is the Altor Bioscience “superagonist” mutation (Zhu et al., J Immunol 183:3598-3607, 2009; Liu et al., J Biol Chem 291:23869-91, 2016).

### Example 25—IL-15 Binding to IL-154 with and without Receptor γ

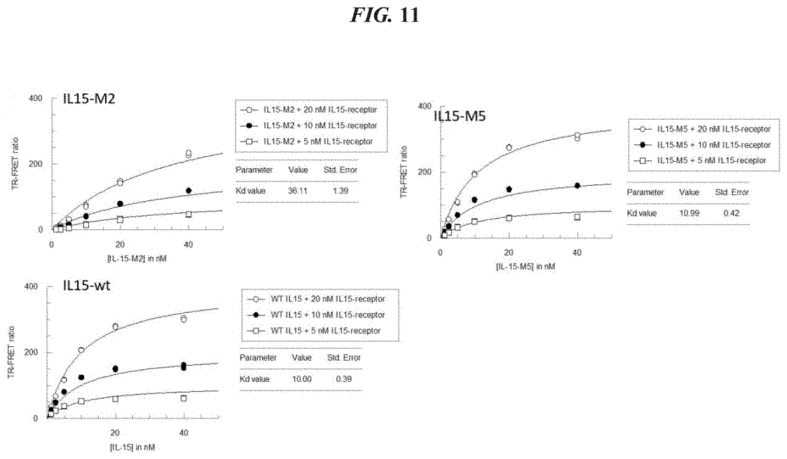

In order to see if there are effects on receptor γ, two sets of conditions were compared using in-house prepared rIL-15. The two conditions tested were (1) with (+) 20 nM receptor γ, and (2) without (-) receptor γ. As shown in FIG. 12, an 8-fold increase in apparent affinity was seen in the presence of the γ receptor. The measured K_{d} without the γ receptor was 28.5 nM, which was higher than previous analyses (10 nM). The increased affinity, in the presence of γ receptor was expected. The fits to the data are not as strong as desired, and further research is necessary to verify these results. The fits at the highest receptor concentration appear to be a less robust fit than the lower concentrations. The model used to fit the data assumes a more simplified binding model where γ and β act as one combined unit.

Table 6 provides a summary of IL-15 receptor binding efficiencies.

TABLE 6
IL-15 Receptor Binding Efficiencies

| | TR-FRET (Kd in nM) | | |
|---|---|---|---|
| Cytokine | | Exp 1 | Exp 2 |

| | | | | |
|---|---|---|---|---|
| IL-15 wt | 0.95 | nM | 0.88 | nM |
| IL-15 M2 | 17.26 | nM | 69.3 | nM |

| | | |
|---|---|---|
| IL-15 M5 | 1.24M | 1.87M |

### Example 26—IL-15-¹²⁵I Labeled Receptor-Ligand Binding Assay

IL-15 binding is determined by quantitating the ability of unlabeled recombinant IL-15 (wt or mutant) to compete with ¹²I-labeled IL-15 in binding to membrane extracts containing the IL-2Rβ receptor. Membrane extracts are obtained from the cell line M-07e (DSMZ #ACC 104) that have been grown to a density between 1.0×10⁶ and 1.5×10⁶ cells/ml. Approximately 7.5×10⁶ total cells are spun down for extraction using the Mem-PER™ Plus Protein Extraction Kit (Thermo Fisher Scientific #89842). Total protein concentration is determined using the Pierce™ BCA Protein Assay Kit (Thermo Fisher Scientific #23225). For each well to be tested, 1.5 μg of M-07e membrane extract is incubated at 4° C. with 0.25 mg of WGE PVT SPA Beads (Perkin Elmer #RPNQ0001) in a final volume of 20 μL×#experimental wells (excess volume is PBS pH 7.4+0.1% BSA) for 4 hours to overnight. After incubation, membrane bound beads are washed 2× with 1 ml of PBS/BSA and resuspended in a final volume of #experimental wells×50 μL of PBS/BSA. To 96 well white OptiPlates (Perkin Elmer #6005290), add 25 μL of unlabeled IL-15 in PBS/BSA at 4×designated concentration to designated wells on plate. Then add 25 μL of ¹²⁵I-labelled IL-15 (for IL-15—Sino Biological #10360-H07E; for ¹²⁵I-labelling—Perkin Elmer #CusReag1) at a 4×concentration of 3.6 nM (5 nCi/μl) to each well. To initiate binding 50 μL of beads bound to membrane are added to each well and then mixed by pipetting. Following a 2-hour incubation at RT the plates are spun at 750 rpm for 5 min. Binding of ¹²⁵I-labelled IL-15 is then measured with a TopCount scintillation counter (Hewlatt Packard). Binding affinity is determined by using the curve fitting graphing program GraFit (Erithacus Software) to determine the IC₅₀ values of the of the IL-15 recombinant protein variants using a 4 parameter IC₅₀ curve fitting equation.

$$CPM = \frac{Range}{1 + {( \frac{[ cold IL 15 ]}{{IC}_{50}} )}^{S}} + Background$$

The foregoing protocol and variants thereof may be used to assess competitive receptor binding of IL-15 and mutants thereof. It is expected that some IL-15 mutants will bind with equal or greater affinity to the receptor than the wild type.

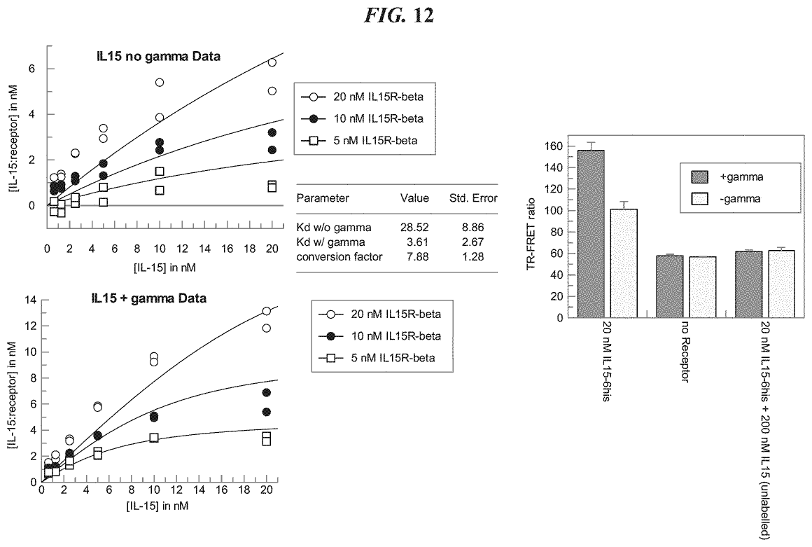

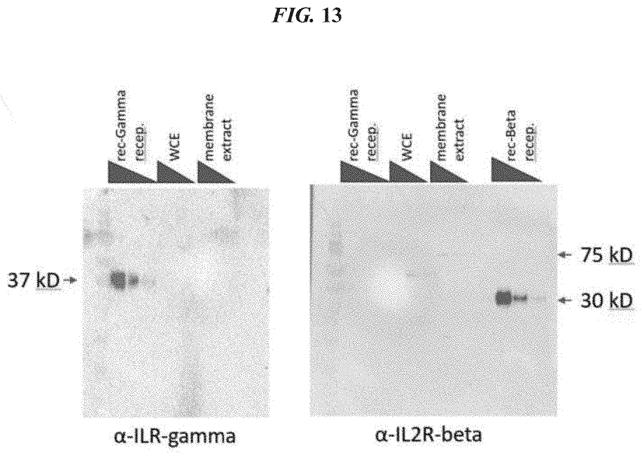

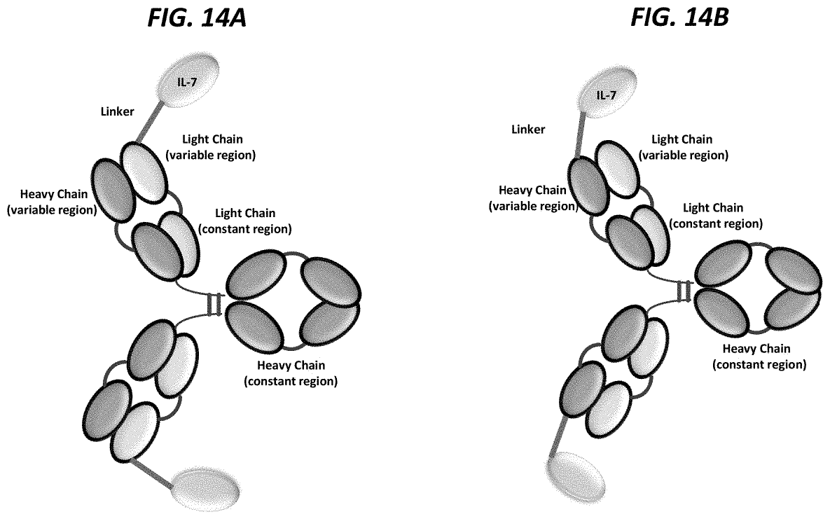

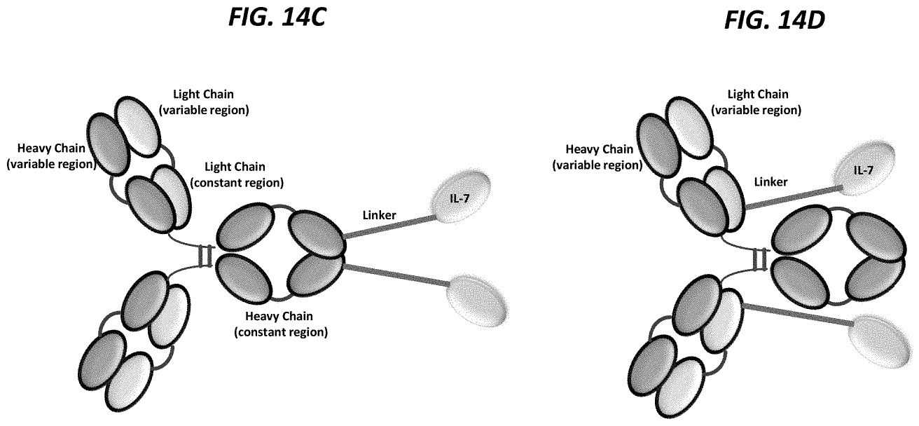

FIG. 13 shows western blots of M-07e membrane extracts. M-07e is a cell line that responds to IL-15. This cell line is a good source of receptors for ¹²⁵I-labelled binding of IL-15. Both the β and γ subunits of the receptor were tested, and the same blot was reprobed. As shown in the figure, recombinant bands of expected size seen on both images. The image of the α-γ antibody did not appear to show any bands, while the α-β probe showed faint bands at the expected MW. The lowest recombinant protein band represented 50 fmol of protein. Using ¹²⁵I, as low as 200 fmol/mg should be detectable. A faint band that is 10×less than what is in the rec. protein lane should still represent 1000 fmol/mg. Despite low concentration of receptor, they are likely to be enough for ¹²⁵I binding to detect in an assay.

### Example 27—Disclosed Amino Acid Sequences

TABLE 7
Amino Acid Sequences

| SEQ | | Mutation (if | |
|---|---|---|---|
| ID | Sequence | applicable, | |
| NO: | Description | underlined) | Sequence |
| 1 | Sequence of | | MFHVSFRYIFGLPPLILVLLPVASSDCDIEGKDGKQ |
| | human IL-7 | | YESVLMVSIDQLLDSMKEIGSNCLNNEFNFFKRHIC |
| | | | DANKEGMFLFRAARKLRQFLKMNSTGDFDLHLLK |
| | | | VSEGTTILLNCTGQVKGRKPAALGEAQPTKSLEEN |
| | | | KSLKEQKKLNDLCFLKRLLQEIKTCWNKILMGTKEH |
| 2 | Mature, | | DCDIEGKDGKQYESVLMVSIDQLLDSMKEIGSNCL |
| | truncated | | NNEFNFFKRHICDANKEGMFLFRAARKLRQFLKM |
| | sequence of | | NSTGDFDLHLLKVSEGTTILLNCTGQVKGRKPAAL |
| | human IL-7 | | GEAQPTKSLEENKSLKEQKKLNDLCFLKRLLQEIK |
| | (amino acids | | TCWNKILMGTKEH |
| | 26-177) | | |
| 3 | Sequence of | | MFHVSFRYIFGIPPLILVLLPVTSSDCHIKDKDGKAF |
| | rat IL-7 | | GSVLMISINQLDKMTGTDSDCPNNEPNFFKKHLCD |
| | | | DTKEAAFLNRAARKLRQFLKMNISEEFNDHLLRVS |
| | | | DGTQTLVNCTSKEEKTIKEQKKNDPCFLKRLLREI |
| | | | KTCWNKILKGSI |
| 4 | Sequence of | | MFHVSFRYIFGIPPLILVLLPVTSSECHIKDKEGKAY |
| | mouse IL-7 | | ESVLMISIDELDKMTGTDSNCPNNEPNFFRKHVCD |
| | | | DTKEAAFLNRAARKLKQFLKMNISEEFNVHLLTVS |
| | | | QGTQTLVNCTSKEEKNVKEQKKNDACFLKRLLREI |
| | | | KTCWNKILKGSI |
| 5 | Sequence of | | MFHVSFRYIFGLPPLILVLLPVASSDCDIEGKDGKQ |
| | monkey IL-7 | | YESVLMVSIDQLLDSMKEIGSNCLNNEFNFFKRHL |
| | | | CDDNKEGMFLFRAARKLKQFLKMNSTGDFDLHLL |
| | | | KVSEGTTILLNCTGKVKGRKPAALGEPQPTKSLEE |
| | | | NKSLKEQKKLNDSCFLKRLLQKIKTCWNKILMGT |
| | | | KEH |
| 6 | Sequence of | | MFHVSFRYIFGIPPLILVLLPVASSDCDISGKDGGAY |
| | cow IL-7 | | QNVLMVNIDDLDNMINFDSNCLNNEPNFFKKHSC |
| | | | DDNKEASFLNRASRKLRQFLKMNISDDFKLHLSTV |
| | | | SQGTLTLLNCTSKGKGRKPPSLSEAQPTKNLEENK |
| | | | SSKEQKKQNDLCFLKILLQKIKTCWNKILRGIKEH |
| 7 | Sequence of | | MFHVSFRYIFGIPPLILVLLPVASSDCDFSGKDGGA |
| | sheep IL-7 | | YQNVLMVSIDDLDNMINFDSNCLNNEPNFFKKHSC |
| | | | DDNKEASFLNRAARKLKQFLKMNISDDFKLHLST |
| | | | VSQGTLTLLNCTSKGKGRKPPSLGEAQPTKNLEEN |
| | | | KSLKEQRKQNDLCFLKILLQKIKTCWNKILRGITEH |
| 8 | IL-7 S1 | K10I | MDCDIEGKDGIQYESVLMVSIDQLLDSMKEIGSNC |
| | | | LNNEFNFFKRHICDANKEGMFLFRAARKLRQFLK |
| | | | MNSTGDFDLHLLKVSEGTTILLNCTGQVKGRKPA |
| | | | ALGEAQPTKSLEENKSLKEQKKLNDLCFLKRLLQE |
| | | | IKTCWNKILMGTKEH |
| 9 | IL-7 S2 | K10M | MDCDIEGKDGMQYESVLMVSIDQLLDSMKEIGSN |
| | | | CLNNEFNFFKRHICDANKEGMFLFRAARKLRQFLK |
| | | | MNSTGDFDLHLLKVSEGTTILLNCTGQVKGRKPA |
| | | | ALGEAQPTKSLEENKSLKEQKKLNDLCFLKRLLQE |
| | | | IKTCWNKILMGTKEH |
| 10 | IL-7 S3 | Q11R | MDCDIEGKDGKRYESVLMVSIDQLLDSMKEIGSNC |
| | | | LNNEFNFFKRHICDANKEGMFLFRAARKLRQFLK |
| | | | MNSTGDFDLHLLKVSEGTTILLNCTGQVKGRKPA |
| | | | ALGEAQPTKSLEENKSLKEQKKLNDLCFLKRLLQE |
| | | | IKTCWNKILMGTKEH |
| 11 | IL-7 S4 | S14T | MDCDIEGKDGKQYETVLMVSIDQLLDSMKEIGSN |
| | | | CLNNEFNFFKRHICDANKEGMFLFRAARKLRQFLK |
| | | | MNSTGDFDLHLLKVSEGTTILLNCTGQVKGRKPA |
| | | | ALGEAQPTKSLEENKSLKEQKKLNDLCFLKRLLQE |
| | | | IKTCWNKILMGTKEH |
| 12 | IL-7 S5 | S19Q | MDCDIEGKDGKQYESVLMVQIDQLLDSMKEIGSN |
| | | | CLNNEFNFFKRHICDANKEGMFLFRAARKLRQFLK |
| | | | MNSTGDFDLHLLKVSEGTTILLNCTGQVKGRKPA |
| | | | ALGEAQPTKSLEENKSLKEQKKLNDLCFLKRLLQE |
| | | | IKTCWNKILMGTKEH |
| 13 | IL-7 S6 | K10V | MDCDIEGKDGVQYESVLMVSIDQLLDSMKEIGSN |
| | | | CLNNEFNFFKRHICDANKEGMFLFRAARKLRQFLK |
| | | | MNSTGDFDLHLLKVSEGTTILLNCTGQVKGRKPA |
| | | | ALGEAQPTKSLEENKSLKEQKKLNDLCFLKRLLQE |
| | | | IKTCWNKILMGTKEH |
| 14 | IL-7 S7 | K81M | MDCDIEGKDGKQYESVLMVSIDQLLDSMKEIGSN |
| | | | CLNNEFNFFKRHICDANKEGMFLFRAARKLRQFLK |
| | | | MNSTGDFDLHLLMVSEGTTILLNCTGQVKGRKPA |
| | | | ALGEAQPTKSLEENKSLKEQKKLNDLCFLKRLLQE |
| | | | IKTCWNKILMGTKEH |
| 15 | IL-7 S8 | K81R | MDCDIEGKDGKQYESVLMVSIDQLLDSMKEIGSN |
| | | | CLNNEFNFFKRHICDANKEGMFLFRAARKLRQFLK |
| | | | MNSTGDFDLHLLRVSEGTTILLNCTGQVKGRKPAA |
| | | | LGEAQPTKSLEENKSLKEQKKLNDLCFLKRLLQEI |
| | | | KTCWNKILMGTKEH |
| 16 | IL-7 S9 | G85M | MDCDIEGKDGKQYESVLMVSIDQLLDSMKEIGSN |
| | | | CLNNEFNFFKRHICDANKEGMFLFRAARKLRQFLK |
| | | | MNSTGDFDLHLLKVSEMTTILLNCTGQVKGRKPA |
| | | | ALGEAQPTKSLEENKSLKEQKKLNDLCFLKRLLQE |
| | | | IKTCWNKILMGTKEH |
| 17 | IL-7 S1 with | K10I | MDCDIEGKDGIQYESVLMVSIDQLLDSMKEIGSNC |
| | a C-terminal | | LNNEFNFFKRHICDANKEGMFLFRAARKLRQFLK |
| | His-tag. | | MNSTGDFDLHLLKVSEGTTILLNCTGQVKGRKPA |
| | | | ALGEAQPTKSLEENKSLKEQKKLNDLCFLKRLLQE |
| | | | IKTCWNKILMGTKEHENLYFQGHHHHHH |
| 18 | IL-7 S2 with | K10M | MDCDIEGKDGMQYESVLMVSIDQLLDSMKEIGSN |
| | a C-terminal | | CLNNEFNFFKRHICDANKEGMFLFRAARKLRQFLK |
| | His-tag. | | MNSTGDFDLHLLKVSEGTTILLNCTGQVKGRKPA |
| | | | ALGEAQPTKSLEENKSLKEQKKLNDLCFLKRLLQE |
| | | | IKTCWNKILMGTKEHENLYFQGHHHHHH |
| 19 | IL-7 S3 with | Q11R | MDCDIEGKDGKRYESVLMVSIDQLLDSMKEIGSNC |
| | a C-terminal | | LNNEFNFFKRHICDANKEGMFLFRAARKLRQFLK |
| | His-tag. | | MNSTGDFDLHLLKVSEGTTILLNCTGQVKGRKPA |
| | | | ALGEAQPTKSLEENKSLKEQKKLNDLCFLKRLLQE |
| | | | IKTCWNKILMGTKEHENLYFQGHHHHHH |
| 20 | IL-7 S4 with | S14T | MDCDIEGKDGKQYETVLMVSIDQLLDSMKEIGSN |
| | a C-terminal | | CLNNEFNFFKRHICDANKEGMFLFRAARKLRQFLK |
| | His-tag. | | MNSTGDFDLHLLKVSEGTTILLNCTGQVKGRKPA |
| | | | ALGEAQPTKSLEENKSLKEQKKLNDLCFLKRLLQE |
| | | | IKTCWNKILMGTKEHENLYFQGHHHHHH |
| 21 | IL-7 S5 with | S19Q | MDCDIEGKDGKQYESVLMVQIDQLLDSMKEIGSN |
| | a C-terminal | | CLNNEFNFFKRHICDANKEGMFLFRAARKLRQFLK |
| | His-tag. | | MNSTGDFDLHLLKVSEGTTILLNCTGQVKGRKPA |
| | | | ALGEAQPTKSLEENKSLKEQKKLNDLCFLKRLLQE |
| | | | IKTCWNKILMGTKEHENLYFQGHHHHHH |
| 22 | IL-7 S6 with | K10V | MDCDIEGKDGVQYESVLMVSIDQLLDSMKEIGSN |
| | a C-terminal | | CLNNEFNFFKRHICDANKEGMFLFRAARKLRQFLK |
| | His-tag. | | MNSTGDFDLHLLKVSEGTTILLNCTGQVKGRKPA |
| | | | ALGEAQPTKSLEENKSLKEQKKLNDLCFLKRLLQE |
| | | | IKTCWNKILMGTKEHENLYFQGHHHHHH |
| 23 | IL-7 S7 with | K81M | MDCDIEGKDGKQYESVLMVSIDQLLDSMKEIGSN |
| | a C-terminal | | CLNNEFNFFKRHICDANKEGMFLFRAARKLRQFLK |
| | His-tag. | | MNSTGDFDLHLLMVSEGTTILLNCTGQVKGRKPA |
| | | | ALGEAQPTKSLEENKSLKEQKKLNDLCFLKRLLQE |
| | | | IKTCWNKILMGTKEHENLYFQGHHHHHH |
| 24 | IL-7 S8 with | K81R | MDCDIEGKDGKQYESVLMVSIDQLLDSMKEIGSN |
| | a C-terminal | | CLNNEFNFFKRHICDANKEGMFLFRAARKLRQFLK |
| | His-tag. | | MNSTGDFDLHLLRVSEGTTILLNCTGQVKGRKPAA |
| | | | LGEAQPTKSLEENKSLKEQKKLNDLCFLKRLLQEI |
| | | | KTCWNKILMGTKEHENLYFQGHHHHHH |
| 25 | IL-7 S9 with | G85M | MDCDIEGKDGKQYESVLMSIDQLLDSMKEIGSN |
| | a C-terminal | | CLNNEFNFFKRHICDANKEGMFLFRAARKLRQFLK |
| | His-tag. | | MNSTGDFDLHLLKVSEMTTILLNCTGQVKGRKPA |
| | | | ALGEAQPTKSLEENKSLKEQKKLNDLCFLKRLLQE |
| | | | IKTCWNKILMGTKEHENLYFQGHHHHHH |
| 26 | Cos-7 WT | | MFHVSFRYIFGLPPLILVLLPVASSDCDIEGKDGKQ |
| | Expressed | | YESVLMVSIDQLLDSMKEIGSNCLNNEFNFFKRHIC |
| | IL-7 with | | DANKEGMFLFRAARKLRQFLKMNSTGDFDLHLLK |
| | Leader | | VSEGTTILLNCTGQVKGRKPAALGEAQPTKSLEEN |
| | Sequence and | | KSLKEQKKLNDLCFLKRLLQEIKTCWNKILMGTKE |
| | His tag | | HENLYFQGHHHHHH |
| 27 | IL-7 WT | | MDCDIEGKDGKQYESVLMVSIDQLLDSMKE |
| | (commer- | | IGSNCLNNEFNFFKRHICDANKEGMFLFRA |
| | cially- | | ARKLRQFLKMNSTGDFDLHLLKVSEGTTIL |
| | sourced) | | LNCTGQVKGRKPAALGEAQPTKSLEENKSL |
| | | | KEQKKLNDLCFLKRLLQEIKTCWNKILMGTKEH |
| 28 | IL-7 WT (in- | | MDCDIEGKDGKQYESVLMVSIDQLLDSMKE |
| | house-His 6) | | IGSNCLNNEFNFFKRHICDANKEGMFLFRA |
| | | | ARKLRQFLKMNSTGDFDLHLLKVSEGTTIL |
| | | | LNCTGQVKGRKPAALGEAQPTKSLEENKSL |
| | | | KEQKKLNDLCFLKRLLQEIKTCWNKILMGTKEHE |
| | | | NLYFQGHHHHHH |
| 29 | Sequence of | | MRISKPHLRSISIQCYLCLLLNSHFLTEAGIHVFILG |
| | human IL-15 | | CFSAGLPKTEANWVNVISDLKKIEDLIQSMHIDATL |
| | | | YTESDVHPSCKVTAMKCFLLELQVISLESGDASIHD |
| | | | TVENLIILANNSLSSNGNVTESGCKECEELEEKNIK |
| | | | EFLQSFVHIVQMFINTS |
| 30 | Mature, | | NWVNVISDLKKIEDLIQSMHIDATLYTESDVHPSC |
| | truncated | | KVTAMKCFLLELQVISLESGDASIHDTVENLIILAN |
| | sequence of | | NSLSSNGNVTESGCKECEELEEKNIKEFLQSFVHIV |
| | human IL-15 | | QMFINTS |
| | (amino acids | | |
| | 49-162) | | |
| 31 | IL-15 M1 | N72D | NWVNVISDLKKIEDLIQSMHIDATLYTESDVHPSC |
| | | | KVTAMKCFLLELQVISLESGDASIHDTVENLIILAN |
| | | | DSLSSNGNVTESGCKECEELEEKNIKEFLQSFVHIV |
| | | | QMFINTS |
| 32 | IL-15 M2 | N72R | NWVNVISDLKKIEDLIQSMHIDATLYTESDVHPSC |
| | | | KVTAMKCFLLELQVISLESGDASIHDTVENLIILAN |
| | | | RSLSSNGNVTESGCKECEELEEKNIKEFLQSFVHIV |
| | | | QMFINTS |
| 33 | IL-15 M3 | N72Y | NWVNVISDLKKIEDLIQSMHIDATLYTESDVHPSC |
| | | | KVTAMKCFLLELQVISLESGDASIHDTVENLIILAN |
| | | | YSLSSNGNVTESGCKECEELEEKNIKEFLQSFVHIV |
| | | | QMFINTS |
| 34 | IL-15 M4 | N79E | NWVNVISDLKKIEDLIQSMHIDATLYTESDVHPSC |
| | | | KVTAMKCFLLELQVISLESGDASIHDTVENLIILAN |
| | | | NSLSSNGEVTESGCKECEELEEKNIKEFLQSFVHIV |
| | | | QMFINTS |
| 35 | IL-15 M5 | N79S | NWVNVISDLKKIEDLIQSMHIDATLYTESDVHPSC |
| | | | KVTAMKCFLLELQVISLESGDASIHDTVENLIILAN |
| | | | NSLSSNGSVTESGCKECEELEEKNIKEFLQSFVHIV |
| | | | QMFINTS |
| 36 | IL-15 M6 | K11L | NWVNVISDLKLIEDLIQSMHIDATLYTESDVHPSCK |
| | | | VTAMKCFLLELQVISLESGDASIHDTVENLIILANN |
| | | | SLSSNGNVTESGCKECEELEEKNIKEFLQSFVHIVQ |
| | | | MFINTS |
| 37 | IL-15 M7 | K11M | NWVNVISDLKMIEDLIQSMHIDATLYTESDVHPSC |
| | | | KVTAMKCFLLELQVISLESGDASIHDTVENLIILAN |
| | | | NSLSSNGNVTESGCKECEELEEKNIKEFLQSFVHIV |
| | | | QMFINTS |
| 38 | IL-15 M8 | N112H | NWVNVISDLKKIEDLIQSMHIDATLYTESDVHPSC |
| | | | KVTAMKCFLLELQVISLESGDASIHDTVENLIILAN |
| | | | NSLSSNGNVTESGCKECEELEEKNIKEFLQSFVHIV |
| | | | QMFIHTS |
| 39 | IL-15 M9 | N112M | NWVNVISDLKKIEDLIQSMHIDATLYTESDVHPSC |
| | | | KVTAMKCFLLELQVISLESGDASIHDTVENLIILAN |
| | | | NSLSSNGNVTESGCKECEELEEKNIKEFLQSFVHIV |
| | | | QMFIMTS |
| 40 | IL-15 M10 | V3I | NWINVISDLKKIEDLIQSMHIDATLYTESDVHPSCK |
| | | | VTAMKCFLLELQVISLESGDASIHDTVENLIILANN |
| | | | SLSSNGNVTESGCKECEELEEKNIKEFLQSFVHIVQ |
| | | | MFINTS |
| 41 | IL-15 M11 | V3M | NWMNVISDLKKIEDLIQSMHIDATLYTESDVHPSC |
| | | | KVTAMKCFLLELQVISLESGDASIHDTVENLIILAN |
| | | | NSLSSNGNVTESGCKECEELEEKNIKEFLQSFVHIV |
| | | | QMFINTS |
| 42 | IL-15 M12 | V3R | NWRNVISDLKKIEDLIQSMHIDATLYTESDVHPSCK |
| | | | VTAMKCFLLELQVISLESGDASIHDTVENLIILANN |
| | | | SLSSNGNVTESGCKECEELEEKNIKEFLQSFVHIVQ |
| | | | MFINTS |
| 43 | IL-15 M13 | N4H | NWVHVISDLKKIEDLIQSMHIDATLYTESDVHPSC |
| | | | KVTAMKCFLLELQVISLESGDASIHDTVENLIILAN |
| | | | NSLSSNGNVTESGCKECEELEEKNIKEFLQSFVHIV |
| | | | QMFINTS |
| 44 | IL-15 M14 | K11R | NWVNVISDLKRIEDLIQSMHIDATLYTESDVHPSCK |
| | | | VTAMKCFLLELQVISLESGDASIHDTVENLIILANN |
| | | | SLSSNGNVTESGCKECEELEEKNIKEFLQSFVHIVQ |
| | | | MFINTS |
| 45 | IL-15 M15 | N112Y | NWVNVISDLKKIEDLIQSMHIDATLYTESDVHPSC |
| | | | KVTAMKCFLLELQVISLESGDASIHDTVENLIILAN |
| | | | NSLSSNGNVTESGCKECEELEEKNIKEFLQSFVHIV |
| | | | QMFIYTS |
| 46 | IL-15 M1 | N72D | MGSSHHHHHHSSGENLYFQGHMNWVNVISDLKKI |
| | with an N- | | EDLIQSMHIDATLYTESDVHPSCKVTAMKCFLLEL |
| | terminal His | | QVISLESGDASIHDTVENLIILANDSLSSNGNVTESG |
| | tag. | | CKECEELEEKNIKEFLQSFVHIVQMFINTS |
| 47 | IL-15 M2 | N72R | MGSSHHHHHHSSGENLYFQGHMNWVNVISDLKKI |
| | with an N- | | EDLIQSMHIDATLYTESDVHPSCKVTAMKCFLELQ |
| | terminal His | | VISLESGDASIHDTVENLIILANRSLSSNGNVTESGC |
| | tag. | | KECEELEEKNIKEFLQSFVHIVQMFINTS |
| 48 | IL-15 M3 | N72Y | MGSSHHHHHHSSGENLYFQGHMNWVNVISDLKKI |
| | with an N- | | EDLIQSMHIDATLYTESDVHPSCKVTAMKCFLLEL |
| | terminal His | | QVISLESGDASIHDTVENLIILANYSLSSNGNVTESG |
| | tag. | | CKECEELEEKNIKEFLQSFVHIVQMFINTS |
| 49 | IL-15 M4 | N79E | MGSSHHHHHHSSGENLYFQGHMNWVNVISDLKKI |
| | with an N- | | EDLIQSMHIDATLYTESDVHPSCKVTAMKCFLLEL |
| | terminal His | | QVISLESGDASIHDTVENLIILANNSLSSNGEVTESG |
| | tag. | | CKECEELEEKNIKEFLQSFVHIVQMFINTS |
| 50 | IL-15 M5 | N79S | MGSSHHHHHHSSGENLYFQGHMNWVNVISDLKKI |
| | with an N- | | EDLIQSMHIDATLYTESDVHPSCKVTAMKCFLLEL |
| | terminal His | | QVISLESGDASIHDTVENLIILANNSLSSNGSVTESG |
| | tag. | | CKECEELEEKNIKEFLQSFVHIVQMFINTS |
| 51 | IL-15 M6 | K11L | MGSSHHHHHHSSGENLYFQGHMNWVNVISDLKLI |
| | with an N- | | EDLIQSMHIDATLYTESDVHPSCKVTAMKCFLLEL |
| | terminal His | | QVISLESGDASIHDTVENLIILANNSLSSNGNVTESG |
| | tag. | | CKECEELEEKNIKEFLQSFVHIVQMFINTS |
| 52 | IL-15 M7 | K11M | MGSSHHHHHHSSGENLYFQGHMNWVNVISDLKM |
| | with an N- | | IEDLIQSMHIDATLYTESDVHPSCKVTAMKCFLLEL |
| | terminal His | | QVISLESGDASIHDTVENLIILANNSLSSNGNVTESG |
| | tag. | | CKECEELEEKNIKEFLQSFVHIVQMFINTS |
| 53 | IL-15 M8 | N112H | MGSSHHHHHHSSGENLYFQGHMNWVNVISDLKKI |
| | with an N- | | EDLIQSMHIDATLYTESDVHPSCKVTAMKCFLLEL |
| | terminal His | | QVISLESGDASIHDTVENLIILANNSLSSNGNVTESG |
| | tag. | | CKECEELEEKNIKEFLQSFVHIVQMFIHTS |
| 54 | IL-15 M9 | N112M | MGSSHHHHHHSSGENLYFQGHMNWVNVISDLKKI |
| | with an N- | | EDLIQSMHIDATLYTESDVHPSCKVTAMKCFLLEL |
| | terminal His | | QVISLESGDASIHDTVENLIILANNSLSSNGNVTESG |
| | tag. | | CKECEELEEKNIKEFLQSFVHIVQMFIMTS |
| 55 | IL-15 M10 | V3I | MGSSHHHHHHSSGENLYFQGHMNWINVISDLKKI |
| | with an N- | | EDLIQSMHIDATLYTESDVHPSCKVTAMKCFLLEL |
| | terminal His | | QVISLESGDASIHDTVENLIILANNSLSSNGNVTESG |
| | tag. | | CKECEELEEKNIKEFLQSFVHIVQMFINTS |
| 56 | IL-15 M11 | V3M | MGSSHHHHHHSSGENLYFQGHMNWMNVISDLKK |
| | with an N- | | IEDLIQSMHIDATLYTESDVHPSCKVTAMKCFLLEL |
| | terminal His | | QVISLESGDASIHDTVENLIILANNSLSSNGNVTESG |
| | tag. | | CKECEELEEKNIKEFLQSFVHIVQMFINTS |
| 57 | IL-15 M12 | V3R | MGSSHHHHHHSSGENLYFQGHMNWRNVISDLKKI |
| | with an N- | | EDLIQSMHIDATLYTESDVHPSCKVTAMKCFLLEL |
| | terminal His | | QVISLESGDASIHDTVENLIILANNSLSSNGNVTESG |
| | tag. | | CKECEELEEKNIKEFLQSFVHIVQMFINTS |
| 58 | IL-15 M13 | N4H | MGSSHHHHHHSSGENLYFQGHMNWVHVISDLKKI |
| | with an N- | | EDLIQSMHIDATLYTESDVHPSCKVTAMKCFLLEL |
| | terminal His | | QVISLESGDASIHDTVENLIILANNSLSSNGNVTESG |
| | tag. | | CKECEELEEKNIKEFLQSFVHIVQMFINTS |
| 59 | IL-15 M14 | K11R | MGSSHHHHHHSSGENLYFQGHMNWVNVISDLKRI |
| | with an N- | | EDLIQSMHIDATLYTESDVHPSCKVTAMKCFLLEL |
| | terminal His | | QVISLESGDASIHDTVENLIILANNSLSSNGNVTESG |
| | tag. | | CKECEELEEKNIKEFLQSFVHIVQMFINTS |
| 60 | IL-15 M15 | N112Y | MGSSHHHHHHSSGENLYFQGHMNWVNVISDLKKI |
| | with an N- | | EDLIQSMHIDATLYTESDVHPSCKVTAMKCFLLEL |
| | terminal His | | QVISLESGDASIHDTVENLIILANNSLSSNGNVTESG |
| | tag. | | CKECEELEEKNIKEFLQSFVHIVQMFIYTS |
| 61 | IL-15 WT | | MNWVNVISDLKKIEDLIQSMHIDATLYTESDVHPS |
| | (commer- | | CKVTAMKCFLLELQVISLESGDASIHDTVENLIILA |
| | cially- | | NNSLSSNGNVTESGCKECEELEEKNIKEFLQSFVHI |
| | sourced) | | VQMFINTS |
| 62 | IL-15 WT | | MGSSHHHHHHSSGENLYFQGHMNWVNVISDLKKI |
| | (in-house | | EDLIQSMHIDATLYTESDVHPSCKVTAMKCFLLEL |
| | His6) | | QVISLESGDASIHDTVENLIILANNSLSSNGNVTESG |
| | | | CKECEELEEKNIKEFLQSFVHIVQMFINTS |

### Example 28—Anti-CD47 Fusion Proteins

Fusion polypeptides disclosed herein comprise a human heavy chain variable domain and a light chain variable domain combined with a human kappa or any human Fc IgG constant domain, respectively. A glycine-serine linker (G4S)n at either the C or N terminus of the light or heavy antibody chain was designed to link IL-7 (wildtype or mutant) to the antibody polypeptide. These fusion constructs will be designed to incorporate a secretion signal and cloned into a mammalian expression system, and transfected into CHO cells to generate antibody fusion proteins. The protein variants were expressed, secreted into the medium, and purified using protein A resin.

Some of the anti-CD47 fusion proteins comprises a first, heavy polypeptide chain and a second, light polypeptide chain, which first polypeptide chain comprises: a first domain comprising a binding region of a heavy chain variable domain of an immunoglobulin (V_{H}), a constant region of the heavy chain (C_{H}), and (ii) a modified/mutant IL-7 or a modified/mutant IL-15 sequence fused to the C-terminus of the constant region of the heavy chain (C_{H}); and a second polypeptide chain which comprises: binding region of a light chain variable domain of an immunoglobulin (V_{L}) and (ii) a constant region of the light chain (C_{L}).

Some of the anti-CD47 fusion proteins comprises a first, heavy polypeptide chain and a second, light polypeptide chain, which first polypeptide chain comprises: a first domain comprising a binding region of a heavy chain variable domain of an immunoglobulin (V_{H}) and a constant region of the heavy chain (C_{H}), and a second polypeptide which comprises: a binding region of a light chain variable domain of an immunoglobulin (V_{L}), a constant region of the light chain (C_{L}), and a modified/mutant IL-7 or a modified/mutant IL-15 sequence fused to the C-terminus of the constant region of the light chain (C_{L}).

Some of the anti-CD47 fusion proteins comprises a first, heavy polypeptide chain and a second, light polypeptide chain, which first polypeptide chain comprises: a first domain comprising a binding region of a heavy chain variable domain of an immunoglobulin (V_{H}) and a constant region of the heavy chain (C_{H}), and a second polypeptide which comprises: a modified/mutant IL-7 or a modified/mutant IL-15 sequence fused to the N-terminus of the binding region of a light chain variable domain and a constant region of the light chain (C_{L}).

Some of the anti-CD47 fusion proteins comprises a first, heavy polypeptide chain and a second, light polypeptide chain, which first polypeptide chain comprises: a modified/mutant IL-7 or a modified/mutant IL-15 sequence fused to the N-terminus of the binding region of a heavy chain variable domain of an immunoglobulin (V_{H}) and constant region of the heavy chain (C_{H}), and a second polypeptide which comprises: a binding region of a light chain variable domain of an immunoglobulin (V_{L}) and a constant region of the light chain (C_{L}).

In one embodiment the modified/mutant IL-7 sequence and the V_{H} or C_{H} domains of anti-CD47 fusion proteins may be separated by a short linker with a repeated sequence of GGGGS, wherein the short linker can be represented as (GGGGS)n, with n having a value of 0, 1, 2, 3, 4, 5, 6, 7, 8, 9, 10, 11, 12, 13, 14, 15, 16, 17, 18, 19, 20. The linker needs to be of sufficient length to minimize steric hindrance between each of the binding domains. In some embodiments the linker is glycine and/or serine.

In another embodiment, the modified/mutant IL-15 sequence and the V_{H} or C_{H} domains of anti-CD47 fusion proteins may be separated by a short linker with a repeated sequence of GGGGS, wherein the short linker can be represented as (GGGGS)n, with n having a value of 0, 1, 2, 3, 4, 5, 6, 7, 8, 9, 10, 11, 12, 13, 14, 15, 16, 17, 18, 19, 20. The linker needs to be of sufficient length to minimize steric hindrance between each of the binding domains. In some embodiments the linker is glycine and/or serine.

Anti-CD47 fusion proteins comprising a first, heavy chain fusion polypeptide and a second, light chain fusion polypeptide can be constructed with the modified/mutant IL-7 or modified/mutant IL-15 sequences disclosed in Table 7 using methods described herein and known in the art which are not limiting.

### Example 29-Anti-CD47-IL-7 and Anti-CD47-IL-15 Fusion Proteins Bind to Recombinant Human CD47

The binding of anti-CD47-IL-7 fusion proteins, anti-CD47-IL-15 fusion proteins and anti-CD47 mAbs to human CD47 expressed on cells will be measured in vitro. For the in vitro binding to recombinant CD47, His-CD47 (AcroBiosystems) will be absorbed to high-binding microtiter plates overnight at 4° C. The wells will be washed, blocked, and increasing concentrations of anti-CD47-IL-7 fusion proteins, anti-CD47-IL-15 fusion proteins, anti-CD47 antibody (control), or control IgG antibody (control) will be added to the wells for 1 hour. The wells will be washed and then incubated with HRP-labelled secondary antibody for 1 hour, then washed, and developed with the addition of peroxidase substrate.

It is expected that the anti-CD47-IL-7 fusion proteins will bind to His-CD47 in a concentration-dependent manner and with similar affinity as the as an anti-CD47 antibody (control). It is expected, the negative control IgG antibodies will not bind to the His-CD47.

It is expected that an anti-CD47-IL-15 fusion protein constructed with a modified/mutant IL-15 as disclosed in Table 7, will bind to His-CD47 in a concentration-dependent manner with similar affinity to an anti-CD47 antibody and to anti-CD47-IL-7 fusion proteins described in Example 30.

### Example 30—Anti-CD47-IL-7 and Anti-CD47-IL-15 Fusion Proteins Activate STAT5 Phosphorylation in T Cells

To assess the effect of anti-CD47-IL-7 and anti-CD47-IL-15 fusion proteins on pSTAT5 activity in PBMC derived T cells in vitro, the following method using flow cytometry will be employed.

Human-PBMCs will be obtained by leukapheresis of human peripheral blood and incubated in tissue culture grade flasks in RPMI (Corning, Catalog #10-104-CV) with 10% fetal bovine serum (BioWest; Catalog #S01520) and penicillin/streptomycin (Corning, Catalog #30-001-CI) overnight at 37° C. For in vitro pSTAT5 stimulation assays, 1-3×105 PBMC per 200 RPMI media will be plated per well in a 96-well tissue culture treated plate. An 11-fold serial dilution series of fusion proteins (0.04-30 μg/mL) will be added to the PBMCs cultures and incubated at 37° C. for 15 minutes. The cells will be washed once with ice-cold PBS then fixed with Fix Buffer 1 (BD Phosflow, Catalog #557870). PBMC were washed twice with staining buffer (PBS+1% FBS) and permeabilized with Perm Buffer III (BD Phosflow, Catalog #558050). Cells will be washed twice with staining buffer, stained with Brilliant-blue 515 conjugated anti-human CD3 antibodies (BD Biosciences, Catalog #564465) and anti-pSTAT5-PE (BD Biosciences, Catalog #562077) and analyzed by flow cytometry using an Attune NxT flow cytometer (Life Technologies) for the percentage of CD3-positive T cells that are positive for pSTAT5.

It is expected that anti-CD47-IL-7 and anti-CD47-IL-15 fusion proteins will increase phosphorylation of STAT5 in T cells in a concentration-dependent manner.

It is expected that an anti-CD47-IL-7 fusion protein constructed with a modified/mutant IL-7, anti-CD47-IL-15 fusion protein constructed with a modified/mutant IL-15 as disclosed in Table 7, a combination of an anti-CD47 mAb with IL-7, IL-15, or a modified/mutant variant thereof, will increase phosphorylation of STAT5 in T cells in a concentration-dependent manner.

### Example 31—Transactivation of pSTAT5 in Human T Cells by Anti-CD47-IL-7 and Anti-CD47-IL-15 Fusion Proteins

To assess the ability of anti-CD47-IL-7 and CD47-IL-15 fusion proteins on pSTAT5 activity in PBMC derived T cells in vitro after binding to tumor cells, the following method using flow cytometry will be employed.

OV10-315 ovarian tumor cells that overexpress human CD47 will be seeded in 96 well plates overnight. Human-PBMCs will be obtained by leukapheresis of human peripheral blood and incubated in tissue culture grade flasks in RPMI (Corning, Catalog #10-104-CV) with 10% fetal bovine serum (BioWest; Catalog #S01520) and penicillin/streptomycin (Corning, Catalog #30-001-CI) overnight at 37° C. Increasing concentrations of anti-CD47-IL-7 or anti-CD47-IL15 fusion proteins will be incubated with the OV10-315 cells for 1 hour. Following six washes with PBS containing 1% FBS to remove all unbound protein, 1-3×10⁵ PBMC per 200 RPMI media will be plated per well and incubated at 37° C. for 15 minutes. The cells will be washed once with ice-cold PBS then fixed with Fix Buffer 1 (BD Phosflow, Catalog #557870). PBMC will be washed twice with staining buffer (PBS+1% FBS) and permeabilized with Perm Buffer III (BD Phosflow, Catalog #558050). Cells will be washed twice with staining buffer, stained with Brilliant-blue 515 conjugated anti-human CD3 antibodies (BD Biosciences, Catalog #564465) and anti-pSTAT5-PE (BD Biosciences, Catalog #562077) and analyzed by flow cytometry using an Attune NxT flow cytometer (Life Technologies) for the percentage of CD3-positive T cells that are positive for pSTAT5.

It is expected that fusion proteins will increase phosphorylation of STAT5 in T cells after binding to CD47 expression tumor cells in a concentration-dependent manner.

It is expected that anti-CD47-IL-7 and anti-CD47-IL-15 fusion proteins constructed with a modified/mutant IL-7 or a modified/mutant IL-15, respectively as disclosed in Table 7, and a combination of an anti-CD47 mAb with IL-7, IL-15, or a modified/mutant variant thereof, will increase phosphorylation of STAT5 in T cells after binding to tumor cells in a concentration-dependent manner.

### Example 32—Anti-CD47-IL-7 and Anti-CD47-IL-15 Fusion Proteins Aid in Survival of T Cells

To assess the effect of anti-CD47-IL-7 and anti-CD47-IL-15 fusion proteins on PBMC derived T cell survival in vitro, the following method using flow cytometry will be employed.

Human-PBMCs will be obtained by leukapheresis of human peripheral blood and incubated in tissue culture grade flasks in RPMI (Corning, Catalog #10-104-CV) with 10% fetal bovine serum (BioWest; Catalog #S01520) and penicillin/streptomycin (Corning, Catalog #30-001-CI) overnight at 37° C. For in vitro proliferation assays, 1×10⁵PBMC per 200 μL RPMI media will be plated per well in a 96-well tissue culture treated plate. An 11-fold serial dilution series of anti-CD47-IL-7 or anti-CD47-IL-15 fusion proteins (0.04-30 pg/mL) will be added to the PBMCs cultures and incubated at 37° C. for 4 days. The cells will be incubated with 5-ethynyl-2-deoxyuridine (EdU, Invitrogen, Catalog #C10634) for 24 hours prior to culture completion. EdU will be detected using an Alexafluor 647 picolyl azide reagent, stained with Brilliant-blue 515 conjugated anti-hCD3 antibodies (BD Biosciences, Catalog #564465) and analyzed by flow cytometry using an Attune NxT flow cytometer (Life Technologies).

It is expected that the anti-CD47-IL-7 and anti-CD47-IL-15 fusion proteins will contribute to fluorescent signal accumulation in the nuclei of cells where DNA has been synthesized during the EdU incubation period indicating an increase in survival and proliferation.

It is expected that anti-CD47-IL-7 and anti-CD47-IL-15 fusion proteins constructed with a modified/mutant IL-7 or a modified/mutant IL-15, respectively, as disclosed in Table 7, and a combination of an anti-CD47 mAb with IL-7, IL-15, or a modified/mutant variant thereof, will contribute to fluorescent signal accumulation in the nuclei of cells where DNA has been synthesized during the EdU incubation period indicating an increase in survival and proliferation.

### Example 33—Transactivation by Anti-CD47-IL-7 and Anti-CD47-IL-15 Fusion Proteins Aid in Survival of T Cells

To assess the ability of anti-CD47-IL-7 and anti-CD47-IL-15 fusion proteins on PBMC derived T cell survival in vitro after binding to tumor cells, the following method using flow cytometry will be employed.

Irradiated or chemically treated OV10-315 ovarian tumor cells that overexpress human CD47 will be seeded in 96 well plates overnight. Human-PBMCs will be obtained by leukapheresis of human peripheral blood and incubated in tissue culture grade flasks in RPMI (Corning, Catalog #10-104-CV) with 10% fetal bovine serum (BioWest; Catalog #S01520) and penicillin/streptomycin (Corning, Catalog #30-001-CI) overnight at 37° C. Increasing concentration of Anti-CD47-IL-7 or anti-CD47-IL-15 fusion proteins will be incubated with the OV10-315 cells for 1 hour. Following six washes with PBS containing 1% FBS to remove all unbound protein, 1×105 PBMC per 200 μL RPMI media will be plated per well and incubated at 37° C. for 4 days. The cells will be incubated with EdU (Invitrogen, Catalog #C10634) for 24 hours prior to culture completion. EdUu is detected using an Alexafluor 647 picolyl azide reagent, stained with Brilliant-blue 515 conjugated anti-human CD3 antibodies (BD Biosciences, Catalog #564465) and analyzed by flow cytometry using an Attune NxT flow cytometer (Life Technologies).

It is expected that the anti-CD47-IL-7 and anti-CD47-IL-15 fusion proteins will contribute to fluorescent signal accumulation in the nuclei of cells where DNA has been synthesized during the EdU incubation period indicating an increase in survival and proliferation.

It is expected that anti-CD47-IL-7 and anti-CD47-IL-15 fusion proteins constructed with a modified/mutant IL-7 or a modified/mutant IL-15, respectively, as disclosed in Table 7, and a combination of an anti-CD47 mAb with IL-7, IL-15, or a modified/mutant variant thereof, will contribute to fluorescent signal accumulation in the nuclei of cells where DNA has been synthesized during the EdU incubation period indicating an increase in survival and proliferation.

### Example 34—Anti-Tumor Activity of Anti-CD47-IL-7 and Anti-CD47-IL-15 Fusion Proteins in an OV90 Ovarian Xenograft Model in a Humanized NSG Mice

The anti-tumor properties of anti-CD47-IL-7 and anti-CD47_IL-15 fusion proteins and combinations of an anti-CD47 mAbs with IL-7, IL-15, or a modified/mutant variant thereof in an OV90 ovarian xenograft model in a humanized NSG mice will be evaluated.

NSG mice are humanized by adoptive transfer using human umbilical cord blood-derived CD34+ stem cells from a qualified source, following myeloablation treatment. CD34+ stem cells develop into human immune cells that engraft within the immunodeficient NSG mice.

Models engrafted with cord blood-derived hematopoietic stem cells (HSC) develop multi-lineage engraftment and display robust T-cell maturation and T-cell dependent inflammatory responses.

Humanized female NSG mice (NOD-Cg-PrkdcscidI12rgtm1Wjl/SzJ, Jackson Laboratories) will be implanted subcutaneously in the right flank with 0.1 mL of a 30% RPMI/70% Matrigel™ (BD Biosciences; Bedford, Mass.) mixture containing a suspension of 5×10⁶ OV90 ovarian carcinoma cells (ATCC). Digital calipers will be used to measure width and length diameters of the tumor. Tumor volumes will be calculated utilizing the formula: tumor volume (mm³)=(a×b²/2); where ‘b’ is the smallest diameter and ‘a’ is the largest diameter. Mice with palpable tumor volumes of 50-100 mm³ will be randomized into 10 mice/group. Mice will be treated with 1) human IgG control (10 mg/kg); 2) selected anti-CD47-IL-7 Fusion proteins (10 mg/kg); 3) selected anti-CD47-IL-15 fusion proteins (10 mg/kg); 4) anti-CD47 mAb; 5)anti-CD47 mAb+IL-7; 6) anti-CD47 mAb+IL-15; 7) anti-CD47 mAb+IL-7 modified/mutant variant; and 8) anti-CD47 mAb+IL-15 modified/mutant variant by an appropriate regimen, i.e., five days per week for a total of 6 weeks (QD5×6) by intraperitoneal injection (IP) or once per week for a total of 6 weeks by intravenously injection (IV).

Mean tumor growth inhibition (TGI) will be calculated utilizing the following formula. Mice exhibiting tumor shrinkage (TS) will be excluded from the TGI calculations.

$$T G I = [ 1 - \frac{( \overline{X} {Treated}_{( final )} - \overline{X} {Treated}_{( Day 0 )} )}{( \overline{X} Vehicle {Control}_{( final )} - \overline{X} Vehicle {Control}_{( Day 0 )} )} ] \times 100 \%$$

Significant differences in tumor volume will be confirmed using a two-way ANOVA, unpaired, parametric with the Tukey's Multiple Comparison test.

It is expected, except for the control group, that all treated groups: 1) human IgG control (10 mg/kg); 2) selected anti-CD47-IL-7 Fusion proteins (10 mg/kg); 3) selected anti-CD47-IL-15 fusion proteins (10 mg/kg); 4) anti-CD47 mAb; 5) anti-CD47 mAb+IL-7; 6) anti-CD47 mAb+IL-15; 7) anti-CD47 mAb+IL-7 modified/mutant variant; and 8) anti-CD47 mAb+IL-15 modified/mutant variant will result in statistically significant inhibition of tumor growth at 10 mg/kg daily dosing in OV90 human ovarian xenograft model.

### Example 35—Anti-Tumor Activity of Anti-CD47-IL-7 and Anti-CD47-IL-15 Fusion Proteins in a SNU-1 Gastric Carcinoma Xenograft Model in Humanized NSG Mice

The anti-tumor properties of anti-CD47-IL-7 fusion proteins and combinations of an anti-CD47 mAb with IL-7, IL-15, or a modified/mutant variant thereof in a SNU-1 gastric carcinoma xenograft model in NSG mice will be evaluated.

NSG mice are humanized by adoptive transfer using human umbilical cord blood-derived CD34+ stem cells from a qualified source, following myeloablation treatment. CD34+ stem cells develop into human immune cells that engraft within the immunodeficient NSG mice.

Models engrafted with cord blood-derived hematopoietic stem cells (HSC) develop multi-lineage engraftment and display robust T-cell maturation and T-cell dependent inflammatory responses.

Humanized female NSG mice (NOD-Cg-PrkdcscidI12rgtm1Wjl/SzJ, Jackson Laboratories) will be inoculated subcutaneously in the right flank with 0.1 mL of a 30% RPMI/70% Matrigel™ (BD Biosciences; Bedford, Mass.) mixture containing a suspension of 5×106 SNU-1 gastric carcinoma cells (ATCC). Eight days following inoculation, digital calipers will be used to measure width and length diameters of the tumor. Tumor volumes will be calculated utilizing the formula: tumor volume (mm3)=(a×b2/2) where ‘b’ is the smallest diameter and ‘a’ is the largest diameter. Mice with palpable tumor volumes of 50-100 mm3 will be randomized into 10 mice/group. Mice will be treated with 1) human IgG control (10 mg/kg); 2) selected anti-CD47-IL-7 Fusion proteins (10 mg/kg); 3) selected anti-CD47-IL-15 fusion proteins (10 mg/kg); 4) anti-CD47 mAb; 5) anti-CD47 mAb+IL-7; 6) anti-CD47 mAb+IL-15; 7) anti-CD47 mAb+IL-7 modified/mutant variant; and 8) anti-CD47 mAb+IL-15 modified/mutant variant by an appropriate regimen, i.e., five days per week for a total of 6 weeks (QD5×6) by intraperitoneal injection (IP) or once per week for a total of 6 weeks by intravenously injection (IV).

Mean tumor growth inhibition (TGI) will be calculated utilizing the following formula. Mice exhibiting tumor shrinkage (TS) will be excluded from the TGI calculations.

$$T G I = [ 1 - \frac{( \overline{X} {Treated}_{( final )} - \overline{X} {Treated}_{( Day 0 )} )}{( \overline{X} Vehicle {Control}_{( final )} - \overline{X} Vehicle {Control}_{( Day 0 )} )} ] \times 100 \%$$

Significant differences in tumor volume will be confirmed using a two-way ANOVA, unpaired, parametric with the Tukey's Multiple Comparison test.

It is expected, except for the control group, that all treated groups: 1) human IgG control (10 mg/kg) 2) selected anti-CD47-IL-7 Fusion proteins (10 mg/kg); 3) selected anti-CD47-IL-15 fusion proteins (10 mg/kg); 4) anti-CD47 mAb; 5)anti-CD47 mAb+IL-7; 6) anti-CD47 mAb+IL-15; 7) anti-CD47 mAb+IL-7 modified/mutant variant; and 8) anti-CD47 mAb+IL-15 modified/mutant variant will result in SNU-1 tumor growth inhibition, demonstrating anti-tumor efficacy in vivo.

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

1. A polypeptide comprising a first and a second polypeptide chain, wherein:
a. the first polypeptide chain comprises (i) a first domain comprising a binding region of a light chain variable domain of an immunoglobulin (V_{L}) specific for human CD47; and (ii) a second domain comprising a light chain constant domain (C_{L}); and the second polypeptide chain comprises (i) a first domain comprising a binding region of a heavy chain variable domain of an immunoglobulin (V_{H}) specific for human CD47; (ii) a second domain comprising a heavy chain constant domain (C_{H}); and (iii) a third domain comprising an IL-7 protein, IL-15 protein, or variant thereof;
b. the first polypeptide chain comprises (i) a first domain comprising a binding region of a light chain variable domain of an immunoglobulin (V_{L}) specific for human CD47; (ii) a second domain comprising a light chain constant domain (C_{L}); and (iii) a third domain comprising an IL-7 protein, IL-15 protein, or variant thereof; and the second polypeptide chain comprises (i) a first domain comprising a binding region of a heavy chain variable domain of an immunoglobulin (V_{H}) specific for human CD47; and (ii) a second domain comprising a heavy chain constant domain (C_{H});
c. the first polypeptide chain comprises (i) a first domain comprising an IL-7 protein, IL-15 protein, or variant thereof; (ii) a second domain comprising a binding region of a light chain variable domain of an immunoglobulin (V_{L}) specific for human CD47; and (iii) a third domain comprising a light chain constant domain (C_{L}); and the second polypeptide chain comprises (i) a first domain comprising a binding region of a heavy chain variable domain of an immunoglobulin (V_{H}) specific for human CD47, and (ii) a second domain comprising a heavy chain constant domain (C_{H}); or
d. the first polypeptide chain comprises (i) a first domain comprising a binding region of a light chain variable domain of an immunoglobulin (V_{L}) specific for human CD47, and (ii) a second domain comprising a light chain constant domain (C_{L}); and the second polypeptide chain comprises (i) a first domain comprising an IL-7 protein, IL-15 protein, or variant thereof; (ii) a second domain comprising a binding region of a heavy chain variable domain of an immunoglobulin (V_{H}) specific for human CD47; and (iii) a third domain comprising a heavy chain constant domain (C_{H}).

2.-4. (canceled)

5. The polypeptide of claim 1, wherein the IL-7 protein or variant thereof
(i) is modified;
(ii) is capable of binding to an IL-7 receptor to activate IL-7 signaling in a cell; or
(iii) comprises a substitution of an amino acid.

6.-7. (canceled)

8. The polypeptide of claim 5, wherein the amino acid substitution in the IL-7 protein or variant thereof is in the binding region for the IL-7 receptor;
wherein the amino acid substitution is a substitution in an amino acid position chosen from amino acid positions 10, 11, 14, 19, 81, and 85;
wherein the amino acid position is relative to SEQ ID NO:2; and
wherein the amino acid substitution
(i) at amino acid position 10 is K10I, K10M, or K10V;
(ii) at amino acid position 11 is Q11R;
(iii) at amino acid position 14 is S14T;
(iv) at amino acid position 19 is S19Q;
(v) at amino acid position 81 is K81M or K81R; or
(vi) at amino acid position 85 is G85M.

9.-15. (canceled)

16. A method of treating cancer in a subject, comprising administering to the subject the polypeptide of claim 1, wherein the cancer is a solid tumor or a hematologic malignancy.

17.-25. (canceled)

26. The method of claim 16, wherein the subject is further administered an anti-cancer agent, wherein the anti-cancer agent is a proteasome inhibitor or an immune checkpoint inhibitor.

27.-32. (canceled)

33. A pharmaceutical composition for use in treating a cancer in a subject in need thereof comprising the polypeptide of claim 1.

34. A method for treating a cancer in a subject in need thereof comprising administering to the subject a therapeutically effective amount of:
a) an anti-CD47 antibody or antigen binding fragment thereof; and
b) an IL-7 protein;
wherein the cancer is a solid tumor or a hematologic malignancy.

35. The method of claim 34, wherein the anti-CD47 antibody or antigen binding fragment thereof comprises a combination of a heavy chain (HC) and a light chain (LC), wherein the combination is chosen from:
(i) a heavy chain comprising the amino acid sequence of SEQ ID NO:64 and a light chain comprising the amino acid sequence SEQ ID NO:68;
(ii) a heavy chain comprising the amino acid sequence of SEQ ID NO:65 and a light chain comprising the amino acid sequence SEQ ID NO:68;
(iii) a heavy chain comprising the amino acid sequence of SEQ ID NO:63 and a light chain comprising the amino acid sequence SEQ ID NO:67;
(iv) a heavy chain comprising the amino acid sequence of SEQ ID NO:64 and a light chain comprising the amino acid sequence SEQ ID NO:67;
(v) a heavy chain comprising the amino acid sequence of SEQ ID NO:65 and a light chain comprising the amino acid sequence SEQ ID NO:67; and
(vi) a heavy chain comprising the amino acid sequence of SEQ ID NO:66 and a light chain comprising the amino acid sequence SEQ ID NO:67.

36. The method of claim 34,
wherein the IL-7 protein has an amino acid sequence at least about 70%, at least about 80%, at least about 85%, at least about 90%, at least about 95%, at least about 96%, at least about 97%, at least about 98%, at least about 99%, or about 100% identical to the amino acid sequence of SEQ ID NO. 1 (GenBank Accession No. P13232);
wherein the IL-7 protein is a fusion protein; and wherein the fusion protein comprises a heterologous moiety; or
wherein the IL-7 protein is modified; and wherein
(i) the modified IL-7 protein is capable of binding to an IL-7 receptor to activate IL-7 signaling in a cell; or
(ii) the modified IL-7 protein comprises a substitution of an amino acid;
wherein the amino acid substitution in the modified IL-7 protein is a substitution in an amino acid position selected from the group consisting of amino acid positions 10, 11, 14, 19, 81, and 85; wherein the amino acid positions are relative to SEQ ID NO:2; wherein the amino acid substitution
a. at amino acid position 10 is K10I, K10M, or K10V;
b. at amino acid position 11 is Q11R;
c. at amino acid position 14 is S14T;
d. at amino acid position 19 is 519Q;
e. at amino acid position 81 is K81M or K81R; or
f. at amino acid position 85 is G85M.

37.-67. (canceled)

68. The method of claim 34, wherein the subject is further administered an anti-cancer agent-, wherein the anti-cancer agent is a proteasome inhibitor or an immune checkpoint inhibitor.

69.-73. (canceled)

74. A modified IL-7 protein comprising at least one amino acid substitution as shown in SEQ ID NOs:8-16; wherein
(i) the modified IL-7 protein is capable of binding to an IL-7 receptor to activate IL-7 signaling in a cell; or
(ii) the amino acid substitution in the modified IL-7 protein is a substitution in an amino acid position selected from the group consisting of amino acid positions 10, 11, 14, 19, 81, and 85; wherein the amino acid positions are relative to SEQ ID NO:2; and wherein the amino acid substitution
a. at amino acid position 10 is K10I, K10M, or K10V;
b. at amino acid position 11 is Q11R;
c. at amino acid position 14 is S14T;
d. at amino acid position 19 is S19Q;
e. at amino acid position 81 is K81M or K81R; or
f. at amino acid position 85 is G85M.

75.-83. (canceled)

84. A nucleic acid construct encoding the modified IL-7 protein of any of claim 75; wherein the modified IL-7 protein further comprises a C-terminal Histidine tag.

85. (canceled)

86. A method of improving expansion and persistence of a chimeric antigen receptor (CAR)-bearing immune effector cell, comprising administering the CAR-bearing immune effector cell to a patient along with the modified IL-7 protein of claim 77;
wherein the modified IL-7 protein is capable of binding to an IL-7 receptor to activate IL-7 signaling in the CAR-bearing immune effector cell;
wherein the CAR-bearing immune effector cell is selected from a CAR-T cell, a CAR-iNKT cell, or a CAR-NK cell; or
wherein the modified IL-7 protein and the CAR-bearing immune effector cell are administered concurrently with a drug.

87. A method of initiating internal signaling in a CAR-bearing immune effector cell, comprising:
administering the modified IL-7 protein of claim 77 to a patient in need thereof,
wherein the modified IL-7 protein binds an IL-7 receptor; and
wherein binding of the modified IL-7 protein initiates internal signaling in the CAR-bearing immune effector cell;
wherein the CAR-bearing immune effector cell is selected from a CAR-T cell, a CAR-iNKT cell, or a CAR-NK cell; or
wherein the modified IL-7 protein and the CAR-bearing immune effector cell are administered concurrently with a drug.

88. A method of treating cancer in a subject in need thereof, comprising administering to the subject the modified IL-7 protein of claim 77 and a CAR-bearing immune effector cell;
wherein the modified IL-7 protein is capable of binding to an IL-7 receptor to activate IL-7 signaling in the CAR-bearing immune effector cell;
wherein the CAR-bearing immune effector cell is selected from a CAR-T cell, a CAR-iNKT cell, or a CAR-NK cell; or
wherein the modified IL-7 protein and the CAR-bearing immune effector cell are administered concurrently with a drug.

89.-91. (canceled)

92. A modified IL-15 protein comprising at least one amino acid substitution as shown in SEQ ID NOs:31-45; wherein
(i) the modified IL-15 protein is capable of binding to an IL-15 receptor to activate IL-15 signaling in a cell; or
(ii) the amino acid substitution in the modified IL-15 protein is a substitution in an amino acid position selected from the group consisting of amino acid positions 3, 4, 11, 72, 79, and 112; wherein the amino acid positions are relative to SEQ ID NO:30; and wherein the amino acid substitution
a. at amino acid position 3 is V3I, V3M, or V3R;
b. at amino acid position 4 is N4H;
c. at amino acid position 11 is K11L, K11M, or K11R;
d. at amino acid position 72 is N72D, N72R or N72Y;
e. at amino acid position 79 is N79E or N79S; or
f. at amino acid position 112 is N112H, N112M, or N112Y.

93.-101. (canceled)

102. A nucleic acid construct encoding the modified IL-15 protein of claim 93; wherein the modified IL-15 protein further comprises an N-terminal Histidine tag.

103. (canceled)

104. A method of improving expansion and persistence of an immune effector cell, e.g., a chimeric antigen receptor (CAR)-bearing immune effector cell, comprising administering the CAR-bearing immune effector cell to a patient along with the modified IL-15 protein of claim 95;
wherein the modified IL-15 protein is capable of binding to an IL-15 receptor to activate IL-15 signaling in the CAR-bearing immune effector cell;
wherein the CAR-bearing immune effector cell is selected from a CAR-T cell, a CAR-iNKT cell, or a CAR-NK cell; or
wherein the modified IL-15 protein and the CAR-bearing immune effector cell are administered concurrently with a drug.

105. A method of initiating internal signaling in an immune effector cell, e.g., a CAR-bearing immune effector cell, comprising:
administering the modified IL-15 protein of claim 95 to a patient in need thereof,
wherein the modified IL-15 protein binds an IL-15 receptor; and
wherein binding of the modified IL-15 protein initiates internal signaling in the CAR-bearing immune effector cell;
wherein the CAR-bearing immune effector cell is selected from a CAR-T cell, a CAR-iNKT cell, or a CAR-NK cell; or
wherein the modified IL-15 protein and the CAR-bearing immune effector cell are administered concurrently with a drug.

106. A method of treating cancer in a subject in need thereof, comprising administering to the subject the modified IL-15 protein of claim 95 and a CAR-bearing immune effector cell;
wherein the modified IL-15 protein is capable of binding to an IL-15 receptor to activate IL-15 signaling in the CAR-bearing immune effector cell;
wherein the CAR-bearing immune effector cell is selected from a CAR-T cell, a CAR-iNKT cell, or a CAR-NK cell; or
wherein the modified IL-15 protein and the CAR-bearing immune effector cell are administered concurrently with a drug.

107.-112. (canceled)

113. The polypeptide of claim 1, wherein the immunoglobulin variable region specific for human CD47 is connected to an IL-7 protein, IL-7 variants, IL-15 protein, or IL-15 variant by a linker-, wherein said linker is (GGGGS)n, wherein n =0, 1, 2, 3, 4, 5, 6, 7, 8, 9, 10, 11, 12, 13, 14, 15, 16, 17, 18; wherein said immunoglobulin variable region is from an anti-CD47 monoclonal antibody or fragment thereof.

114.-115. (canceled)


SEQUENCE LISTING (68 sequences)

SEQ ID NO: 1 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 3 - protein, 154 aa, Rattus rattus
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 4 - protein, 154 aa, Mus musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 5 - protein, 177 aa, Pan troglodytes
MFHVSFRYIFGLPPLILVLLPVASSDCDIEGKDGKQYESVLMVSIDQLLDSMKEIGSNCLNNEFNFFKRHLCDDNKEGMFLFRAARKLKQFLKMNSTGDFDLHLLKVSEGTTILLNCTGKVKGRKPAALGEPQPTKSLEENKSLKEQKKLNDSCFLKRLLQKIKTCWNKILMGTKEH

SEQ ID NO: 6 - protein, 176 aa, Bos taurus
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 7 - protein, 176 aa, Ovis aries
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 8 - protein, 153 aa, Artificial sequence
  synthetic construct
MDCDIEGKDGIQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 9 - protein, 153 aa, Artificial Sequence
  synthetic construct
MDCDIEGKDGMQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 10 - protein, 153 aa, Artificial Sequence
  synthetic construct
MDCDIEGKDGKRYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 11 - protein, 153 aa, Artificial Sequence
  synthetic construct
MDCDIEGKDGKQYETVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 12 - protein, 153 aa, Artificial Sequence
  synthetic construct
MDCDIEGKDGKQYESVLMVQIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 13 - protein, 153 aa, Artificial Sequence
  synthetic construct
MDCDIEGKDGVQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 14 - protein, 153 aa, Artificial Sequence
  synthetic construct
MDCDIEGKDGKQYESVLMVSIDQLLDSMKEIGSNCLNNEFNFFKRHICDANKEGMFLFRAARKLRQFLKMNSTGDFDLHLLMVSEGTTILLNCTGQVKGRKPAALGEAQPTKSLEENKSLKEQKKLNDLCFLKRLLQEIKTCWNKILMGTKEH

SEQ ID NO: 15 - protein, 153 aa, Artificial Sequence
  synthetic construct
MDCDIEGKDGKQYESVLMVSIDQLLDSMKEIGSNCLNNEFNFFKRHICDANKEGMFLFRAARKLRQFLKMNSTGDFDLHLLRVSEGTTILLNCTGQVKGRKPAALGEAQPTKSLEENKSLKEQKKLNDLCFLKRLLQEIKTCWNKILMGTKEH

SEQ ID NO: 16 - protein, 153 aa, Artificial Sequence
  synthetic construct
MDCDIEGKDGKQYESVLMVSIDQLLDSMKEIGSNCLNNEFNFFKRHICDANKEGMFLFRAARKLRQFLKMNSTGDFDLHLLKVSEMTTILLNCTGQVKGRKPAALGEAQPTKSLEENKSLKEQKKLNDLCFLKRLLQEIKTCWNKILMGTKEH

SEQ ID NO: 17 - protein, 166 aa, Artificial Sequence
  synthetic construct
MDCDIEGKDGIQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHENLYFQGHHHHHH

SEQ ID NO: 18 - protein, 166 aa, Artificial Sequence
  synthetic construct
MDCDIEGKDGMQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHENLYFQGHHHHHH

SEQ ID NO: 19 - protein, 166 aa, Artificial Sequence
  synthetic construct
MDCDIEGKDGKRYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHENLYFQGHHHHHH

SEQ ID NO: 20 - protein, 166 aa, Artificial Sequence
  synthetic construct
MDCDIEGKDGKQYETVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHENLYFQGHHHHHH

SEQ ID NO: 21 - protein, 166 aa, Artificial Sequence
  synthetic construct
MDCDIEGKDGKQYESVLMVQIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHENLYFQGHHHHHH

SEQ ID NO: 22 - protein, 166 aa, Artificial Sequence
  synthetic construct
MDCDIEGKDGVQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHENLYFQGHHHHHH

SEQ ID NO: 23 - protein, 166 aa, Artificial Sequence
  synthetic construct
MDCDIEGKDGKQYESVLMVSIDQLLDSMKEIGSNCLNNEFNFFKRHICDANKEGMFLFRAARKLRQFLKMNSTGDFDLHLLMVSEGTTILLNCTGQVKGRKPAALGEAQPTKSLEENKSLKEQKKLNDLCFLKRLLQEIKTCWNKILMGTKEHENLYFQGHHHHHH

SEQ ID NO: 24 - protein, 166 aa, Artificial Sequence
  synthetic construct
MDCDIEGKDGKQYESVLMVSIDQLLDSMKEIGSNCLNNEFNFFKRHICDANKEGMFLFRAARKLRQFLKMNSTGDFDLHLLRVSEGTTILLNCTGQVKGRKPAALGEAQPTKSLEENKSLKEQKKLNDLCFLKRLLQEIKTCWNKILMGTKEHENLYFQGHHHHHH

SEQ ID NO: 25 - protein, 166 aa, Artificial Sequence
  synthetic construct
MDCDIEGKDGKQYESVLMVSIDQLLDSMKEIGSNCLNNEFNFFKRHICDANKEGMFLFRAARKLRQFLKMNSTGDFDLHLLKVSEMTTILLNCTGQVKGRKPAALGEAQPTKSLEENKSLKEQKKLNDLCFLKRLLQEIKTCWNKILMGTKEHENLYFQGHHHHHH

SEQ ID NO: 26 - protein, 190 aa, Artificial Sequence
  synthetic construct
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHENLYFQGHHHHHH

SEQ ID NO: 27 - protein, 153 aa, Artificial Sequence
  synthetic construct
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 28 - protein, 166 aa, Artificial Sequence
  synthetic construct
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHENLYFQGHHHHHH

SEQ ID NO: 29 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 30 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 31 - protein, 114 aa, Artificial Sequence
  synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 32 - protein, 114 aa, Artificial Sequence
  synthetic construct
NWVNVISDLKKIEDLIQSMHIDATLYTESDVHPSCKVTAMKCFLLELQVISLESGDASIHDTVENLIILANRSLSSNGNVTESGCKECEELEEKNIKEFLQSFVHIVQMFINTS

SEQ ID NO: 33 - protein, 114 aa, Artificial Sequence
  synthetic construct
NWVNVISDLKKIEDLIQSMHIDATLYTESDVHPSCKVTAMKCFLLELQVISLESGDASIHDTVENLIILANYSLSSNGNVTESGCKECEELEEKNIKEFLQSFVHIVQMFINTS

SEQ ID NO: 34 - protein, 114 aa, Artificial Sequence
  synthetic construct
NWVNVISDLKKIEDLIQSMHIDATLYTESDVHPSCKVTAMKCFLLELQVISLESGDASIHDTVENLIILANNSLSSNGEVTESGCKECEELEEKNIKEFLQSFVHIVQMFINTS

SEQ ID NO: 35 - protein, 114 aa, Artificial Sequence
  synthetic construct
NWVNVISDLKKIEDLIQSMHIDATLYTESDVHPSCKVTAMKCFLLELQVISLESGDASIHDTVENLIILANNSLSSNGSVTESGCKECEELEEKNIKEFLQSFVHIVQMFINTS

SEQ ID NO: 36 - protein, 114 aa, Artificial Sequence
  synthetic construct
NWVNVISDLKLIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 37 - protein, 114 aa, Artificial Sequence
  synthetic construct
NWVNVISDLKMIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 38 - protein, 114 aa, Artificial Sequence
  synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIHTS

SEQ ID NO: 39 - protein, 114 aa, Artificial Sequence
  synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIMTS

SEQ ID NO: 40 - protein, 114 aa, Artificial Sequence
  synthetic construct
NWINVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 41 - protein, 114 aa, Artificial Sequence
  synthetic construct
NWMNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 42 - protein, 114 aa, Artificial Sequence
  synthetic construct
NWRNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 43 - protein, 114 aa, Artificial Sequence
  synthetic construct
NWVHVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 44 - protein, 114 aa, Artificial Sequence
  synthetic construct
NWVNVISDLKRIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 45 - protein, 114 aa, Artificial Sequence
  synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIYTS

SEQ ID NO: 46 - protein, 136 aa, Artificial Sequence
  synthetic construct
MGSSHHHHHHSSGENLYFQGHMNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 47 - protein, 135 aa, Artificial Sequence
  synthetic construct
MGSSHHHHHHSSGENLYFQGHMNWVNVISDLKKIEDLIQSMHIDATLYTESDVHPSCKVTAMKCFLELQVISLESGDASIHDTVENLIILANRSLSSNGNVTESGCKECEELEEKNIKEFLQSFVHIVQMFINTS

SEQ ID NO: 48 - protein, 136 aa, Artificial Sequence
  synthetic construct
MGSSHHHHHHSSGENLYFQGHMNWVNVISDLKKIEDLIQSMHIDATLYTESDVHPSCKVTAMKCFLLELQVISLESGDASIHDTVENLIILANYSLSSNGNVTESGCKECEELEEKNIKEFLQSFVHIVQMFINTS

SEQ ID NO: 49 - protein, 136 aa, Artificial Sequence
  synthetic construct
MGSSHHHHHHSSGENLYFQGHMNWVNVISDLKKIEDLIQSMHIDATLYTESDVHPSCKVTAMKCFLLELQVISLESGDASIHDTVENLIILANNSLSSNGEVTESGCKECEELEEKNIKEFLQSFVHIVQMFINTS

SEQ ID NO: 50 - protein, 136 aa, Artificial Sequence
  synthetic construct
MGSSHHHHHHSSGENLYFQGHMNWVNVISDLKKIEDLIQSMHIDATLYTESDVHPSCKVTAMKCFLLELQVISLESGDASIHDTVENLIILANNSLSSNGSVTESGCKECEELEEKNIKEFLQSFVHIVQMFINTS

SEQ ID NO: 51 - protein, 136 aa, Artificial Sequence
  synthetic construct
MGSSHHHHHHSSGENLYFQGHMNWVNVISDLKLIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 52 - protein, 136 aa, Artificial Sequence
  synthetic construct
MGSSHHHHHHSSGENLYFQGHMNWVNVISDLKMIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 53 - protein, 136 aa, Artificial Sequence
  synthetic construct
MGSSHHHHHHSSGENLYFQGHMNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIHTS

SEQ ID NO: 54 - protein, 136 aa, Artificial Sequence
  synthetic construct
MGSSHHHHHHSSGENLYFQGHMNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIMTS

SEQ ID NO: 55 - protein, 136 aa, Artificial Sequence
  synthetic construct
MGSSHHHHHHSSGENLYFQGHMNWINVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 56 - protein, 136 aa, Artificial Sequence
  synthetic construct
MGSSHHHHHHSSGENLYFQGHMNWMNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 57 - protein, 136 aa, Artificial Sequence
  synthetic construct
MGSSHHHHHHSSGENLYFQGHMNWRNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 58 - protein, 136 aa, Artificial Sequence
  synthetic construct
MGSSHHHHHHSSGENLYFQGHMNWVHVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 59 - protein, 136 aa, Artificial Sequence
  synthetic construct
MGSSHHHHHHSSGENLYFQGHMNWVNVISDLKRIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 60 - protein, 136 aa, Artificial Sequence
  synthetic construct
MGSSHHHHHHSSGENLYFQGHMNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIYTS

SEQ ID NO: 61 - protein, 115 aa, Artificial Sequence
  synthetic construct
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 62 - protein, 136 aa, Artificial Sequence
  synthetic construct
MGSSHHHHHHSSGENLYFQGHMNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 63 - protein, 443 aa, Artificial Sequence
  synthetic construct
QVQLVQSGAEVKKPGASVKVSCKASGYTFTNYWIHWVRQAPGQGLEWMGYTDPRTDYTEYNQKFKDRVTMTRDTSTSTVYMELSSLRSEDTAVYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 64 - protein, 443 aa, Artificial Sequence
  synthetic construct
EVQLVQSGAEVKKPGESLKISCKGSGYTFTNYWIHWVRQMPGKGLEWMGYTDPRTDYTEYNQKFKDQVTISADKSISTAYLQWSSLKASDTAMYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 65 - protein, 443 aa, Artificial Sequence
  synthetic construct
QVQLVQSGAEVKKPGSSVKVSCKASGYTFTNYWIHWVRQAPGQGLEWMGYTDPRTDYTEYAQKFQGRVTITADESTSTAYMELSSLRSEDTAVYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 66 - protein, 443 aa, Artificial Sequence
  synthetic construct
EVQLVQSGAEVKKPGESLKISCKGSGYTFTNYWIHWVRQMPGKGLEWMGYTDPRTDYTEYSPSFQGQVTISADKSISTAYLQWSSLKASDTAMYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - protein, 219 aa, Artificial Sequence
  synthetic construct
DIVMTQSPDSLAVSLGERATINCRSSQNIVQSNGNTYLEWYQQKPGQPPKLLIYKVFHRFSGVPDRFSGSGSGTDFTLTISSLQAEDVAVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 219 aa, Artificial Sequence
  synthetic construct
DVVMTQSPLSLPVTLGQPASISCRSSQNIVQSNGNTYLEWFQQRPGQSPRRLIYKVFHRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC